SEQUENCE LISTING (35 sequences)

SEQ ID NO: 1 - protein, 159 aa, Acyrthosiphon pisum
MLNLKVMMFLCLSVIVVYCESDQVPINSSAAVESCLLETNMTRDEFEDMLTSPNARELTILKSHAHKCMFGCVMRKNHIVNDGVVSKEVLSKYVLNFYGRPDYKRRLIIKDVEHIVDVCAKKVADESETDECELAATLVTCIVLEANKAGLVDDPARQI

SEQ ID NO: 2 - protein, 243 aa, Acyrthosiphon pisum
MKVSAATAVLVALVATVQSSDPCNISTCYKSGTTKPPMAVTPTHLPVQSSSTQTSHPQTTYAKDHVHGSTTTKSGVNATVTTASGASVNGTEPPAVVKSSAGVTGNSTTPKPTMTEGHVALKQKLNTIAVKCKDELHAPQEIMALVSNTVVPQNEQQRCYLECVYKNLNLIKNNKFSVEDGKAMARIRFANQPEEHKKAVTIIETCEKEAVIDPKTTEKCAAGRVIRNCFVKNGEKINFFPKA

SEQ ID NO: 3 - protein, 141 aa, Acyrthosiphon pisum
MISSTFYITLVFGIAMLISCGHGRFTTEQIDYYGKACNASEDDLVVVKSYKVPTTETGKCLMKCMITKLGLLNDDGSYNKTGMEAGLKKYWSEWSTEKIESINNKCYEEALLVSKEVVATCNYSYTVMACLNKQLDLDKST

SEQ ID NO: 4 - protein, 193 aa, Acyrthosiphon pisum
MRGNYSSMVFLLFAIGFQDIFCQKQEPSGKCRAPDKAPLNLEIIINTCQEEIKSALLQEALDILNDGNVEQNTPNYSSRSKREAEEDLTNEERRVAGCLLQCVYKKVKAVDETGFPVVDGLMKLYNEGVQDRNYYIATLSAVRHCISIAQQLKQQQPSKSFDDGQTCDLAYEMFECVSEKIEENCGVENKSNN

SEQ ID NO: 5 - protein, 221 aa, Acyrthosiphon pisum
MSANSATIKCIAVAAILLQISVIFADAGHHRRGKELLDTEDSDFFRCKQASRKSCCGPENAMKRFGDKDKVAADECYAQVAEKFATVTATTPKQDLFSAEAVKITKKKQFCLHECIGKKNNLLTEDGSLNKTFIADYAMKSVFKEQWQKQVGQKALDKCLEETYIPWPAEDKENVCNPVYVQFQHCLWLQYESNCPANKIKITKKCEKTRNRYRMQKSTSN

SEQ ID NO: 6 - protein, 215 aa, Acyrthosiphon pisum
MQKVVFICIFAIICQTVFTAGYDRTWILRQKRGTNDDECRTLLPSSEKKLPSCCQMPNILPNLDSTWEKCFETFKQFKDKPETKEYKEMAHGKEPPCLFQCIFMQSGLTTSDGKLNEDAITKKMSEGINNDEKWKSIWQNSLNKCFDDVKQEDKKQILIMNTPAGRLMKCFLRDMYMSCPKNVWVESSECLNMKDLVQKCPEMPPPVFKSPPKLI

SEQ ID NO: 7 - protein, 155 aa, Acyrthosiphon pisum
MVARKRMYNMLPTTVLFAIIAATVLKDCDAYLSEAAIKKTQQMLKTVCSKKHSVEEDVFTNIKKGIFPEDNNNIKCYFACNFKTMQLINQKGVIDKKMFKDKMSMMAPPNVYKILLPVIEQCTGKDKGEELCQSSYNVIKCAHSVDPKSLEFLPL

SEQ ID NO: 8 - protein, 162 aa, Acyrthosiphon pisum
MFALKVACLCLSVAVVFGENNQQNGPSDRSATIFQSCIAETKLSGDALKGFRSMSIPKTQAEKCMMGCLMRKVNVINKGKFSVEEATKVAQKYYGTNEAMMKKAKDLIDVCAKKAQSTTEECALAGIVTTCIVEEAQKAGLSGGPGSRSRRTVSPKFRRDAM

SEQ ID NO: 9 - protein, 165 aa, Acyrthosiphon pisum
MIIKKTLLLSVFVLFGCLFSINKADDADAKDKELMSKLFTVVFKCFKDADWGTCGEMITTKYDITQAKYKQCTCHMACAGEELGMINASGQPEPAKFLEYVNKINNPDIKSQLQLIYDKCQNVKGSEKCDLAEQFAICAFKESPALKERVSTLMEMLVKMKPKSK

SEQ ID NO: 10 - protein, 143 aa, Acyrthosiphon pisum
MEHLRSTNVVFAIVMALLVVQSSTRPQPDEMEEIKRTLYNACAGKFPITEEIKNNAKNSIISDDPTFKCFLKCCFDEMSMIDEDGIIDGDSLKAMAPDHIKPILEQVIPSCTKNVKQDGCEASFEFISCGIKLNPLIVALLPL

SEQ ID NO: 11 - protein, 169 aa, Acyrthosiphon pisum
MSSSTFYITLLFGIAMLISCGYGIFTTEQIDYYGKACNASEDDLIVLKSYKVPSTETGKCLMKCMITKLGLLNDDGSYNKTGMEAGLKKYWSEWATEKIETINEKCYEEGNTATLLYHVAIYFTCVSGDYSDVQLLIHCDGMFEQEVGSRQVNLKLLIMLKIGLSEPKR

SEQ ID NO: 12 - protein, 463 aa, Acyrthosiphon pisum [sequence omitted]

SEQ ID NO: 13 - protein, 403 aa, Acyrthosiphon pisum
MDVMQKPERFILTPFQKFCIRWSVFFDSSSDRLSRIETVLRTIQFSTIMITSGMTMTSVLIADNKKALESFTYFVICVFMLAIITFAIRTKRFNRAMLLMVVDEFPGYNRPMPDVLKRKMAAIRTSYGDFTMKVIVSYLTLVLFEIPATAMVPLAAASLTDVKLGSQSTQMVVLWFPADTSQVGMYAVSYVIQFLIVVTVKFIITGIMCSFSFFVSQMISEFQILSAYVEHAVEIVEYDQSADKTTEQKLLDHVKNCVMLHDRLIYFKDQLNESYGYIILLELMFSTLYFCLSAFNMIFVGNRFVMVKGLLTLSNYLAELFIFCMYGSMVEDAHMGLLRASYSVAWYAQPVRFRQSLTMVMSRTQTPLQLTVGKVFIANLPLFLSVLKVSYSGVNALRAANAK

SEQ ID NO: 14 - protein, 386 aa, Acyrthosiphon pisum
MTSIGKKNQRKFYQTLMTLAFFLDTSQYRYISRFVKQFYIFDWMVLVSVAAAFTILEGNYRMPFVMELIQYMIVGFYFTSIFVVFIIKKEAIMSNYNCIQTKFIQWSNKRALHSNAAYKRNIKTVKSLSIPLAILSLSIALGPLISTINDIGKLPLDNRAHFVLFWPTIVDTNKLSMYGIIYTLQVIFTIILYISVLSFNLGYMVFLNELITQFEMLLNGINDAFKYKMDKQFQTLFIDCIRHHQIIIKFLDDLKSYFKWMILIEIIVVQVILAILIYNLTKVNASLGYKVKIAGSILFNLLPICFHCHVGEVVLSLHTRLSNHIYNMPWYDMPNKNKQLIVIMLQRTQRDLTLSSALFSSERASRSLISKVIKQVYTILNVLLKT

SEQ ID NO: 15 - protein, 367 aa, Acyrthosiphon pisum
MQRIDTINMFLQMTGCTDSKAMLYLTYFEFLITFYYLIATYASIVHFEQSVTIQLFALLCMLIECVILLNITFRLYHKNHIREMHQYSRRLGIPDSYRSVINVITKYHLIASNIFVVFPVTYAIFCDSVRVGDPFTFPFLDVLPMHTDNLAIYACKYLVYAISVYIAHVELCFINTTFIYYVGVLKHRLETIVQTIGEAFADNDEQKFKYAIIQHQKLLSYFNTMKIVFSKPILLSMSFNAIYFGLTTSFVIQAIRGYINQAILSICIASSAAAVINITIYTFYGSELMDLHDKILHVLFDNAFFYVSKSFKSSILIMMTRVTIPLKFTVGYIFTINLNLLLKILKMSYTVLNVLLSSETIKPHKLS

SEQ ID NO: 16 - protein, 369 aa, Acyrthosiphon pisum
MAHIVDIFFQNMGCSHDHGYGMVFFNCCELAITLFFTVSTYPTIADPTQNLSIRLYGVLCLLIEAHIFAFIAVRIYHQSQHRDMYQHLHGVEIPENYRRKIATVIKHHFIISNVFVAVSVLYTISLDWVRIGDPFTFPFIDVLPIKTTNVTVYVCKYIVYALPVYFAHLETCFLNVTFMFSVGIVKRHFQILNDQVEEAIVNEDEQKLKIAIKHHQQVLKYFEDMKTVYEKPILMTIEFCGLYVGLTSCFVIQVIQGFIHQIILGLCIVSSIACLMTIIIYCIYASNMYALHNGILNALFEHRSCYSRNKSFKRIILIMMTRATIPLEIKAGSVFTINLNLLVKILKFAYTVFNVLLSSINRQFKETAI

SEQ ID NO: 17 - protein, 430 aa, Acyrthosiphon pisum
MTTTPRVTELTAPASEDLTIVDNRLFKAICLHQILDPTKGGNRYYRLAFMVVMWVSLSVQIIQLVGLYFAVNDLQRFAFTTTVIFNALLCLSKGYVLVVNADRLRASLEVARYEFTSCGARNQRLVRRSRAVLSTILRTFAVLSWVTCFIWALTPLFAMDEYLQVTNADGTVSRYRVTIYNVWLPVPATVYNETTVWSLVYAVEVIACFVNVFSWLLFDSYVVTMCFTFNAQFRTVSASTTIGHHSDSFRSPPPHAPEGTSDDNNTFNCYDELINRIKDNQSIIKIYDDFFEILQPAILFQIIGGSYSVITLIFLTSLTYLMGFSIISIPVLKVFFGFLSVTFELFLYCYVFNHIETEKCNMNFGLYSSNWTAMDLKFKKTLLFAMNTNSSHRRVMKVTPMSIINLEMFANVMNMSYSIVSVLLNSRVQK

SEQ ID NO: 18 - protein, 420 aa, Acyrthosiphon pisum
MRSSSATVVDVMLFKAIGLYQLLCPADRGGYSVRSRRALMTALGLSFALHSFQVPYLYYALNDLQRFAYMAAVIIYGMMCSFKGYVLVTNADRLWLVLNAADYGYTGCGHRDPSRLRRCRATLSALLRTFVALSYGTLIVWIVLPFFVDEYTGITNSDGTVTRYRTTIHNMQYPIPLAVYNSRPVWALIYVTELYVCIVNVFIWSLFDCYLVTMCFVLNAQFHTMSAGYGTLGIRRTGSSPPDTTFAGVRRIKFDEIESNHYSDLISHIQDNQNLIKMFDVFFEVVRPVVLVQIANGSYSVISLIFLTALMYLMGVPVLSAAFLKFICGLISLTIELFIFCYGFNHIETAKSVLNFGIYSSNWTEMDLTFKKTMLLTMKMNSSHKRAMKVSPNSAVGLEMFARVMNMSYSTVSVLLNSRS

SEQ ID NO: 19 - protein, 388 aa, Acyrthosiphon pisum
FKHQGLVADLLPNIRVMQGVGHFMFNYYSEGKKFPHRIYCIVTLLLLLLQYGMMAVNLMMESDDVDDLTANTITMLFFLHPIVKMIYFPVRSKIFYKTLAIWNNPNSHPLFAESNARFHALAITKMRRLLFCVAGATIFSVISWTGITFIEDSPIPRLMIRTFYPFNAMSGAGHVFALIYQFYYLVISMAVSNSLDVLFCSWLLFACEQLQHLKAIMKPLMELSATGLTKKQEMLVRSAIKYWVERHKHVVRLVTAVGDAYGVALLLHMLTTTITLTLLAYQATKVNGVNVYAATVIGYLLYTLGQVFLFCIFGNRLIEESSSVMEAAYSCHWYDGSEEAKTFVQIVCQQCQKAMSISGAKFFTVSLDLFASVLGAVVTYFMVLVQLK

SEQ ID NO: 20 - protein, 414 aa, Acyrthosiphon pisum
MNLNDEQNYIVNLKLMKITGFYHLISSRAPKYFGFNVYKVTAAIEVMTGIFSIIMLFLSSYYYLDNTNELMSHFMLVVAIFFSTLKIFWVSRNSETIWNNMDMTCINFLSYTGHKKEILKKARAKSISTTILFVILWSSVTVAWSISPFFVKDVYLNIKFKDETRRFRYNSLNYVYPISEEFYNEHFLYFYVVEMLSVVFWGHGTVAYDTFVISICITIAFQLKTIAVSYISLNDKKGDIKNLKDNDLEAMFNLKLLIQDQQNMFKKIKEIYKIFEPVTFVQLAAQSMLIILQAYMIFINHYNGFSLLSVPIIKLIVTVAPNIIHLFITCYLYTNINHQQDSMNFALYSSDWTAMSINYKKMLLFTMRMNDAEKLKLKISLRKIVNLEMFASVMHLTYSIISVLAKSYGNTNTK

SEQ ID NO: 21 - protein, 424 aa, Acyrthosiphon pisum
MATGIKTVSKNEDNFMINMRLMKKTGFYQLLDSRSLKVFGHNVFKCMSVVQMSILSSVAFIFVANIYYFSDDINTVMMYSMLITSDVLSILKLYYILQNSDTIWNCIQMTSIDDLSYKYHDRRILEEGRSKSTSYSILIMFMWLNLIVSWSLGPLFVTNYFLIVEQNDEIYRYRFNIMNFAFPATDRFYNDNFMIYYGIEFITLVLWCHCTMNFDVLLLSMNITFKYQLKTISNSFSAFNFTRYNDFKNNRTKNVKHHKESESMFDFKSLIYDQQRVIENMKNIYRVFRPVVLTQLASESLIIMLLSCIIMLNYFNGISLLSALNLRIFAAISTFLFHIYVICYLFDDVNEQKDSMNLALYSSDWTTSDLQHKILLLHAMRMNNAENLRLQVTRNKIVNFQMFTYVRMIFFSFYYSGYCGHYFY

SEQ ID NO: 22 - protein, 437 aa, Acyrthosiphon pisum
MNPTFKHFFKGDCTNITKPSPMETCIDHTCTINLNILKQCGFYQIFDPNSKKIFGWNVYRISFIALTVITQCLIGFGNCGFLFELEDTTDNIDLFLIIFSNSYFCLTEWKVVILIINRKKFLELLDVTDLIFLKSKQCRKNIKILCKHRIRALQLTNLYFKFCIFVIIEWIIFPIMINSFIAHKTENRRLENVVNRRYPVDVNTYNKYYILFYVFEIIIGVKTVYLVLMVDILLLSIGWAIVIQYEVLAEAFKNIGYNENLQKDHDHDVDDYKYFKSILFDQQQLDSKVKLYFPIVKPIVLMHVAINSVLFIMLSNSFLMVFLSTESFTYKIVNLFKIGTGILYICLQLFLYCHLFDNINLKRKSVNLGIYSCNWTKMDLKFKKLLLLTMQINDANYITIKASTKTIVNLPIFANVLMTSYNIVSVMVKTMSKYRKT

SEQ ID NO: 23 - protein, 416 aa, Acyrthosiphon pisum
MKWLQDHEVAINLALFKRYQFYQIFNPNGSKLLNYDTYKLTNVMFIVAVTTYNIFSAMCFFTDTIDTIDSVDLLLMIFIYSIIIISLLKISVLLFNADQIWELFDLTRFDFLTSRQCRKNVGILCKYRDRSITITNLYQNYSTMVFIIWMITPLVLNTFVVVGGPNQRYHNIFNMQYPVSANIYNQYYYLFYLMEIAMGIFVLNYSMIVDNFLISLCWVIIAQYEVITTAFEKIGNDCELTTLQNEKNNNSFEAYEDLKSILMDQNKLYIKLKSFYRVVWIIVIFLIIIDSVLLIILTYSFVMICSSAESFSIFNILKISTAFFVFVIQLYLYCYLFDVLNDKKESVNFGLYCCDWTKMDLRFKKLLLLATKFNNANTLKIKSTPNKIVNLQLFSSVMTTAFNIVTVMLKTMNGKN

SEQ ID NO: 24 - protein, 424 aa, Acyrthosiphon pisum
MGIDNMSSLKSNEVAINLKLFKVFRFYHIFDPNSGKLCKFNVYHLAWYIINCVIGCILIYGLLGYFTEMEDVIDSIFHIQIMFCYLLYSLSLLKIITFLYKANNIWDLLRVTRINFLTSTQCQAHIGILHKHRNKSIKITNLISGFAIVTTLEWILFPLVLRLLSKTDASHSNKRFENIFNFRFPVTVCEYNNYYFIFYIMESFIAIFMLYAYVVTDVFFISVCYVIIAQYEIIKRAYEIVNCEQTSENNNENKNHNNIIVNDCCDDLISIVMDQQNHYAKLRLFYSTYKLIIVSTVVINSGSIIILTYASVVIFTSPETIPILSIVKLISAFTYMFFVLFFLCYLMECINNKIESVQLGMYSCNWTAMNIKSKKLLLFSMRMHNANKLMIKTTPNNIINLQLFNSVMMTSYNIVSAMVNTRSK

SEQ ID NO: 25 - protein, 426 aa, Acyrthosiphon pisum
MFSCDFINRTVNMNSENLFNGGSVAFNLSTYKQLGYYQLLDPKGPHIYGYHLYRTILKIFLLIVQFITIFGVMGFFIEMEDTDPGKSNSFELIIILTNCSLSSLKIYTLISNSKIIWDLFDLTRIDFLRCSRHSKLITKNFVKRCKKSTTITKWIARSFLVGLILWLMGPFIANEEHTEPNTVHRHKNIINIKFPVTMKTYNNYYFVFYLMEVAVGFCIVYGSVLIDAYLMSFCWIISAQYQSVTKAFATFGYNKQGSPKDIYKDFKSIIIDHQNIYLKMKSFYAVVRPITLIHVFAYSCSLIMYAYVIVTIFNSKELFIIAEIMKIVMTVSNVTMEVFIFCYLFELIDNKKEDVNFGLYSCNWTGMDIKFKQLLLMSMKMNNANRFKLKASPDVTINRPFFANVIHTCFKIVSVLIQTQSIDLLN

SEQ ID NO: 26 - protein, 427 aa, Acyrthosiphon pisum
MPNSSEECVMSSSMAKCTGLHYIIDPEGPTVGGHNVFHVTVMVMIGFTVVCLSMCPFGLYYWANDVTQCIFLLIYIVNFSFGCFKAFTLVRHSDDICRCLDVTRFDFSSGAIMSDPDSARFFRKCRDASSTFTGWFAASSHFVLLVWTLLPFVVVGKGVEINNRDGSTSYYHFNPYNMYFLVSSETYNRLHLVFHLVEWAFGLCFVLIMVAFDTFMVTLCVAITCQMRGIGNAYSKLGHDRCATASNVCSDGGIESNKSNNEYLRDLKLIIKDHQAVLGKMNDFYKIVGPVILPQLIVASFTIIFVSFIITRNYFNGMLLTSTQSLKMCCFPIFFYQVYYTCHAFGNLSHRKNVMNFALYSSDWTQMEIKFKKLLLLAMQMHDANKLDMKLTDKLVINLELFTRVINMCYSIFSVLVNSQLKIADKQ

SEQ ID NO: 27 - protein, 422 aa, Acyrthosiphon pisum
MDSKQEKQYIFNMKLARIMGLYQILFPNSTSFFGYNIYHVVTVFFVSFTFAISMLFPIGLLYLRNDIIAIMYYMGCISNFLLSCFKMVNILYHSKDIWKCIDVTSFNYILYKHYDRNVFKNWQTRSIRITYIYIVIALFAFFCWIFSPCIMNKSVIAIRNIDGSYSKYRMNIFNLYLIASNETYNKNFYIFYVIEIIISICYVYFTIVFDVLMLLVCFAISYQLETISNTIKSLGHEIYTRDNIRSGNSIKLKEKHGILYNDLITIMTDHQNVLKKLNDFYNIFRSITLTQIFIASSSHVFIWFIAAMSIDEGDNADSILSFKLFIVLPLINFQLFMTCSLFGTINEKKDSIIFALYSSNWTNMDLKSKKMILFNLTINNASQLKMKFTNTKIVNLEMFSHTMRFCYSIFSMLINYNKNKMK

SEQ ID NO: 28 - DNA, 29 bp, Artificial Sequence
  Synthetic - forward primer
cgcggcagcgctgggtacgatagaacatg

SEQ ID NO: 29 - DNA, 27 bp, Artificial Sequence
  Synthetic - reverse primer
cggcaccagcggatccggactcttgtc

SEQ ID NO: 30 - protein, 23 aa, Artificial Sequence
  Synthetic - pET45b His-Tag FASTA sequence
MAHHHHHHVGTGSNDDDDKSPDP

SEQ ID NO: 31 - protein, 22 aa, Artificial Sequence
  Synthetic - pNIC28-Bsa4 His-Tag FASTA sequence
MHHHHHHSSGVDLGTENLYFQS

SEQ ID NO: 32 - protein, 20 aa, Artificial Sequence
  Synthetic - pET15b His-Tag FASTA sequence
MGSSHHHHHHSSGLVPRGSH

SEQ ID NO: 33 - protein, 6 aa, Artificial Sequence
  Synthetic - pET45b cleavage site
DDDDKS

SEQ ID NO: 34 - protein, 7 aa, Artificial Sequence
  Synthetic - pNIC28-Bsa4 cleavage site
ENLYFQS

SEQ ID NO: 35 - protein, 7 aa, Artificial Sequence
  Synthetic - pET15b cleavage site
GLVPRGS

(19) United States
(12) Patent Application Publication
(10) Pub. No.: US 2023/0312476 A1
(43) Pub. Date: Oct. 5, 2023

(54) COMPOUNDS WITH SEMIOCHEMICAL PROPERTIES AND BIOSENSORS

(71) Applicant: ROTHAMSTED RESEARCH LIMITED, Harpenden Hertfordshire (GB)

(72) Inventors: Cassie Marie SIMS, Harpenden Hertfordshire (GB); Mike BIRKETT, Harpenden Hertfordshire (GB); David WITHALL, Harpenden Hertfordshire (GB); Alistair POXON, Harpenden Hertfordshire (GB)

(21) Appl. No.: 18/018,964

(22) PCT Filed: Jul. 30, 2021
(86) PCT No.: PCT/EP2021/071500

§ 371 Date: Jan. 31, 2023

(30) Foreign Application Priority Data
Jul. 31, 2020 (GB) 2011989.7

(51) Int. Cl. C07D 221/02; C07C 49/303; C07C 35/23; C07D 335/04; A01N 31/06; A01N 43/18; A01N 43/42

(52) U.S. Cl. CPC C07D 221/02; C07C 49/303; C07C 35/23; C07D 335/04; A01N 31/06; A01N 43/18; A01N 43/42; C07B 2200/05

(57) ABSTRACT

Provided are a compound of Formula I or Formula IA, a composition comprising a compound of Formula I or Formula IA, uses of the compounds and compositions to modulate insect behaviour and methods for modulating insect behaviour. Also provided are biosensors for detecting an analyte in a sample, uses of the biosensor, and methods for detecting an analyte in a sample.

### BACKGROUND OF THE INVENTION

### 1. Field of the Invention

Provided herein are compounds that exhibit semiochemical properties, in particular for insects, compositions comprising said compounds, uses of said compounds and compositions to modulate insect behaviour and methods for modulating insect behaviour. The invention further relates to biosensors for detecting an analyte in a sample, uses of said biosensor, and methods for detecting an analyte in a sample.

### 2. Related Art

A semiochemical is a compound that is secreted by organisms, which modifies the behaviour and/or development of another organism. Semiochemicals are categorised into intraspecific semiochemicals, pheromones, a compound or group of compounds that are released by an organism and induce a response in an individual of the same species, and interspecific semiochemicals, allelochemicals, which stimulate organisms of different species. Pheromones are critical for communication between insects of the same species; these may include sex pheromones, aggregation pheromones, and alarm pheromones.

Pheromones can be further categorised into releasers, pheromones which induces an immediate behavioural change, and primers, pheromones which initiate a complex set of physiological or developmental changes, but may result in no immediate behavioural change.

Semiochemicals are used in host location, mating and enemy warning systems.

Although semiochemicals are widely employed by insects for communication, communication chemistry and potential semiochemicals have been identified in many other organisms including mammals, birds and fish.

Aphid species, including the pea aphid, Acyrthosiphon pisum, have been shown to employ sex pheromones (e.g. (4aS,7S,7aR)-nepetalactone and (1R,4aS,7S,7aR)-nepetalactol) and an alarm pheromone, in addition to a range of allelochemistry generally utilised for host-location.

Semiochemicals have the potential to be used in pest management, by using mating disruption, pheromone traps, push-pull strategies and recruitment of natural enemies. Synthetic sex pheromone components have been used to catch male aphids and recruit foraging parasitic wasps, Aphidius ervi Haliday and Praon barbatum Mackauer, in the field. The synthetic sex pheromone components have also been found to attract other aphid natural predators, such as lacewings (Chrysopa cognata).

The alarm pheromone (E)-β-farnesene (EBF) has been shown to be repellent to aphids in behavioural studies, whilst attractive to natural enemy predators and parasitoids. A hexaploid commercial variety of wheat, Triticum aestivum cv. Cadenza (Poaceae), has been genetically engineered to biosynthesise and release EBF. Evaluation in field trials showed the transformed wheat variety was not significantly different from non-transformed varieties in managing aphids (assessed by aphid numbers and number of parasitized aphids), although controlled environment studies had demonstrated its effectiveness. This is likely due to the release rate of the alarm pheromone from the plant being consistent and steady, in contrast to a natural quick burst of pheromone produced by aphids.

Plant-derived semiochemicals also have practical applications; (Z)-jasmone has been found to be effective in reducing aphid numbers, attracting parasitic wasps and inducing the production of repellent volatiles in crops such as wheat, cotton and sweet peppers. (S)-Germacrene D has been identified as a potent repellent for aphids, but has little potential for commercial application in crop protection due to its chemical instability and cost of production. Development of more stable analogues which have comparable behavioural activity could be developed, particularly using modified terpene synthases and unnatural substrates.

Further advancement of the use of semiochemicals in pest management could stem from a deeper understanding of molecular recognition processes in the olfactory systems of pests. This may lead to ‘ab initio’ design of ligands, which may have similar behavioural effects as other olfactory ligands, but better prospects for commercial producibility. Currently, understanding of the olfactory system is limited, though two major groups of proteins are involved—olfactory receptors (ORs) and odorant binding proteins (OBPs)—both of which could provide potential pest management targets.

Given the significant value in understanding olfactory systems and semiochemicals in various industries, such as the food, health and pharmaceutical industries, there exists a need to develop new semiochemicals and sensors. The present invention addresses this need.

### SUMMARY

The invention relates generally to compounds that exhibit semiochemical properties, in particular for insects, and biosensors for detecting an analyte in a sample.

In one aspect of the invention, there is provided a compound of Formula I, or a salt, a solvate, a tautomer, a stereoisomer or a deuterated analogue thereof:

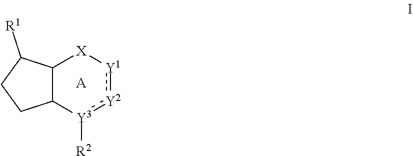

wherein,
X is C═O, C═S, C═S⁺—O—, C(R³)(R⁴), C═C(R⁵)(R⁶) or C═N(R⁷);
in ring A:
   (i) Y¹ is C(R⁸)(R⁹), S, S(O), S(O)₂ or N(R¹⁰), Y² is C(R¹¹)(R¹²) and Y³ is C(R¹¹); or
   (ii) Y¹ is C(R⁸)(R⁹), S, S(O), S(O)₂ or N(R¹⁰), Y² is C(R¹¹) and Y³ is C; or
   (iii) Y¹ is C(R⁸) or N, Y² is C(R¹¹) and Y³ is C(R¹¹);
R¹ is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R² is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R³ and R⁴ are independently selected from hydrogen, hydroxy, halogen, optionally substituted alkyl, optionally substituted alkoxy, optionally substituted alkanoyl and optionally substituted amino, or R³ and R⁴ together with the carbon atom to which they are attached form a 3-membered or 4-membered optionally substituted carbocyclic or optionally substituted heterocyclic ring;
R⁵ to R¹³ are independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; and
[symbol] represents a single or double bond to maintain correct atom valencies for Y¹, Y² and Y³ in ring A.

In another aspect of the invention, there is provided a compound of Formula IA, or a salt, a solvate, a tautomer, a stereoisomer or a deuterated analogue thereof:

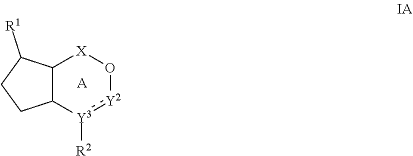

wherein,
X is C═O, C═S, C═S⁺—O—, C(R³)(R⁴), C═C(R⁵)(R⁶) or C═N(R⁷);
in ring A:
   (i) Y² is C(R¹¹)(R¹²) and Y³ is C(R¹³); or
   (ii) Y² is C(R¹¹) and Y³ is C;
R¹ is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R² is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R³ and R⁴ are independently selected from hydrogen, hydroxy, halogen, optionally substituted alkyl, optionally substituted alkoxy, optionally substituted alkanoyl and amino, or R³ and R⁴ together with the carbon atom to which they are attached form a 3-membered or 4-membered optionally substituted carbocyclic or optionally substituted heterocyclic ring;
R⁵ to R⁷ and R¹¹ to R¹³ are independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; and
[symbol] represents a single or double bond;

In one embodiment, the compound of Formula IA is not:

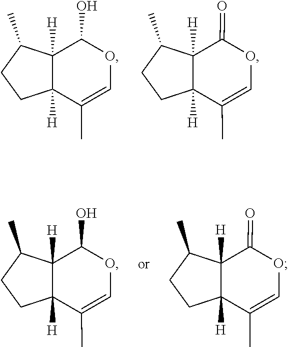

Preferably, in Formula IA, X may be C═S.

Preferably, in Formula IA, when Y² is C(R¹¹) and Y³ is C, R¹¹ may be a halogen atom.

Preferably, R¹ is C₁-C₆ alkyl or C₁-C₆ haloalkyl, especially C₁-C₆ alkyl.

More preferably, R¹ is methyl or trifluoromethyl.

Even more preferably, R¹ is methyl.

Preferably, R² is C₁-C₆ alkyl or C₁-C₆ haloalkyl, especially C₁-C₆ alkyl.

More preferably, R² is methyl or trifluoromethyl.

Even more preferably, R² is methyl.

Preferably, the compound has a structure according to any one of Formulae I-1 to I-7, IA-1 or IA-2:

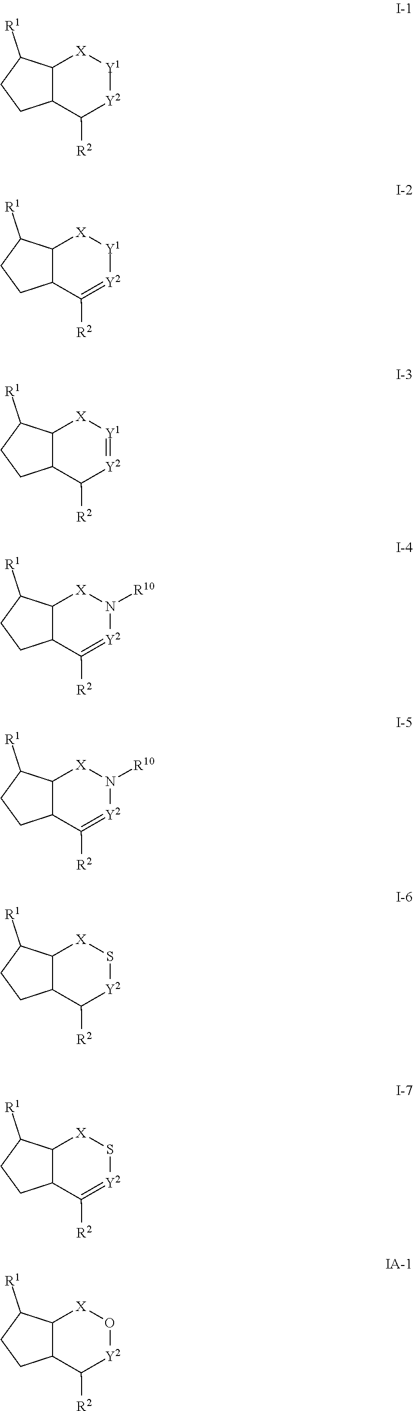

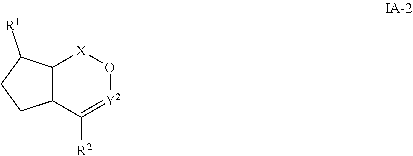

wherein X and R¹ to R¹², Y¹ and Y² are as defined as above.

Preferably, the compound has a structure according to any one of Formulae II-1 to II-7, IIA-1 or IIA-2, more preferably Formulae II-1 to II-3:

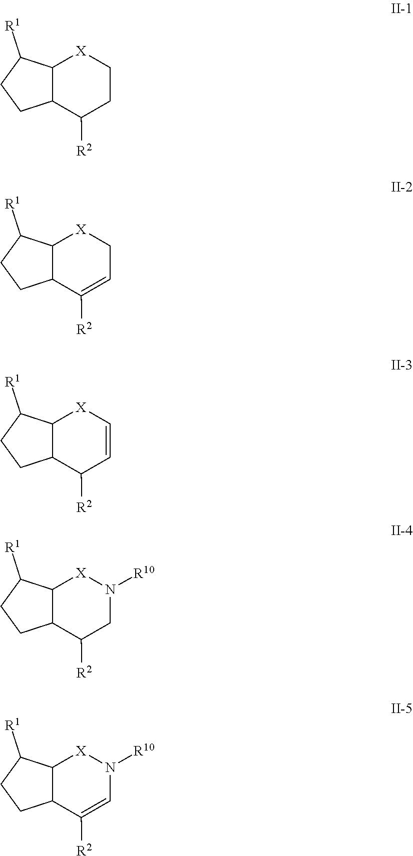

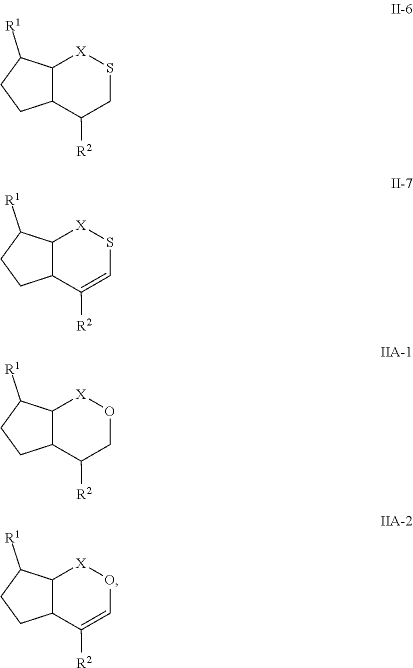

wherein X, R¹ to R⁷ are as defined as above.

Preferably, the compound has a structure according to any one of Formulae II-1-1 to II-1-16, more preferably Formulae II-1-1, II-1-4, II-1-5, II-1-8, II-1-9, II-1-12, II-1-13 and II-1-16, even more preferably Formulae II-1-1, II-1-5, II-1-9 and II-1-13:

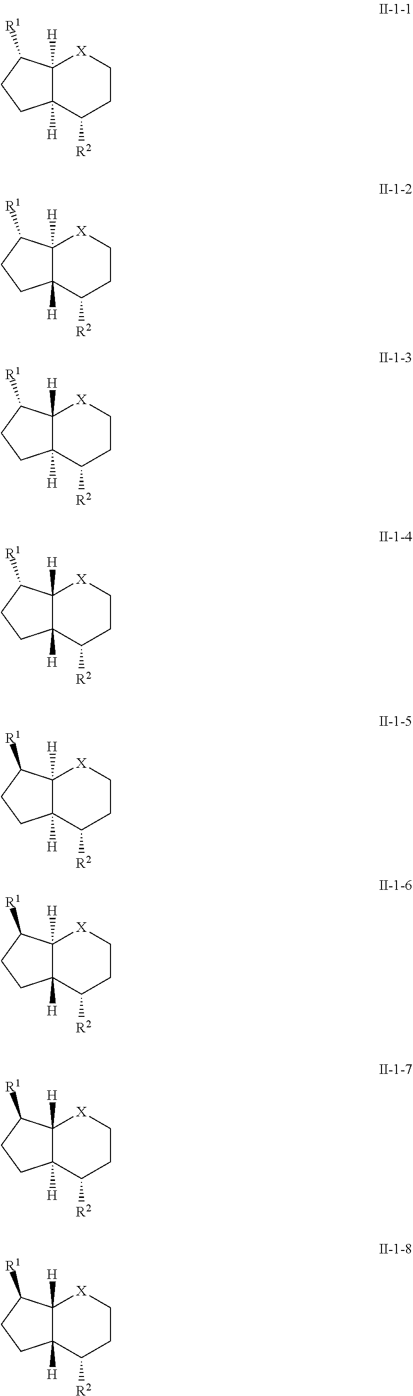

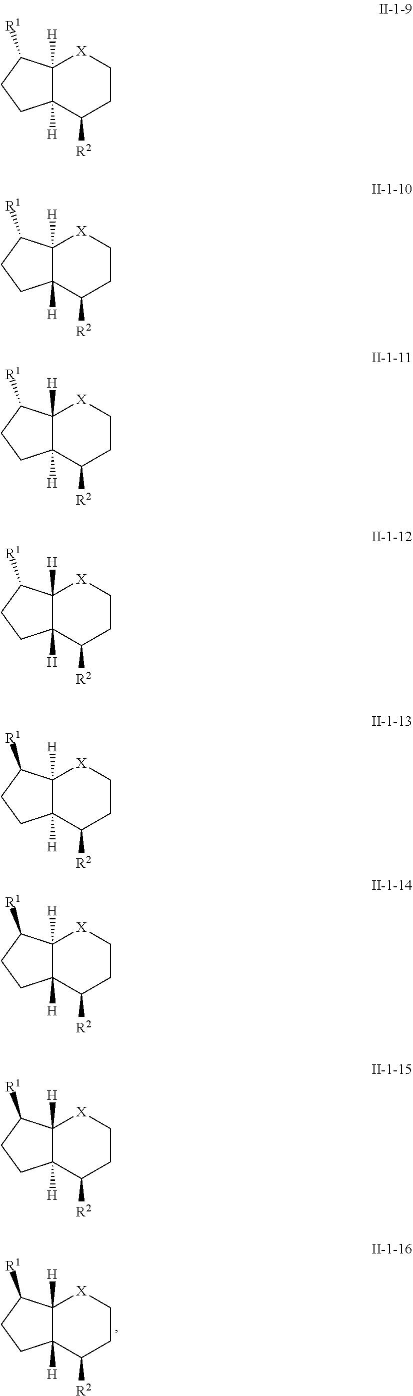

wherein X, R¹ to R⁷ are as defined as above.

Preferably, the compound has a structure according to any one of Formulae II-2-1 to II-2-8, more preferably Formulae II-2-1, II-2-4, II-2-5 and II-2-8, even more preferably Formulae II-2-1 and II-2-5:

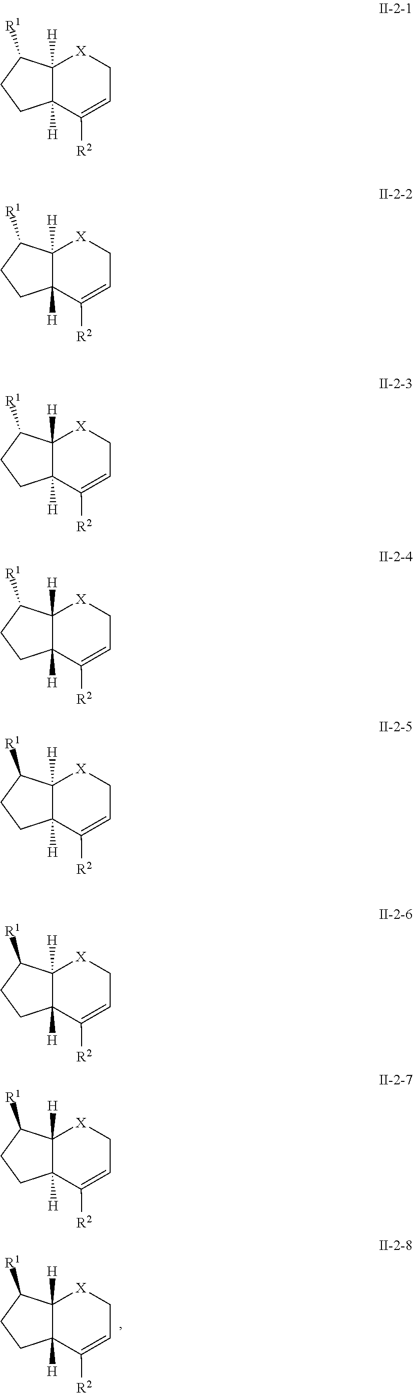

wherein X, R¹ to R⁷ are as defined as above.

Preferably, the compound has a structure according to any one of Formulae II-3-1 to II-3-16, more preferably Formulae II-3-1, II-3-4, II-3-5, II-3-8, II-3-9, II-3-12, II-3-13 and II-3-16, even more preferably Formulae II-3-1, II-3-5, II-3-9 and II-3-13:

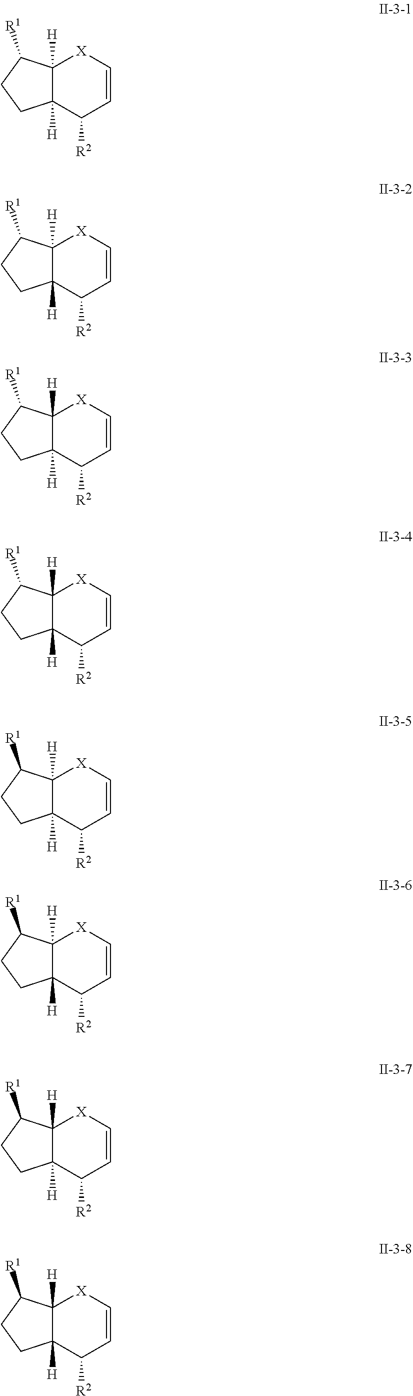

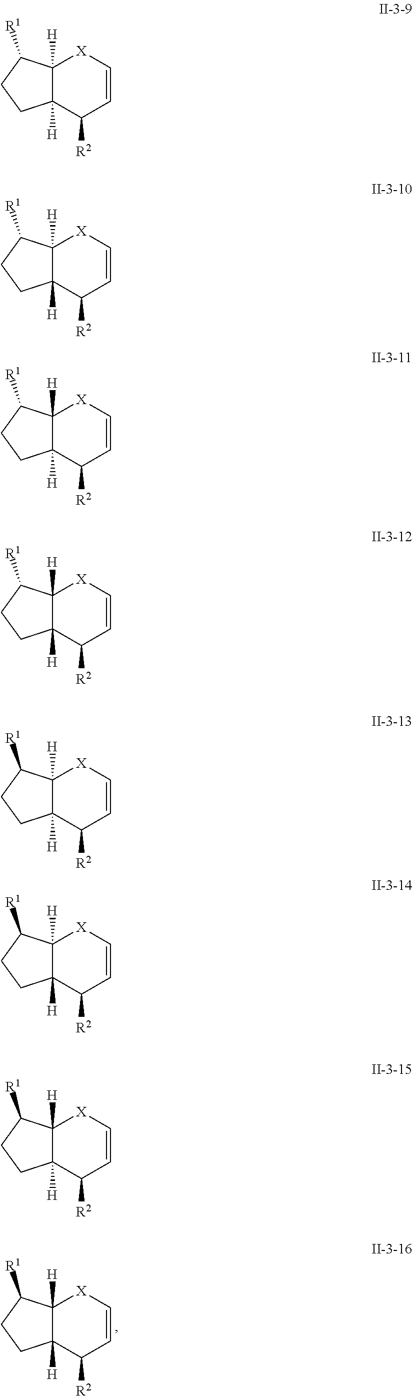

wherein X, R¹ to R⁷ are as defined as above.

Preferably, the compound has a structure according to any one of Formulae II-4-1 to II-4-16, more preferably Formulae II-4-1, II-4-4, II-4-5, II-4-8, II-4-9, II-4-12, II-4-13 and II-4-16, even more preferably Formulae II-4-1, II-4-5, II-4-9 and II-4-13:

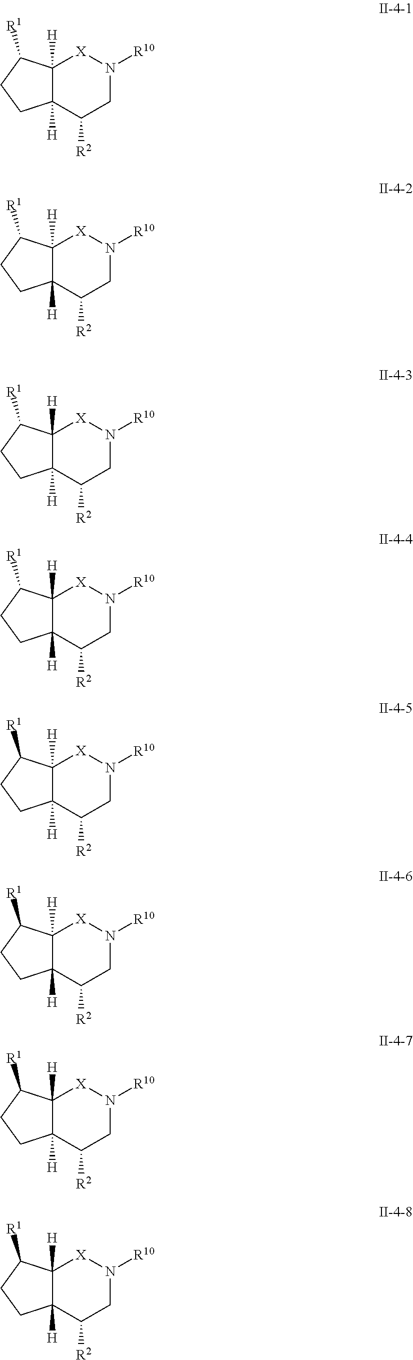

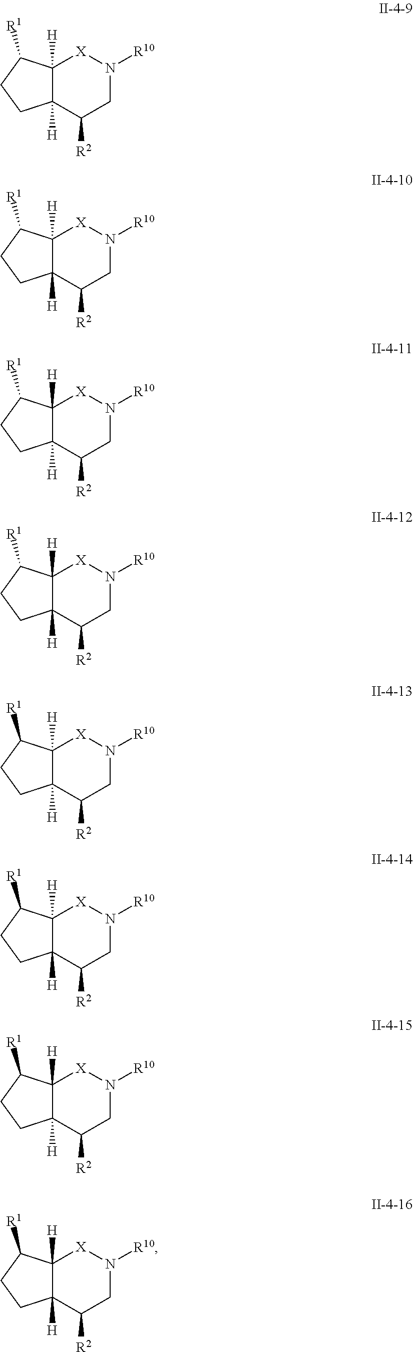

wherein X, R¹ to R⁷ and R¹⁰ are as defined as above.

Preferably, the compound has a structure according to any one of Formulae II-5-1 to II-5-8, more preferably II-5-1, II-5-4, II-5-5 and II-5-8, even more preferably Formulae II-5-1 and II-5-5:

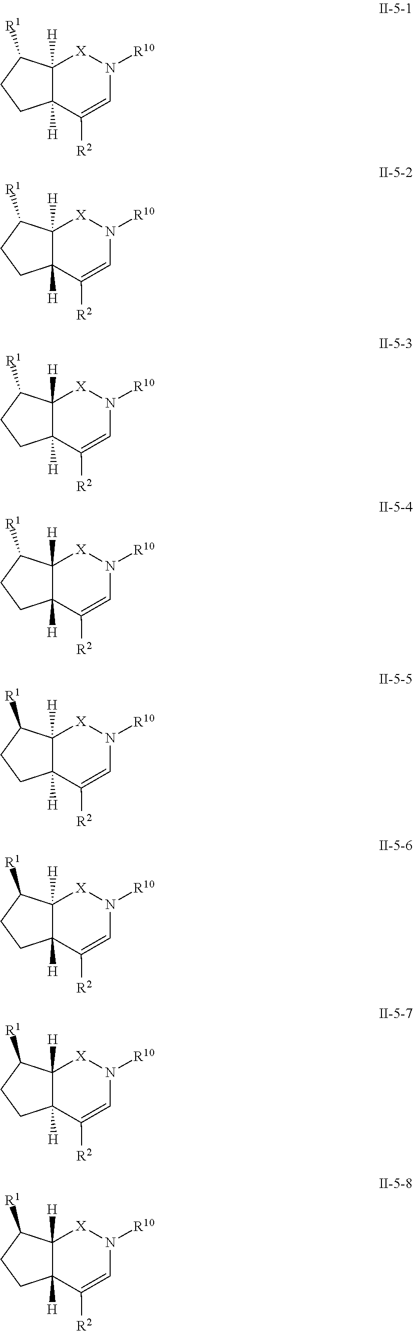

wherein X, R¹ to R⁷ and R¹⁰ are as defined as above.

Preferably, the compound has a structure according to any one of Formulae II-6-1 to II-6-16, more preferably Formulae II-6-1, II-6-4, II-6-5, II-6-8, II-6-9, II-6-12, II-6-13 and II-6-16, even more preferably Formulae II-6-1, II-6-5, II-6-9 and II-6-13:

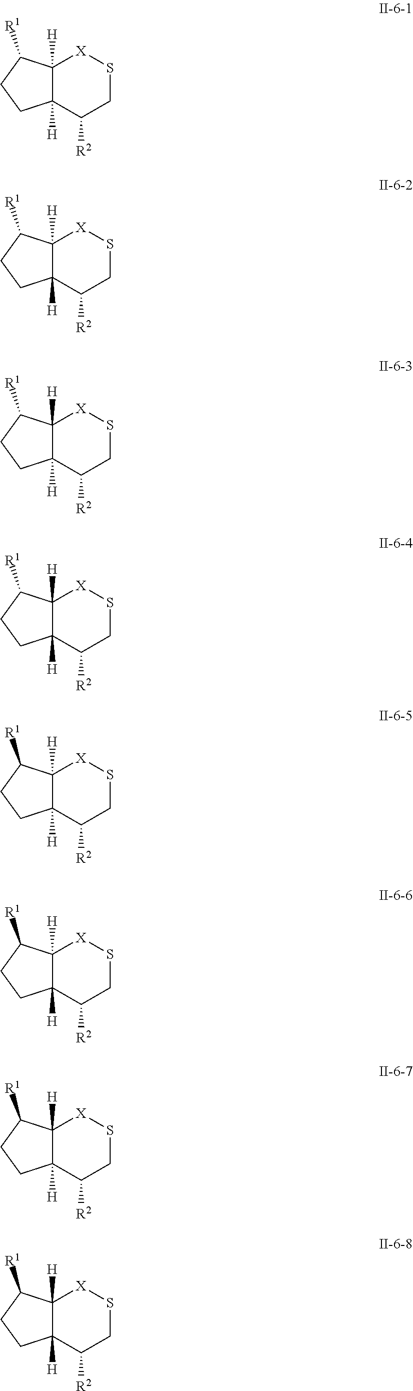

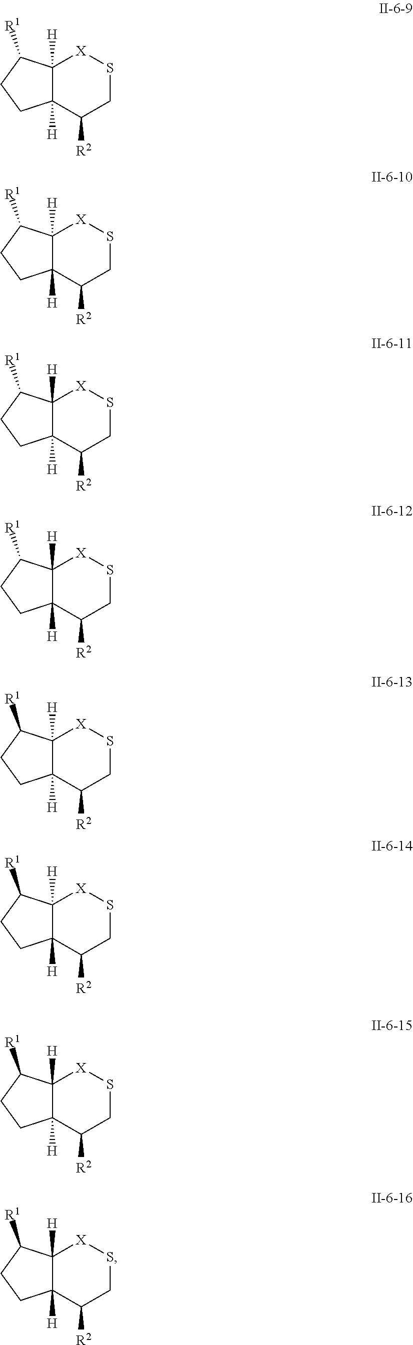

wherein X, R¹ to R⁷ are as defined as above.

Preferably, the compound has a structure according to any one of Formulae II-7-1 to II-7-8, more preferably Formulae II-7-1, II-7-4, II-7-5 and II-7-8, even more preferably Formulae II-7-1 and II-7-5:

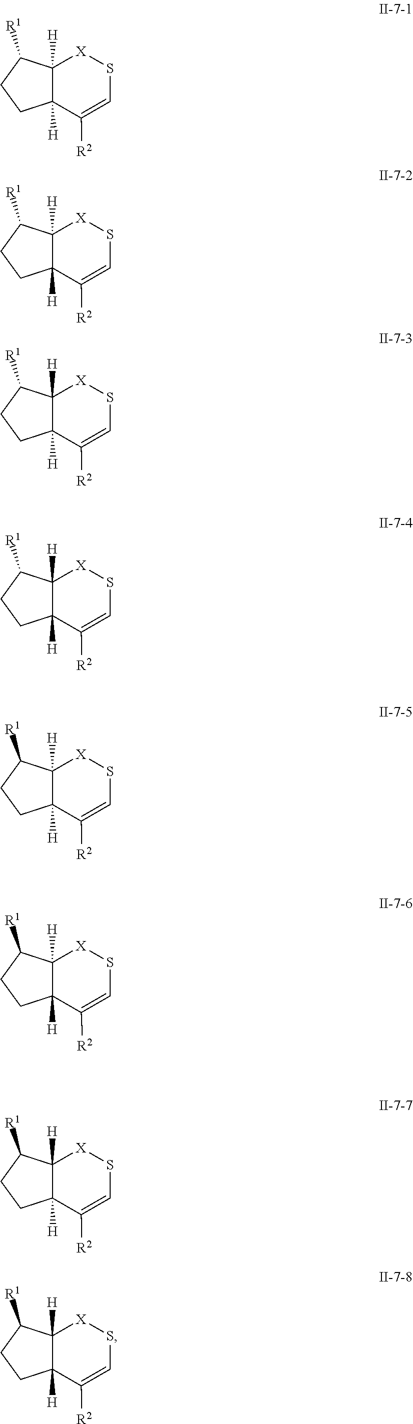

wherein X, R¹ to R⁷ are as defined as above.

Preferably, the compound has a structure according to any one of Formulae IIA-1-1 to IIA-1-16, preferably Formulae IIA-1-1, IIA-1-4, IIA-1-5, IIA-1-8, IIA-1-9, IIA-1-12, IIA-1-13 and IIA-1-16, even more preferably Formulae IIA-1-1, IIA-1-5, IIA-1-9 and IIA-1-13:

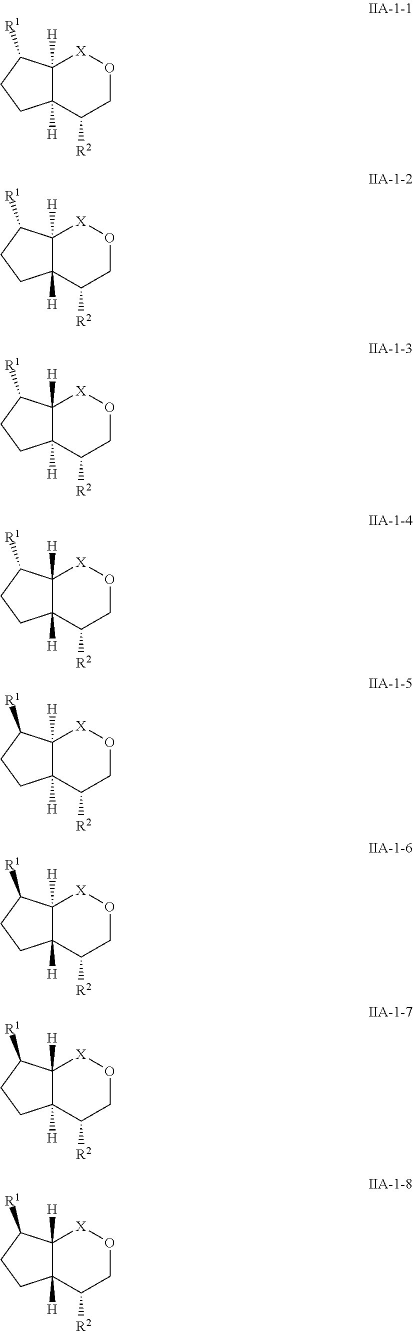

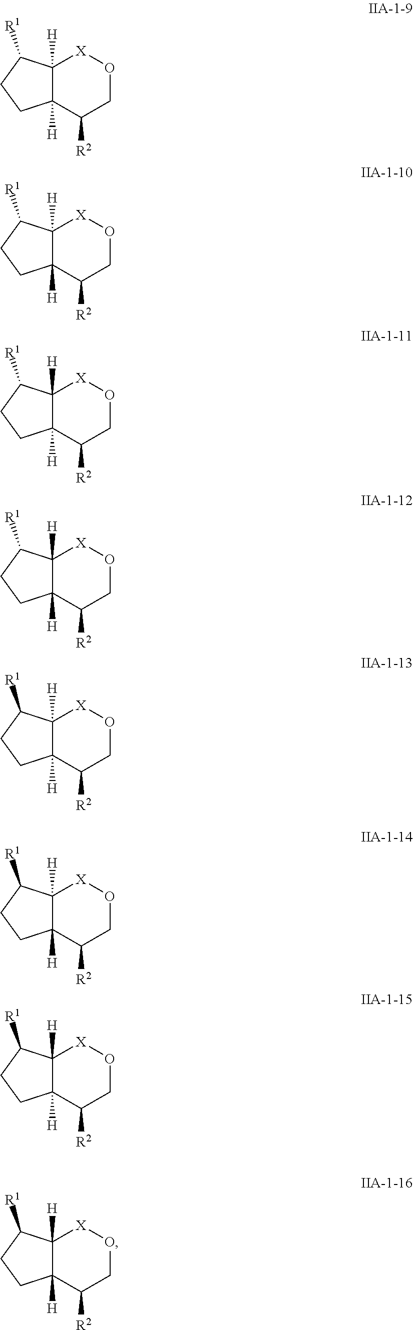

wherein X, R¹ to R⁷ are as defined as above.

Preferably, the compound has a structure according to any one of Formulae IIA-1-1 to IIA-2-8, more preferably Formulae IIA-2-1, IIA-2-4, IIA-2-5 and IIA-2-8, even more preferably Formulae IIA-2-1 and IIA-2-5:

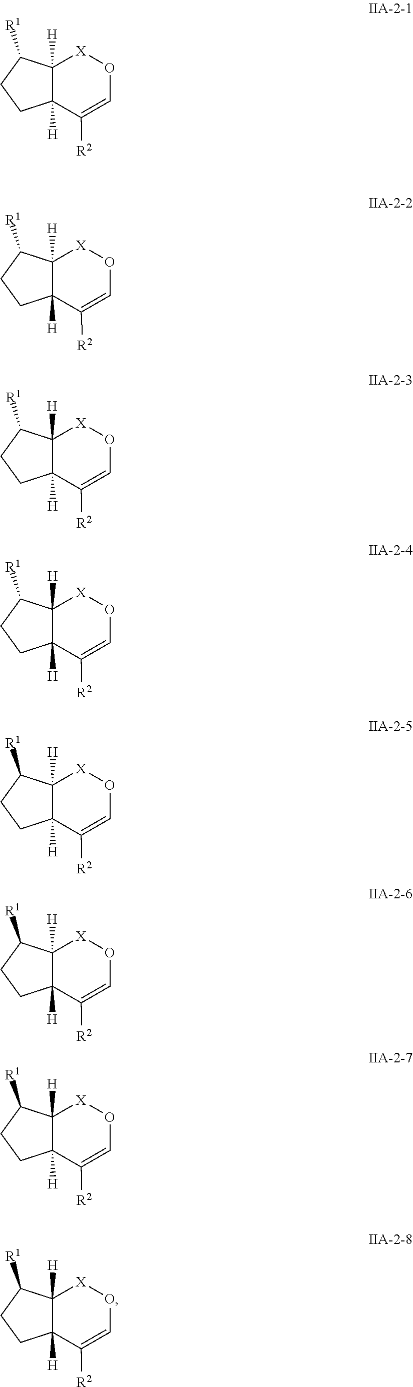

wherein X, R¹ to R⁷ are as defined as above.

Preferably, X is C═O, C═S, CH(OH) or CH(NH₂).

More preferably, X is C═O, CH(OH) or CH(NH₂).

Preferably, R⁸ is hydrogen or halogen, and/or wherein R⁹ is hydrogen or halogen.

Preferably, R¹⁰ is hydrogen, hydroxyl or C₁-C₆ alkyl.

Preferably, R¹¹ is hydrogen or halogen.

Preferably, the compound has a structure according to any one of Formulae III-1 to III-22, more preferably III-1, III-5, III-7, III-8, III-9, III-10, III-11, III-12, III-15, III-16, III-18, III-19 and III-22, even more preferably Formulae III-1, III-8, III-9, III-11, III-12 and III-16:

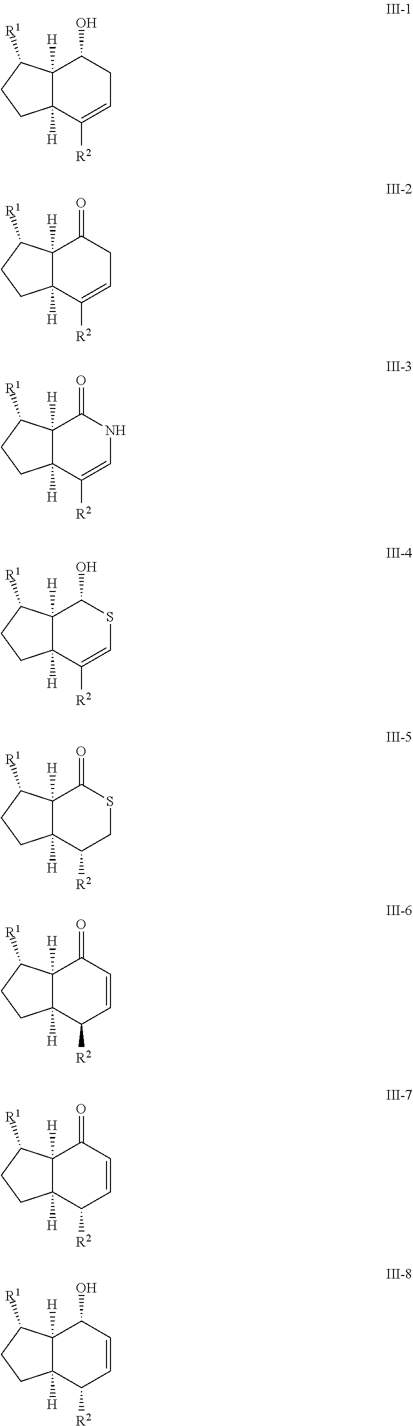

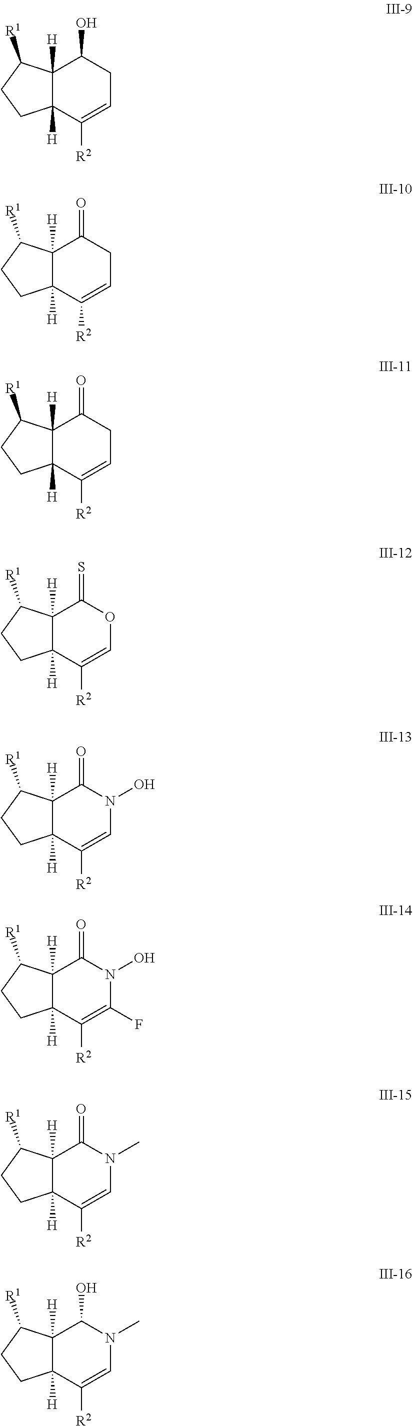

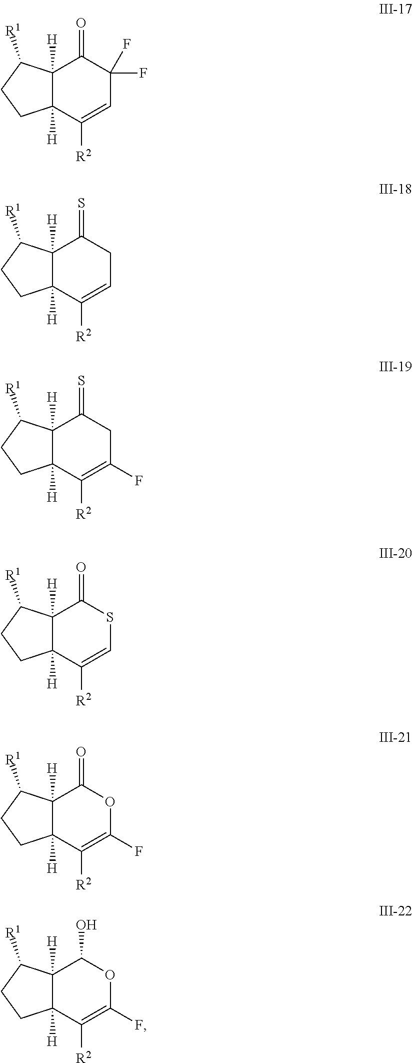

wherein R¹ and R² are as defined as above.

Preferably, the compound has a structure according to any one of Formulae III-1, III-2 and III-4 to III-11.

More preferably, the compound may have a structure according to any one of Formulae III-1, III-5, III-7, III-8, III-9, III-10 and III-11.

Even more preferably, the compound may have a structure according to any one of Formulae III-1, III-8, III-9 and III-11.

Preferably, the compound is selected from any one of the following:

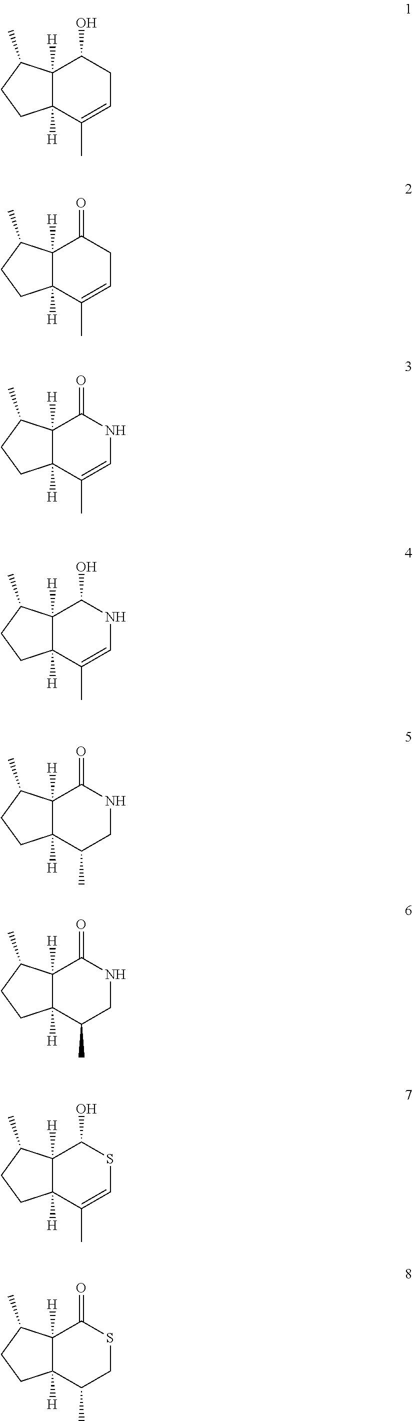

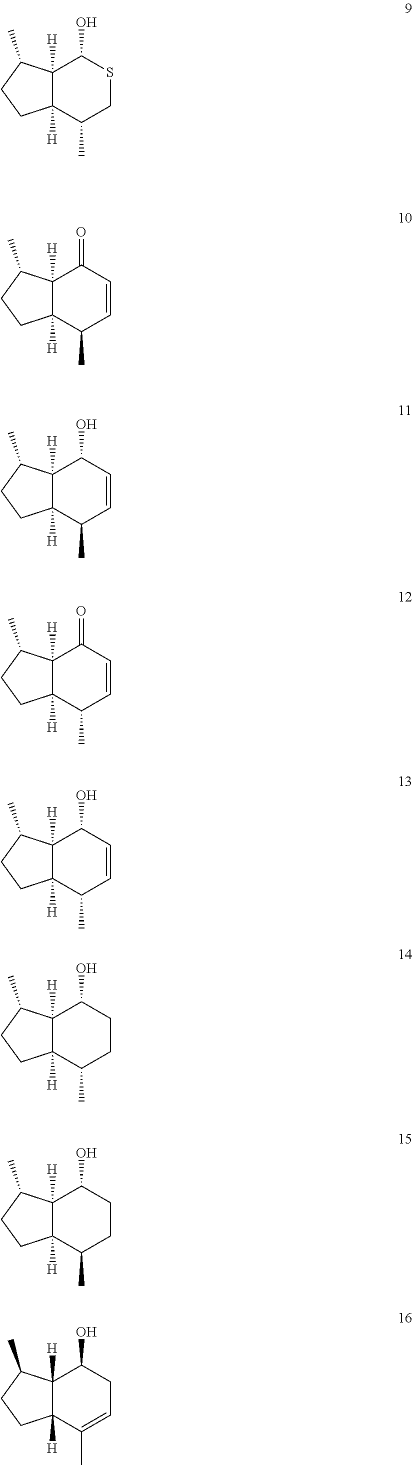

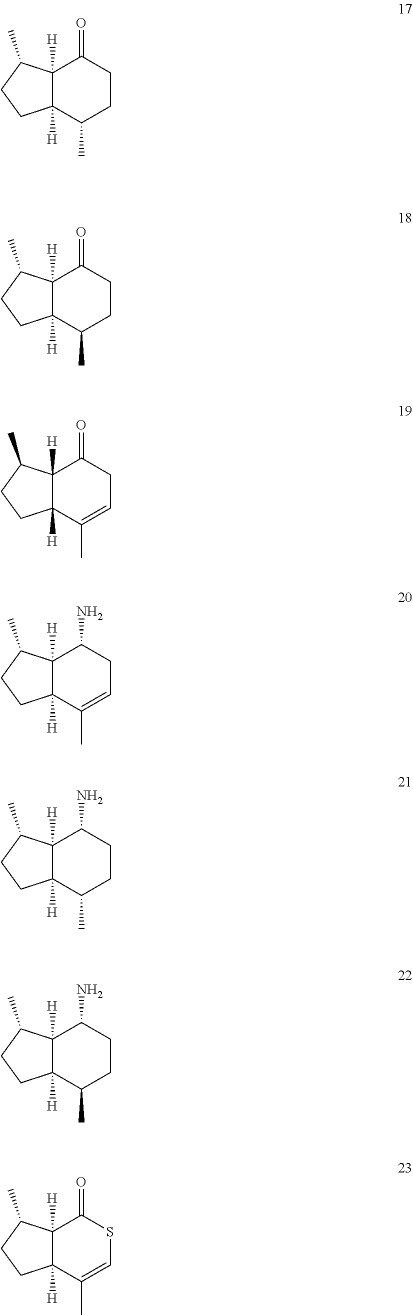

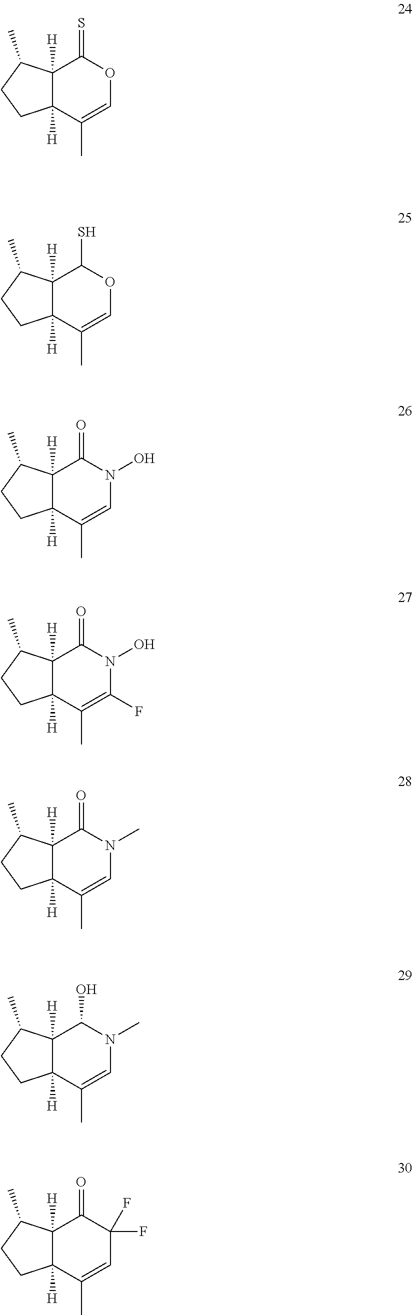

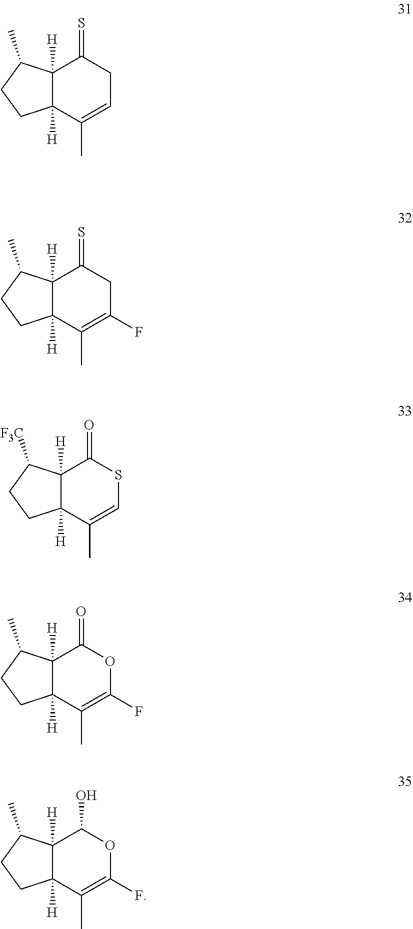

Preferably, the compound is selected from any one of Compounds 1, 2, 3, 7, 8, 10, 12, 13, 16, 17, 19, 24 and 26-35.

More preferably, the compound is selected from any one of Compounds 1, 8, 12, 13, 16, 17, 19, 24, 28, 29, 31, 32 and 35.

Even more preferably, the compound is selected from any one of Compounds 1, 13, 16, 19, 24 and 29.

Preferably, the compound is selected from any one of Compounds 1-22.

More preferably, the compound is selected from any one of Compounds 1, 2, 7, 8, 10, 12, 13, 16, 17 and 19.

Even more preferably, the compound is selected from any one of Compounds Compounds 1, 8, 12, 13, 16, 17 and 19.

Yet even more preferably, the compound is selected from any one of Compounds 1, 13, 16 and 19.

In another aspect of the invention, there is provided a composition comprising a compound as defined above and a carrier.

Preferably, the carrier is an agrochemically or dermatologically acceptable carrier.

It is preferred that the agrochemically acceptable carrier is selected from natural and synthetic clays and silicates, natural silicas, diatomaceous earths, magnesium silicates, talcs, magnesium aluminium silicates, attapulgites, vermiculites, aluminium silicates, kaolinites, montmorillonites, micas, calcium carbonate, calcium sulfate, ammonium sulfate, synthetic hydrated silicon oxides, synthetic calcium or aluminium silicates, carbon, sulfur, natural and synthetic resins, coumarone resins, polyvinyl chloride, styrene polymers and copolymers, solid polychlorophenols, bitumen, waxes, solid fertilisers, superphosphates, water, alcohols, isopropanol, glycols, ketones, acetone, methyl ethyl ketone, methyl isobutyl ketone, cyclohexanone, ethers, aromatic and araliphatic hydrocarbons, benzene, toluene, xylene, petroleum fractions, kerosene, light mineral oils, chlorinated hydrocarbons, carbon tetrachloride, perchloroethylene and trichloroethane.

Preferably, the dermatologically acceptable carrier is selected from silicone, petrolatum, lanolin, liquid hydrocarbons, agricultural spray oils, paraffin oil, tall oils, liquid terpene hydrocarbons and terpene alcohols, aliphatic and aromatic alcohols, esters, aldehydes, ketones, mineral oil, higher alcohols, finely divided organic and inorganic solid materials.

Preferably, the composition further comprises at least one additional active ingredient.

More preferably, the additional active ingredient is an insecticide or insect repellent.

It is preferred that the insecticide is selected from aldrin, chlordane, chlordecone, DDT, dieldrin, endosulfan, endrin, heptachlor, hexachlorobenzene, lindane, methoxychlor, mirex, pentachlorophenol, dichlorodiphenyldichloroethane, acephate, azinphos-methyl, bensulide, chlorethoxyfos, chlorpyrifos, chlorpyrifos-methyl, diazinon, dichlorvos, dicrotophos, dimethoate, disulfoton, ethoprop, fenamiphos, fenitrothion, fenthion, fosthiazate, malathion, methamidophos, methidathion, mevinphos, monocrotophos, naled, omethoate, oxydemeton-methyl, parathion, parathion-methyl, phorate, phosalone, phosmet, phostebupirim, phoxim, pirimiphos-methyl, profenofos, terbufos, tetrachlorvinphos, tribufos, trichlorfon, aldicarb, bendiocarb, carbofuran, carbaryl, dioxacarb, fenobucarb, fenoxycarb, isoprocarb, methomyl, oxamyl, propoxur, 2-(1-methylpropyl)phenyl methylcarbamate, allethrin, bifenthrin, cyhalothrin, cypermethrin, cyfluthrin, deltamethrin, etofenprox, fenvalerate, permethrin, phenothrin, prallethrin, resmethrin, tetramethrin, tralomethrin, transfluthrin, acetamiprid, clothianidin, dinotefuran, imidacloprid, nithiazine, thiacloprid, thiamethoxam, chlorantraniliprole, cyantraniliprole, flubendiamide, diflubenzuron, flufenoxuron, cyromazine, methoprene, hydroprene, tebufenozide, anabasine, anethole, annonins, pawpaw tree seeds, azadirachtin, caffeine, Carapa, cinnamaldehyde, cinnamon leaf oil, cinnamyl acetate, citral, deguelin, Derris, Desmodium caudatum, eugenol, ivermectin, linalool, myristicin, Neem oil, nicotine, Peganum harmala, oregano oil, Quassia, ryanodine, rotenone, spinosad, spinosyn A, spinosyn D, tetranortriterpenoid, thymol, Bacillus sphaericus, Bacillus thuringiensis, Bacillus thuringiensis aizawi, Bacillus thuringiensis israelensis, Bacillus thuringiensis kurstaki, Bacillus thuringiensis tenebrionis, nuclear polyhedrosis virus, granulovirus, Lecanicillium lecanii, diatomaceous earth, borax and boric acid.

Preferably, the insect repellent is selected from methyl anthranilate, benzaldehyde, N,N-diethyl-m-toluamide, dimethyl carbate, dimethyl phthalate, ethylhexanediol, icaridin, butopyronoxyl, ethyl butylacetylaminopropionate, metofluthrin, SS220, tricyclodecenyl allyl ether, VUAA1, Callicarpa, birch tree bark, Myrica gale, catnip oil, citronella oil, eucalyptus oil, lemon eucalyptus essential oil, p-menthane-3,8-diol, Neem oil, nepetalactone, nepetalactol, lemongrass, tea tree oil, tobacco, Achillea alpine, alpha-terpinene, basil, sweet basil, breadfruit, camphor, carvacrol, castor oil, cedar oil, celery extract, cinnamon, oil of cloves, fennel oil, garlic, geranium oil, lavender, marigold, marjoram, mint, menthol, oleic acid, Mentha pulegium, peppermint, rosemary, Lantana camara, thyme, yellow nightshade and Andrographis paniculata.

In another aspect of the invention, there is provided use of a compound or composition as defined above to modulate insect behaviour.

Preferably, the use is as an insect repellent, insect attractant or insect mating disruptant.

Preferably, the insect is selected from aphids, lacewings, houseflies, mosquitoes, cockroaches, mites and ticks.

More preferably, the insect is an aphid.

Even more preferably, the aphid is Acyrthosiphon pisum.

In another aspect of the invention, there is provided a method of modulating insect behaviour, wherein a compound or composition as defined above is applied to a locus.

Preferably, the locus is a plant or a part thereof.

Preferably, the locus is an insect trap.

Preferably, the compound or composition acts as an insect repellent, insect attractant or insect mating disruptant.

Preferably, the insect is selected from aphids, lacewings, houseflies, mosquitoes, cockroaches, mites and ticks.

More preferably, the insect is an aphid.

Even more preferably, the aphid is Acyrthosiphon pisum.

In another aspect of the invention, there is provided a biosensor, the biosensor comprising:
a protein having an amino acid sequence as defined in SEQ ID NO: 6, or a fragment or variant thereof and
a signal generator, wherein the signal generator is configured to output a signal when the analyte is bound to the protein.

Preferably, the biosensor further comprises:
a flow path for moving the sample;
a substrate; and
a protein-containing layer immobilised to the substrate and in contact with the flow path, wherein the protein-containing layer comprises the protein.

In another aspect of the invention, there is provided use of the biosensor, wherein the biosensor is used to identify olfactory ligands.

In another aspect of the invention, there is provided use of the biosensor, wherein the biosensor is used in high-throughput screening.

In another aspect of the invention, there is provided use of the biosensor, wherein the biosensor is used to detect field populations of aphids.

In another aspect of the invention, there is provided a method for detecting an analyte in a sample, the method comprising:
a. providing a biosensor as defined above;
b. contacting the biosensor with the sample; and
c. comparing a magnitude of the signal generated by the biosensor when the sample is present with a reference magnitude of the signal generated by the biosensor when the sample is absent.

Preferably, the biosensor is used to identify olfactory ligands.

Preferably, the biosensor is used in high-throughput screening.

Preferably, the biosensor is used to detect field populations of aphids.

### BRIEF DESCRIPTION OF THE DRAWINGS

The invention is further described by reference to the following non-limiting figures.

FIG. 1 shows homology models of A. pisum odorant binding proteins (OBP1-OBP11).

FIG. 2 shows: (i) predicted structure of OBP6 (green) with potential binding pockets shown in purple; (ii) potential salt bridge between Lys144 and Asp174; (iii) predicted structure of OBP6 with the adaptive Poisson-Boltzmann Solver (APBS) electrostatic map displayed.

FIG. 3 shows results from Autodock ligand-screening. The aphid sex pheromone components and respective enantiomers were tested, along with the aphid alarm pheromone.

FIG. 4 shows predicted binding interactions (shown as 1/Kᵢ) of the aphid sex pheromone components with OBPs 6-9. The natural enantiomers are indicated with an asterisk.

FIG. 5 shows: (i) OBP6 (green) interactions with the enantiomers of the sex pheromone; naturally occurring (1R,4aS,7S,7aR)-nepetalactol (pink) interacting with Tyr176 and Phe208 (red, dashed); (ii) non-naturally occurring (1S,4aR,7R,7aS)-nepetalactol (blue) with a potential interaction with Lys169 (red, dashed). Distance of interactions are in Å.

FIG. 6 shows: (i) the general transmembrane domains of insect odorant receptors; (ii) homology models of Acyrthosiphon pisum odorant-receptor 5 embedded in a lipid bilayer.

FIG. 7 shows tryptophan fluorescence of OBP6 at 2 μM with 2 μM 1-NPN titrated with various ligands to final concentrations of 0-22 The lowest concentration of each ligand (0 μM) can be seen in red and the highest concentration (22 μM) in black. For each ligand, only one data set is presented for clarity.

FIG. 8 shows: (i) binding curves of OBP6 with the aphid sex pheromone components (black) and their respective enantiomers (red); (ii) binding curves of OBP6 with the aphid alarm pheromone (blue) and linalool (purple); (iii) calculated K_{D} values of OBP6 with various ligands.

FIG. 9 shows the calculated K_{D} values for OBP6 and various ligands from fluorescent binding studies versus the predicted K_{D}s from in silico testing.

FIG. 10 shows results from Autodock ligand-screening. The aphid sex pheromone components and respective enantiomers were tested, along with the aphid alarm pheromone.

FIG. 11 shows the saturation transfer difference (STD) nuclear magnetic resonance (NMR) spectra of bovine serum albumin (BSA) with tryptophan (7.00-8.00 ppm) and sucrose (3.00-4.40 ppm). The initial ¹H can be seen (top spectrum), in addition to the STD NMR (bottom spectrum), where only tryptophan peaks remain.

FIG. 12 shows the STD NMR spectra of A. pisum OBP6 with (4aS,7S,7aR)-nepetalactone. The initial ¹H spectra (top spectrum), in addition to the STD NMR (bottom spectrum).

FIG. 13 shows: (i) assignments and changes in relative intensity of different peaks in the STD NMR spectrum of OBP6 and (4aS,7S,7aR)-nepetalactone; (ii) STD absolute values and changes in relative intensity of different protons in the STD NMR spectrum of OBP6 and (4aS,7S,7aR)-nepetalactone; (iii) the structure of (4aS,7S,7aR)-nepetalactone annotated with the epitope mapping results.

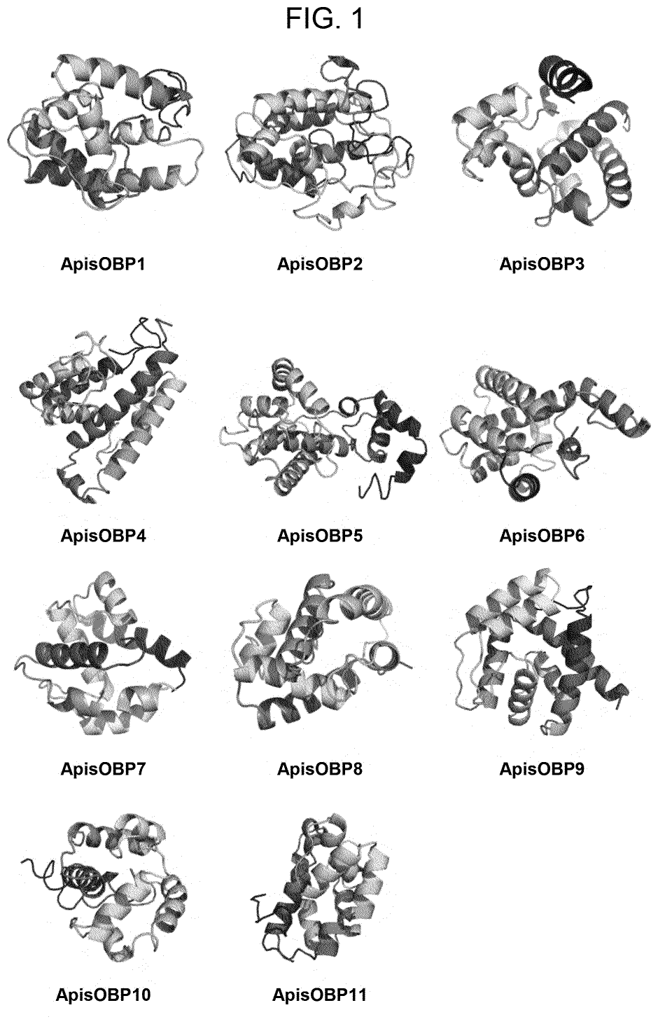

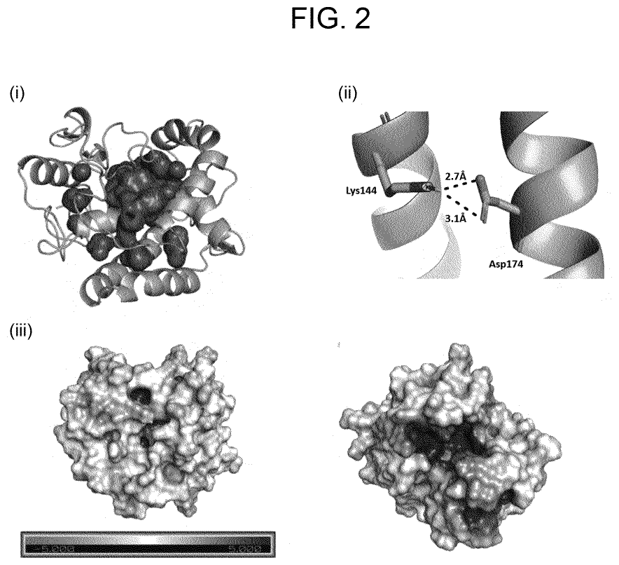

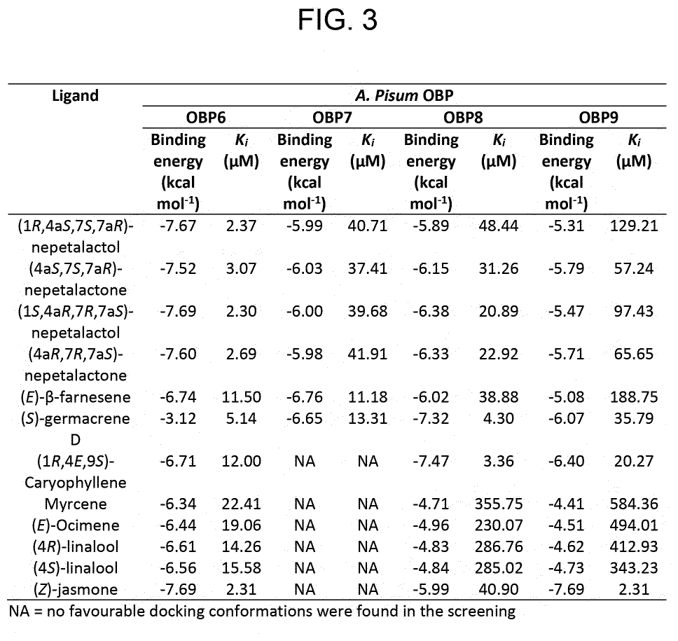

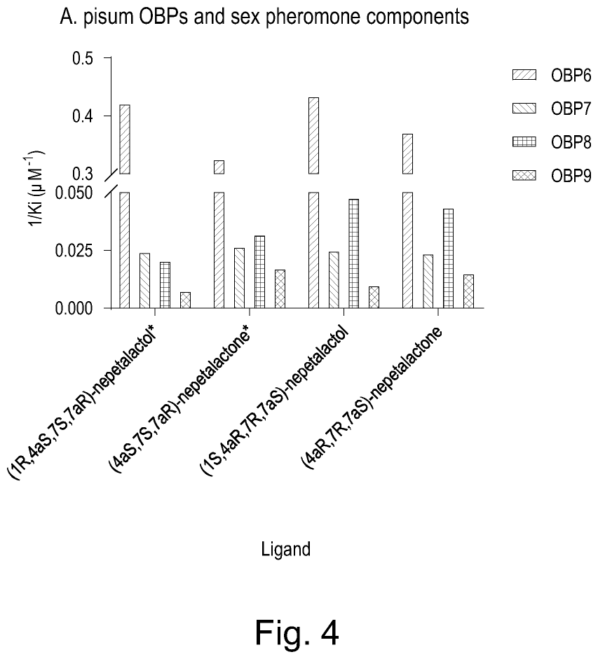

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of microbiology, tissue culture, molecular biology, chemistry, biochemistry, recombinant DNA technology and biosensor fabrication, which are generally known in the art. Such techniques are explained fully in the literature.

### 3. General Chemical Definitions

The term “hydroxyl” or “hydroxy” as used herein refers to the group —OH.

The term “halo” or “halogen” as used herein refers to any radical of fluorine, chlorine, bromine or iodine.

The term “cyano” as used herein refers to the group —CN.

The term “alkyl” as used herein, by itself or as part of another group, refers to both straight and branched chain radicals of up to twelve carbons. For example, an alkyl group may contain 1, 2, 3, 4, 5, 6, 7, 8, 9, 10, 11 or 12 carbon atoms. Non-limiting examples of C₁-C₁₂ alkyl groups include methyl, ethyl, propyl, isopropyl, butyl, sec-butyl, tert-butyl, 3-pentyl, hexyl and octyl groups. Preferably, the term “alkyl” as used herein, by itself or as part of another group, may refer to a straight or branched chain radical comprising from one to eight carbon atoms, more preferably one to six carbon atoms and even more preferably one to four carbon atoms. An “optionally substituted alkyl” group may include the substituents as described below for the term “optionally substituted”.

For example, an “optionally substituted alkyl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted alkyl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “haloalkyl” as used herein, by itself or as part of another group, refers to both straight and branched chain radicals of up to twelve carbon atoms, comprising at least one halogen atom. For example, a haloalkyl group may contain 1, 2, 3, 4, 5, 6, 7, 8, 9, 10, 11 or 12 carbon atoms. Preferably, the term “haloalkyl” as used herein, by itself or as part of another group, may refer to a straight or branched chain radical comprising from one to eight carbon atoms, more preferably one to six carbon atoms and even more preferably one to four carbon atoms, and comprising at least one halogen atom.

For example, a “haloalkyl” group may be a fluoroalkyl or perfluoroalkyl group.

Preferably, a “haloalkyl” group may be a C₁-C₆ fluoroalkyl group, or a C₁-C₆ perfluoroalkyl group.

Even more preferably, a “haloalkyl” group may be a C₁-C₄ fluoroalkyl group, or a C₁-C₄ perfluoroalkyl group. For example, a “haloalkyl” group may include difluoromethyl, trifluoromethyl or pentafluoroethyl.

The term “alkenyl” as used herein, by itself or as part of another group, refers to both straight and branched chain radicals of up to twelve carbons, and which comprise at least one carbon-carbon double bond. For example, an alkenyl group may contain 2, 3, 4, 5, 6, 7, 8, 9, 10, 11 or 12 carbon atoms. Preferably, the term “alkenyl” as used herein, by itself or as part of another group, may refer to a straight or branched chain radical comprising from one to eight carbon atoms, more preferably one to six carbon atoms and even more preferably one to four carbon atoms, and which comprise at least one carbon-carbon double bond. An “optionally substituted alkenyl” group may include the substituents as described below for the term “optionally substituted”.

For example, an “optionally substituted alkenyl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted alkenyl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

More preferably, an “optionally substituted alkenyl” group may include a C₂-C₆ fluoroalkenyl group, or a C₂-C₆ perfluoroalkenyl group.

Even more preferably, an “optionally substituted alkenyl” group may include a C₂-C₄ fluoroalkenyl group, or a C₂-C₄ perfluoroalkenyl group.

The term “alkynyl” as used herein, by itself or as part of another group, refers to both straight and branched chain radicals of up to twelve carbons, and which comprise at least one carbon-carbon triple bond. For example, an alkynyl group may contain 2, 3, 4, 5, 6, 7, 8, 9, 10, 11 or 12 carbon atoms. For example, the term “alkynyl” as used herein, by itself or as part of another group, may refer to a straight or branched chain radical comprising from one to eight carbon atoms, more preferably one to six carbon atoms and even more preferably one to four carbon atoms, and which comprise at least one carbon-carbon triple bond. An “optionally substituted alkynyl” group may include the substituents as described below for the term “optionally substituted”.

For example, an “optionally substituted alkynyl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted alkynyl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “cycloalkyl” as used herein refers to an alkyl group comprising a closed ring comprising from 3 to 8 carbon atoms, for example, 3 to 6 carbon atoms. For example, a cycloalkyl group may contain 3, 4, 5, 6, 7 or 8 carbon atoms. Non-limiting examples of cycloalkyl groups include cyclopropyl, cyclobutyl, cyclopentyl, cyclohexyl, cycloheptyl, (cyclohexyl)methyl, and (cyclohexyl)ethyl. An “optionally substituted cycloalkyl” group may include the substituents as described below for the term “optionally substituted”.

For example, an “optionally substituted cycloalkyl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted cycloalkyl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “cycloalkenyl” as used herein refers to a closed non-aromatic ring comprising from 3 to 8 carbon atoms, for example, 3 to 6 carbon atoms, and which contains at least one carbon-carbon double bond. For example, a cycloalkenyl group may contain 3, 4, 5, 6, 7 or 8 carbon atoms. Non-limiting examples of cycloalkenyl groups include 1-cyclohexenyl, 4-cyclohexenyl, 1-cyclopentenyl, 2-cyclopentenyl.

An “optionally substituted cycloalkenyl” group may include the substituents as described below for the term “optionally substituted”.

For example, an “optionally substituted cycloalkenyl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted cycloalkenyl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “heterocycloalkyl” as used herein refers to a saturated or partially saturated 3 to 7 membered monocyclic, or 7 to 10 membered bicyclic ring system, which consists of carbon atoms and from one to four heteroatoms independently selected from the group consisting of O, N, and S, wherein the nitrogen and sulfur heteroatoms may be optionally oxidised, the nitrogen may be optionally quaternised, and includes any bicyclic group in which any of the above-defined rings is fused to a benzene ring, and wherein the ring may be substituted on carbon or on a nitrogen atom if the resulting compound is stable. Non-limiting examples of common saturated or partially saturated heterocycloalkyl groups include azetinyl, oxetanyl, tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups. An “optionally substituted heterocycloalkyl” group may include the substituents as described below for the term “optionally substituted”.

For example, an “optionally substituted heterocycloalkyl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted heterocycloalkyl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “alkoxy” as used herein, by itself or as part of another group, refers to an alkyl group, as defined herein, appended to the parent molecular moiety through an oxygen atom. For example, an alkoxy group may contain 1, 2, 3, 4, 5, 6, 7, 8, 9, 10, 11 or 12 carbon atoms. Preferably, the “alkoxy” as used herein, by itself or as part of another group, may refer to a straight or branched chain radical comprising from one to eight carbon atoms, more preferably one to six carbon atoms and even more preferably one to four carbon atoms, appended to the parent molecular moiety through an oxygen atom. Non-limiting examples of alkoxy groups include methoxy, ethoxy, propoxy, 2-propoxy, butoxy, tert-butoxy, pentyloxy, and hexyloxy. An “optionally substituted alkoxy” group may include the substituents as described below for the term “optionally substituted”.

For example, an “optionally substituted alkoxy” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted alkoxy” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “haloalkoxy” as used herein, by itself or as part of another group, refers to both straight and branched chain radicals of up to twelve carbon atoms, comprising at least one halogen atom and being appended to the parent molecular moiety through an oxygen atom. For example, a haloalkoxy group may contain 1, 2, 3, 4, 5, 6, 7, 8, 9, 10, 11 or 12 carbon atoms. Preferably, the term “haloalkoxy” as used herein, by itself or as part of another group, may refer to a straight or branched chain radical comprising from one to eight carbon atoms, more preferably one to six carbon atoms and even more preferably one to four carbon atoms, comprising at least one halogen atom and being appended to the parent molecular moiety through an oxygen atom.

For example, a “haloalkoxy” group may be a fluoroalkoxy or perfluoroalkoxy group.

Preferably, a “haloalkoxy” group may be a C₁-C₆ fluoroalkoxy group, or a C₁-C₆ perfluoroalkoxy group.

Even more preferably, a “haloalkoxy” group may be a C₁-C₄ fluoroalkoxy group, or a C₁-C₄ perfluoroalkoxy group. For example, a “haloalkyl” group may include difluoromethoxy, trifluoromethoxy or pentafluoroethoxy.

The term “alkanoyl” as used herein by itself or as part of another group, refers to an alkyl group, as defined herein, and appended to the parent molecular moiety through an R^{x}—C(═O)O— group via the oxygen atom, where R^{x} represents the alkyl group. For example, an alkanoyl group may contain 2, 3, 4, 5, 6, 7, 8, 9, 10, 11, 12 or 13 carbon atoms. Preferably, the term “alkanoyl” as used herein, by itself or as part of another group, may refer to a straight or branched chain radical comprising from two to eight carbon atoms, more preferably two to six carbon atoms and even more preferably two to four carbon atoms, and being appended to the parent molecular moiety through an R^{x}—C(═O)O— group via the oxygen atom, where R^{x} represents the alkyl group. Non-limiting examples of alkanoyl groups include acetoxy, propionyloxy, butyryloxy and pentanoyloxy. An “optionally substituted alkanoyl” group may include the substituents as described below for the term “optionally substituted”.

For example, an “optionally substituted alkanoyl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted alkanoyl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “amino” or “amine” as used herein refers to the group —NH₂.

The term “aryl” as used herein by itself or as part of another group refers to monocyclic, bicyclic or tricyclic aromatic groups containing from 6 to 14 carbon atoms in the ring. Common aryl groups include C₆-C₁₄ aryl, for example, C₆-C₁₀ aryl. Non-limiting examples of C₆-C₁₄ aryl groups include phenyl, naphthyl, phenanthrenyl, anthracenyl, indenyl, azulenyl, biphenyl, biphenylenyl and fluorenyl groups. An “optionally substituted aryl” group may include the substituents as described below for the term “optionally substituted”.

For example, an “optionally substituted aryl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted aryl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “heteroaryl” as used herein refers to aromatic groups having 5 to 14 ring atoms (for example, 5 to 10 ring atoms) and containing carbon atoms and 1, 2 or 3 oxygen, nitrogen or sulfur heteroatoms. Examples of heteroaryl groups include thienyl (thiophenyl), benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-α]pyrimidin-4-one, pyrazolo[1,5-α]pyrimidinyl, including without limitation pyrazolo[1,5-α]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide. An “optionally substituted heteroaryl” group may include the substituents as described below for the term “optionally substituted”.

For example, an “optionally substituted heteroaryl” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted heteroaryl” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “carbocyclic ring” as used in the context of the definition of R³ and R⁴ as defined above refers to a saturated or partially saturated divalent closed ring comprising 3 or 4 carbon atoms. Non-limiting examples of “carbocyclic rings” include cyclopropylene or cyclobutylene. An “optionally substituted carbocyclic ring” group may include the substituents as described below for the term “optionally substituted”.

For example, an “optionally substituted carbocyclic ring” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted carbocyclic ring” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

The term “heterocyclic ring” as used in the context of the definition of R³ and R⁴ as defined above refers to a saturated or partially saturated divalent closed ring comprising a 3 or 4 membered monocyclic ring system, which consists of carbon atoms and from one to three heteroatoms independently selected from the group consisting of O, N, and S, wherein the nitrogen and sulfur heteroatoms may be optionally oxidised, the nitrogen may be optionally quaternised, and wherein the ring may be substituted on carbon or on a nitrogen atom if the resulting compound is stable. Non-limiting examples of common saturated or partially saturated heterocyclic groups include aziridine-diyl, oxirane-diyl, thiirane-diyl, diazirine-diyl, azetinylene and oxetanylene groups. An “optionally substituted heterocyclic ring” group may include the substituents as described below for the term “optionally substituted”.

For example, an “optionally substituted heterocyclic ring” group may include at least one substituent selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, an “optionally substituted heterocyclic ring” group may include at least one substituent selected from halogen, hydroxy, a C₁-C₆ alkyl group, a C₁-C₆ haloalkyl group, a C₁-C₆ alkoxy group and a C₁-C₆ haloalkoxy group.

As described herein, compounds may contain “optionally substituted” moieties. In general, the term “substituted”, whether preceded by the term “optionally” or not, means that one or more hydrogen atoms of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisaged by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄ CH(OR^{o})₂; —(CH₂)₀₋₄ SR^{o}; —(CH₂)₀₋₄ Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o}C(O)NR^{o})₂; —N(R^{o})C(S)NR^{o}₂; —(CH₂)₀₋₄N(R^{o}C(O)OR^{o}; —N(R^{o})N(R^{o})C(O)R^{o}; —N(R^{o}N(R^{o}C(O)NR^{o}₂; —N(R^{o}N(R^{o}C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄ SR^{o}—; —(CH₂)₀₋₄ SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄ SSR^{o}; —(CH₂)₀₋₄ S(O)₂R^{o}; —(CH₂)₀₋₄S(O)₂OR^{o}; —(CH₂)₀₋₄₀ S(O)₂R^{o}; —S(O)₂NR^{o}₂; —(CH₂)₀₋₄ S(O)R^{o}; —N(R^{o}S(O)₂NR^{o}₂; —N(R^{o}S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o}₂; SiR^{o}₃; —(C₁₋₄ straight or branched alkylene)O—N(R^{o}₂); or —(C₁₋₄ straight or branched) alkylene)C(O)O—N(R^{o})², wherein each R^{o} may be substituted as defined below and is independently hydrogen, C₁₋₆ aliphatic, —CH₂Ph, —O(CH₂)₀₋₁ Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂ CH(OR^{•})₂, —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂ SH, —(CH₂)₀₋₂ NH₂, —(CH₂)₀₋₂ NHR^{•}, —(CH₂)₀₋₂ NR^{•}₂, —NO₂, —C(O)SR^{•}, —(C₁₋₄ straight or branched alkylene)C(O)OR^{•}, or —SSR^{•}; wherein each R^{•} is unsubstituted or where preceded by “halo” is substituted only with one or more halogens, and is independently selected from C₁₋₄ aliphatic, —CH₂Ph, —O(CH₂)₀₋₁ Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₃ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono—or bicyclic ring having 0-4 heteroatoms independently selected from nitrogen, oxygen, or sulfur.

Suitable substituents on the aliphatic group of R^{†} are independently halogen, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁ Ph, or a 5-6-membered saturated, partially unsaturated, or aryl ring having 0-4 heteroatoms independently selected from nitrogen, oxygen, or sulfur.

For example, the term “optionally substituted” as used herein may refer to when at least one substituent is selected from hydroxy, halogen, cyano, alkyl, haloalkyl, alkenyl, alkynyl, cycloalkyl, cycloalkenyl, heterocycloalkyl, alkoxy, haloalkoxy, alkanoyl, amino, aryl and heteroaryl.

Preferably, the term “optionally substituted” as used herein may refer to when at least one substituent is selected from halogen, hydroxy, a C₁-C₆ alkyl group, a C₁-C₆ haloalkyl group, a C₁-C₆ alkoxy group and a C₁-C₆ haloalkoxy group.

More preferably, the term “optionally substituted” as used herein may refer to when at least one substituent is selected from halogen, hydroxy, a C₁-C₄ alkyl group, a C₁-C₄ haloalkyl group, a C₁-C₄ alkoxy group and a C₁-C₄ haloalkoxy group.

Even more preferably, the term “optionally substituted” as used herein may refer to when at least one substituent is selected from fluoro, hydroxy, a methyl group, a trifluoromethyl group, a methoxy group and a trifluoromethoxy group.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “salt” as used herein refers to salts of the compounds as described herein that are derived from suitable inorganic and organic acids and bases. Examples of salts of an basic group include those formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁-C₄ alkyl)₄ salts. Representative alkali or alkaline earth metal salts include sodium, lithium, potassium, calcium, magnesium, and the like. Further salts include, when appropriate, nontoxic ammonium, quaternary ammonium, and amine cations formed using counterions such as halide, hydroxide, carboxylate, sulfate, phosphate, nitrate, alkyl sulfonate and aryl sulfonate.

Certain compounds of the present disclosure may exist in unsolvated forms as well as solvated forms, including hydrated forms. “Hydrate” refers to a complex formed by combination of water molecules with molecules or ions of the solute. “Solvate” refers to a complex formed by combination of solvent molecules with molecules or ions of the solute. The solvent may be an organic compound, an inorganic compound, or a mixture of both. Solvate is meant to include hydrate. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Tautomer” means compounds produced by the phenomenon wherein a proton of one atom of a molecule shifts to another atom (See, Jerry March, Advanced Organic Chemistry: Reactions, Mechanisms and Structures, Fourth Edition, John Wiley & Sons, pages 69-74 (1992)). The tautomers also refer to one of two or more structural isomers that exist in equilibrium and are readily converted from one isomeric form to another. Examples include keto-enol tautomers, such as acetone/propen-2-ol, imine-enamine tautomers and the like, ring-chain tautomers, such as glucose/2,3,4,5,6-pentahydroxy-hexanal and the like, the tautomeric forms of heteroaryl groups containing a —N═C(H)—NH— ring atom arrangement, such as pyrazoles, imidazoles, benzimidazoles, triazoles, and tetrazoles. Where the compound contains, for example, a keto or oxime group or an aromatic moiety, tautomeric isomerism (tautomerism) may occur. The compounds described herein may have one or more tautomers and therefore include various isomers. A skilled person would recognise that other tautomeric ring atom arrangements are possible. All such isomeric forms of these compounds are expressly included in the present disclosure.

“Isomers” mean compounds having identical molecular formulae but differ in the nature or sequence of bonding of their atoms or in the arrangement of their atoms in space. Isomers that differ in the arrangement of their atoms in space are termed “stereoisomers”. “Stereoisomer” and “stereoisomers” refer to compounds that exist in different stereoisomeric forms if they possess one or more asymmetric centres or a double bond with asymmetric substitution and, therefore, may be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer may be characterised by the absolute configuration of its asymmetric centre and is described by the R- and S-sequencing rules of Cahn and Prelog, or by the manner in which the molecule rotates the plane of polarised light and designated as dextrorotatory or laevorotatory (i.e., as (+) or (−)-isomers respectively). A chiral compound may exist as either individual enantiomers or as a mixture thereof. A mixture containing equal proportions of the enantiomers is called a “racemic mixture”. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 6th edition J. March, John Wiley and Sons, New York, 2007) differ in the chirality of one or more stereocentres.

The term “deuterated” as used herein alone or as part of a group, means substituted by deuterium atoms. The term “deuterated analogue” as used herein alone or as part of a group, means deuterium atoms substituted in place of hydrogen atoms. The deuterated analogue of the disclosure may be a fully or partially deuterium substituted derivative. In some embodiments, the deuterium substituted derivative of the disclosure holds a fully or partially deuterium substituted alkyl, aryl or heteroaryl group.

The disclosure also embraces isotopically-labelled compounds of the present disclosure which are identical to those recited herein, but for the fact that one or more atoms are replaced by an atom having an atomic mass or mass number different from the atomic mass or mass number usually found in nature. Examples of isotopes that may be incorporated into compounds of the disclosure include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹F, ³²F, ³⁵S, ³⁶Cl, and ¹²⁵I. Unless otherwise stated, when a position is designated specifically as “H” or “hydrogen”, the position is understood to have hydrogen at its natural abundance isotopic composition or its isotopes, such as deuterium (D) or tritium (³H). Certain isotopically-labelled compounds of the present disclosure (e.g., those labelled with ³H and ¹⁴C) are useful in compound and/or substrate tissue distribution assays. Tritiated (i.e., ³H) and carbon-14 (i.e., ¹⁴C) and fluorine-18 (i.e., ¹⁸F) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the present disclosure may generally be prepared by following procedures analogous to those described in the Schemes and in the Examples herein below, by substituting an isotopically labelled reagent for a non-isotopically labelled reagent.

### 4. Compounds

In an embodiment, a compound may have a structure according to Formula I, or a salt, a solvate, a tautomer, a stereoisomer or a deuterated analogue thereof.

In an embodiment, a compound may have a structure according to Formula IA, or a salt, a solvate, a tautomer, a stereoisomer or a deuterated analogue thereof.

In Formula I or Formula IA, X may be C═O, C═S, C═S⁺—O—, C(R³)(R⁴), C═C(R⁵)(R⁶) or C═N(R⁷), where R³ to R⁷ are as defined below.

Preferably, X may be C═O, C═S or C(R³)(R⁴).

More preferably, X may be C═O or C(R³)(R⁴).

Even more preferably, X may be C═O, CH(OH) or CH(NH₂).

Yet even more preferably, X may be C═O or CH(OH).

In Formula IA, X may be C═S.

In Formula I, in ring A, Y¹ may be C(R⁸)(R⁹), S, S(O), S(O)₂ or N(R¹⁰), Y² is C(R¹¹)(R¹²) and Y³ is C(R¹³), wherein R⁸ to R¹³ are as defined below. Preferably, Y¹ is C(R⁸)(R⁹), S or N(R¹⁰), Y² is C(R¹¹)(R¹²) and Y³ is C(R¹³). Even more preferably, Y¹ is C(R⁸)(R⁹), Y² is C(R¹¹)(R¹²) and Y³ is C(R¹³).

In Formula I, in ring A, Y¹ may be C(R⁸)(R⁹), S, S(O), S(O)₂ or N(R¹⁰), Y² is C(R¹¹) and Y³ is C, wherein R⁸ to R¹¹ are as defined below. Preferably, Y¹ is C(R⁸)(R⁹), S or N(R¹⁰), Y² is C(R¹¹) and Y³ is C. Even more preferably, Y¹ is C(R⁸)(R⁹), Y² is C(R¹¹) and Y³ is C.

In Formula I, in ring A, Y¹ may be C(R⁸) or N, Y² is C(R¹¹) and Y³ is C(R¹³), wherein R⁸, R¹¹ and R¹³ are as defined below. Preferably, Y¹ is C(R⁸), Y² is C(R¹¹) and Y³ is C(R¹³).

In Formula IA, in ring A, Y² may be C(R¹¹)(R¹²) and Y³ may be C(R¹³).

In Formula IA, in ring A, Y² may be C(R¹¹) and Y³ may be C.

In Formula I or Formula IA, R¹ may be independently selected from hydroxy, halogen, cyano, optionally substituted alkyl, haloalkyl, optionally substituted alkenyl, optionally substituted alkynyl, optionally substituted cycloalkyl, optionally substituted cycloalkenyl, optionally substituted heterocycloalkyl, optionally substituted alkoxy, haloalkoxy, optionally substituted alkanoyl, optionally substituted amino, optionally substituted aryl and optionally substituted heteroaryl.

For example, R¹ may be independently selected from hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl.

Preferably, R¹ may be independently selected from hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl.

More preferably, R¹ may be C₁-C₆ alkyl or C₁-C₆ haloalkyl, especially C₁-C₄ alkyl or C₁-C₄ haloalkyl. In particular, R¹ may be C₁-C₆ alkyl, especially C₁-C₄ alkyl.

Even more preferably, R¹ may be methyl, trifluoromethyl, ethyl, propyl or butyl. In particular, R¹ may be methyl, ethyl, propyl or butyl.

It is particularly preferred that R¹ is methyl or trifluoromethyl.

Most preferably, R¹ is methyl.

In Formula I or Formula IA, R² may be independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl.

For example, R² may be independently selected from hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl.

Preferably, R² may be independently selected from hydroxy, halogen, cyano, C₁-C₆ alkyl, C₁-C₆ haloalkyl, C₂-C₆ alkanoyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, amino, C₆-C₁₄ aryl and 5-14 membered heteroaryl.

More preferably, R² may be C₁-C₆ alkyl or C₁-C₆ haloalkyl, especially C₁-C₄ alkyl or C₁-C₄ haloalkyl. In particular, R¹ may be C₁-C₆ alkyl, especially C₁-C₄ alkyl.

Even more preferably, R² may be methyl, trifluoromethyl, ethyl, propyl or butyl. In particular, R¹ may be methyl, ethyl, propyl or butyl.

It is particularly preferred that R² is methyl or trifluoromethyl.

Most preferably, R² is methyl.

In Formula I, R³ may be independently selected from hydrogen, hydroxy, halogen, optionally substituted alkyl, optionally substituted alkoxy, optionally substituted alkanoyl and optionally substituted amino.

For example, R³ may be independently selected from hydrogen, hydroxy, halogen, optionally substituted C₁-C₆ alkyl, optionally substituted C₁-C₆ alkoxy, optionally substituted C₂-C₆ alkanoyl, and optionally substituted amino.

Preferably, R³ may be independently selected from hydrogen, hydroxy, halogen, C₁-C₆ alkyl, C₁-C₆ haloalkyl, C₁-C₆ alkoxy, C₁-C₆ haloalkoxy, C₂-C₆ alkanoyl and amino. More preferably, R³ is hydroxy.

In Formula IA, R³ may be independently selected from hydrogen, hydroxy, halogen, optionally substituted alkyl, optionally substituted alkoxy, optionally substituted alkanoyl and amino.

For example, R³ may be independently selected from hydrogen, hydroxy, halogen, optionally substituted C₁-C₆ alkyl, optionally substituted C₁-C₆ alkoxy, optionally substituted C₂-C₆ alkanoyl, and amino.

Preferably, R³ may be independently selected from hydrogen, hydroxy, halogen, C₁-C₆ alkyl, C₁-C₆ haloalkyl, C₁-C₆ alkoxy, C₁-C₆ haloalkoxy, C₂-C₆ alkanoyl and amino. More preferably, R³ is hydroxy.

In Formula I, R⁴ may be independently selected from hydrogen, hydroxy, halogen, optionally substituted alkyl, optionally substituted alkanoyl, optionally substituted alkoxy, and optionally substituted amino.

For example, R⁴ may be independently selected from hydrogen, hydroxy, halogen, optionally substituted C₁-C₆ alkyl, optionally substituted C₁-C₆ alkoxy, optionally substituted C₂-C₆ alkanoyl and optionally substituted amino.

Preferably, R⁴ may be independently selected from hydrogen, hydroxy, halogen, C₁-C₆ alkyl, C₁-C₆ haloalkyl, C₁-C₆ alkoxy, C₁-C₆ haloalkoxy, C₂-C₆ alkanoyl and amino. More preferably, R⁴ is hydrogen.

In Formula IA, R⁴ may be independently selected from hydrogen, hydroxy, halogen, optionally substituted alkyl, optionally substituted alkanoyl, optionally substituted alkoxy, and amino.

For example, R⁴ may be independently selected from hydrogen, hydroxy, halogen, optionally substituted C₁-C₆ alkyl, optionally substituted C₁-C₆ alkoxy, optionally substituted C₂-C₆ alkanoyl and amino.

Preferably, R⁴ may be independently selected from hydrogen, hydroxy, halogen, C₁-C₆ alkyl, C₁-C₆ haloalkyl, C₁-C₆ alkoxy, C₁-C₆ haloalkoxy, C₂-C₆ alkanoyl and amino. More preferably, R⁴ is hydrogen.

In Formula I or Formula IA, R³ and R⁴ may together with the carbon atom to which they are attached form a 3-membered or 4-membered optionally substituted carbocyclic or optionally substituted heterocyclic ring.

For example, R³ and R⁴ may together with the carbon atom to which they are attached form an optionally substituted cyclopropane ring, an optionally substituted cyclobutane ring, an optionally substituted aziridine ring, an optionally substituted oxirane ring, an optionally substituted thiirane ring, an optionally substituted azetidine ring, an optionally substituted oxetane ring or an optionally substituted thietane ring.

Preferably, R³ and R⁴ may together with the carbon atom to which they are attached form a cyclopropane ring, a cyclobutane ring, an aziridine ring, an oxirane ring, a thiirane ring, an azetidine ring, an oxetane ring or a thietane ring.

In Formula I or Formula IA, R⁵ may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl.

For example, R⁵ may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl.

Preferably, R⁵ may be independently selected from hydrogen, hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl.

More preferably, R⁵ may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl.

Even more preferably, R⁵ may be independently selected from hydrogen, hydroxy, fluoro, methyl, trifluoromethyl, methoxy and trifluoromethoxy.

In Formula I or Formula IA, R⁶ may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl.

For example, R⁶ may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl.

Preferably, R⁶ may be independently selected from hydrogen, hydroxy, halogen, cyano, C₁-C₆ alkyl, C₁-C₆ haloalkyl, C₂-C₆ alkanoyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, amino, C₆-C₁₄ aryl and 5-14 membered heteroaryl.

More preferably, R⁶ may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl.

Even more preferably, R⁶ may be independently selected from hydrogen, hydroxy, fluoro, methyl, trifluoromethyl, methoxy and trifluoromethoxy.

In Formula I or Formula IA, R⁷ may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl.

For example, R⁷ may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl.

Preferably, R⁷ may be independently selected from hydrogen, hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl.

More preferably, R⁷ may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl.

Even more preferably, R⁷ may be independently selected from hydrogen, hydroxy, fluoro, methyl, trifluoromethyl, methoxy and trifluoromethoxy.

In Formula I, IV may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; especially hydrogen and halogen.

For example, IV may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl; especially hydrogen and halogen.

Preferably, R⁸ may be independently selected from hydrogen, hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl; especially hydrogen and halogen.

More preferably, IV may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl; especially hydrogen and halogen.

Even more preferably, IV may be independently selected from hydrogen, hydroxy, fluoro, methyl, trifluoromethyl, methoxy and trifluoromethoxy; especially hydrogen and fluoro.

In Formula I, R⁹ may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; especially hydrogen and halogen. In particular, when R⁸ is halogen, R⁹ may also be halogen.

For example, R⁹ may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl; especially hydrogen and halogen. In particular, when R⁸ is halogen, R⁹ may also be halogen.

Preferably, R⁹ may be independently selected from hydrogen, hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl; especially hydrogen and halogen. In particular, when R⁸ is halogen, R⁹ may also be halogen.

More preferably, R⁹ may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl; especially hydrogen and halogen. In particular, when R⁸ is halogen, R⁹ may also be halogen.

Even more preferably, R⁹ may be independently selected from hydrogen, hydroxy, fluoro, methyl, trifluoromethyl, methoxy and trifluoromethoxy; especially hydrogen and fluoro. In particular, when R⁸ is fluoro, R⁹ may also be fluoro.

In Formula I, R¹⁰ may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; especially hydrogen, hydroxy and optionally substituted alkyl.

For example, R¹⁰ may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl; especially hydrogen, hydroxy and optionally substituted C₁-C₆ alkyl.

Preferably, R¹⁰ may be independently selected from hydrogen, hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl; especially hydrogen, hydroxy and C₁-C₆ alkyl.

More preferably, R¹⁰ may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl; especially hydrogen, hydroxy and C₁-C₄ alkyl.

Even more preferably, R¹⁰ may be independently selected from hydrogen, hydroxy, fluoro, methyl, trifluoromethyl, methoxy and trifluoromethoxy; especially hydrogen, hydroxy and methyl.

In Formula I or Formula IA, R¹¹ may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; especially hydrogen and halogen.

For example, R¹¹ may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl; especially hydrogen and halogen.

Preferably, R¹¹ may be independently selected from hydrogen, hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl; especially hydrogen and halogen.

More preferably, R¹¹ may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl; especially hydrogen and halogen.

Even more preferably, may be independently selected from hydrogen, hydroxy, fluoro, methyl, trifluoromethyl, methoxy and trifluoromethoxy; especially hydrogen and fluoro.

In Formula IA, R¹¹ may be halogen; especially fluoro.

In Formula I or Formula IA, R¹² may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl.

For example, R¹² may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl.

Preferably, R¹² may be independently selected from hydrogen, hydroxy, halogen, cyano, C₁-C₆ alkyl, C₁-C₆ haloalkyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, C₂-C₆ alkanoyl, amino, C₆-C₁₄ aryl and 5-14 membered heteroaryl.

More preferably, R¹² may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl.

Even more preferably, R¹² may be independently selected from hydrogen, hydroxy, fluoro, methyl, trifluoromethyl, methoxy and trifluoromethoxy.

In Formula I or Formula IA, R¹³ may be independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl.

For example, R¹³ may be independently selected from hydrogen, hydroxy, halogen, cyano, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₃-C₈ cycloalkenyl, optionally substituted 3-10 membered heterocycloalkyl, optionally substituted C₁-C₆ alkoxy, optionally substituted C₂-C₆ alkanoyl, optionally substituted amino, optionally substituted C₆-C₁₄ aryl and optionally substituted 5-14 membered heteroaryl.

Preferably, R¹³ may be independently selected from hydrogen, hydroxy, halogen, cyano, C₁-C₆ alkyl, C₁-C₆ haloalkyl, C₂-C₆ alkanoyl, C₂-C₆ alkenyl, C₂-C₆ alkynyl, C₃-C₈ cycloalkyl, C₃-C₈ cycloalkenyl, 3-10 membered heterocycloalkyl, C₁-C₆ alkoxy, C₁-C₆ haloalkoxy, amino, C₆-C₁₄ aryl and 5-14 membered heteroaryl.

More preferably, R¹³ may be independently selected from hydrogen, hydroxy, halogen, cyano, C₁-C₄ alkyl, C₁-C₄ haloalkyl, C₂-C₄ alkenyl, C₂-C₄ alkynyl, C₃-C₆ cycloalkyl, C₃-C₆ cycloalkenyl, 3-7 membered heterocycloalkyl, C₁-C₄ alkoxy, C₁-C₄ haloalkoxy, C₂-C₄ alkanoyl, amino, C₆-C₁₀ aryl and 5-10 membered heteroaryl.

Even more preferably, R¹³ may be independently selected from hydrogen, hydroxy, fluoro, methyl, trifluoromethyl, methoxy and trifluoromethoxy.

In Formula I, [symbol] represents a single or double bond to maintain correct atom valencies for Y¹, Y² and Y³ in ring A. For a given substitution pattern for Y¹, Y² and Y³, the skilled person would understand this to mean that ring A either consists of no endocyclic double bonds, or one endocyclic double bond.

For example, in the situation where Y¹ is C(R⁸)(R⁹), S, S(O), S(O)₂ or N(R¹⁰), Y² is C(R¹¹)(R¹²) and Y³ is C(R¹³), Y¹ is joined to Y² by a single bond, and Y² is joined to Y³ by a single bond.

In the situation where Y¹ is C(R⁸)(R⁹), S, S(O), S(O)₂ or N(R¹⁰), Y² is C(R¹¹) and Y³ is C, Y¹ is joined to Y² by a single bond, and Y² is joined to Y³ by a double bond.

In the situation where Y¹ is C(R⁸) or N, Y² is C(R¹¹) and Y³ is C(R¹³), Y¹ is joined to Y² by a double bond, and Y² is joined to Y³ by a single bond.

In Formula IA, [symbol] represents a single or double bond.

In a further embodiment, the compound may have a structure according to any one of Formulae I-1 to I-7, IA-1 or IA-2, wherein X and R¹ to R¹², Y¹ and Y² are as defined as above.

In a further embodiment, the compound may have a structure according to any one of Formulae II-1 to II-7, IIA-1 or IIA-2, more preferably Formulae II-1 to II-3, wherein X and R¹ to R⁷ are as defined as above.

In a further embodiment, the compound may have a structure according to any one of Formulae II-1-1 to II-1-16, more preferably Formulae II-1-1, II-1-4, II-1-5, II-1-8, II-1-9, II-1-12, II-1-13 and II-1-16, even more preferably Formulae II-1-1, II-1-5, II-1-9 and II-1-13, wherein X and R¹ to R⁷ are as defined as above.

In a further embodiment, a compound may have a structure according to any one of Formulae II-2-1 to II-2-8, more preferably Formulae II-2-1, II-2-4, II-2-5 and II-2-8, even more preferably Formulae II-2-1 and II-2-5, wherein X and R¹ to R⁷ are as defined as above.

In a further embodiment, a compound may have a structure according to any one of Formulae II-3-1 to II-3-16, more preferably Formulae II-3-1, II-3-4, II-3-5, II-3-8, II-3-9, II-3-12, II-3-13 and II-3-16, even more preferably Formulae II-3-1, II-3-5, II-3-9 and II-3-13, wherein X, R¹ to R⁷ are as defined as above.

In a further embodiment, a compound may have a structure according to any one of Formulae II-4-1 to II-4-16, more preferably Formulae II-4-1, II-4-4, II-4-5, II-4-8, II-4-9, II-4-12, II-4-13 and II-4-16, even more preferably Formulae II-4-1, II-4-5, II-4-9 and II-4-13, wherein X, R¹ to R⁷ and R¹⁰ are as defined as above.

In a further embodiment, a compound may have a structure according to any one of Formulae II-5-1 to II-5-8, more preferably II-5-1, II-5-4, II-5-5 and II-5-8, even more preferably Formulae II-5-1 and II-5-5, wherein X, R¹ to R⁷ and R¹⁰ are as defined as above.

In a further embodiment, a compound may have a structure according to any one of Formulae II-6-1 to II-6-16, more preferably Formulae II-6-1, II-6-4, II-6-5, II-6-8, II-6-9, II-6-12, II-6-13 and II-6-16, even more preferably Formulae II-6-1, II-6-5, II-6-9 and II-6-13, wherein X, R¹ to R⁷ are as defined as above.

In a further embodiment, a compound may have a structure according to any one of Formulae II-7-1 to II-7-8, more preferably Formulae II-7-1, II-7-4, II-7-5 and II-7-8, even more preferably Formulae II-7-1 and II-7-5, wherein X, R¹ to R⁷ are as defined as above.

In a further embodiment, a compound may have a structure according to any one of Formulae IIA-1-1 to IIA-1-16, preferably Formulae IIA-1-1, IIA-1-4, IIA-1-5, IIA-1-8, IIA-1-9, IIA-1-12, IIA-1-13 and IIA-1-16, even more preferably Formulae IIA-1-1, IIA-1-5, IIA-1-9 and IIA-1-13, wherein X, R¹ to R⁷ are as defined as above.

In a further embodiment, a compound may have a structure according to any one of Formulae IIA-1-1 to IIA-2-8, more preferably Formulae IIA-2-1, IIA-2-4, IIA-2-5 and IIA-2-8, even more preferably Formulae IIA-2-1 and IIA-2-5, wherein X, R¹ to R⁷ are as defined as above.

In a further embodiment, a compound may have a structure according to any one of Formulae III-1 to III-22, wherein R¹ and R² are as defined as above.

Preferably, the compound may have a structure according to any one of Formulae III-1, III-5, III-7, III-8, III-9, III-10, III-11, III-12, III-15, III-16, III-18, III-19 and III-22.

More preferably, the compound may have a structure according to any one of Formulae III-1, III-8, III-9, III-11, III-12 and III-16.

Preferably, the compound has a structure according to any one of Formulae III-1, III-2 and III-4 to III-11.

More preferably, the compound may have a structure according to any one of Formulae III-1, III-5, III-7, III-8, III-9, III-10 and III-11.

Even more preferably, the compound may have a structure according to any one of Formulae III-1, III-8, III-9 and III-11.

In a further embodiment, a compound may be selected from any one of Compounds 1-35.

Preferably, the compound is selected from any one of Compounds 1, 2, 3, 7, 8, 10, 12, 13, 16, 17, 19, 24 and 26-35.

More preferably, the compound is selected from any one of Compounds 1, 8, 12, 13, 16, 17, 19, 24, 28, 29, 31, 32 and 35.

Even more preferably, the compound is selected from any one of Compounds 1, 13, 16, 19, 24 and 29.

Preferably, the compound is selected from any one of Compounds 1-22.

More preferably, the compound is selected from any one of Compounds 1, 2, 7, 8, 10, 12, 13, 16, 17 and 19.

Even more preferably, the compound is selected from any one of Compounds Compounds 1, 8, 12, 13, 16, 17 and 19.

Yet even more preferably, the compound is selected from any one of Compounds 1, 13, 16 and 19.

Compounds as described herein may exhibit high binding affinity to olfactory proteins.

For example, the compounds may exhibit high binding affinity to olfactory binding proteins or olfactory receptors.

Preferably, the compounds exhibit high binding affinity to olfactory binding protein 6 (OBP6), and in particular, Acyrthosiphon pisum OBP6 (having an amino acid sequence as defined in SEQ ID NO: 6).

Binding affinity may be calculated using AutoDock 4.2 (Python Molecule Viewer), then screened against computer-generated models using AutoDock 4.2 and the Racoon virtual screening tool. A Lamarckian Genetic Algorithm may be used.

As used herein, a “high binding affinity” may refer to a Kᵢ value of less than 2.37 μM. In preferred embodiments, a “high binding affinity” may refer to a Kᵢ value of 2.0 μM or less, preferably 1.5 μM or less, more preferably 1.2 μM or less, even more preferably 1.0 μM or less, yet even more preferably 0.8 μM or less, most preferably 0.5 μM or less.

### 5. Compositions

In an embodiment, a composition as described herein may contain a carrier.

A carrier in a composition as described herein is any material with which the active ingredient is formulated to facilitate application to a surface, or to facilitate storage, transport or handling. A carrier may be a solid or a liquid, including a material which is normally gaseous but which has been compressed to form a liquid.

In an embodiment, the composition may be formulated for agricultural use.

An agrochemically acceptable carrier may be used.

Any of the carriers normally used in formulating agrochemical (e.g. herbicidal, fungicidal or pesticidal) compositions may be used.

Suitable solid carriers include natural and synthetic clays and silicates, for example natural silicas such as diatomaceous earths; magnesium silicates, for example talcs; magnesium aluminium silicates, for example attapulgites and vermiculites; aluminium silicates, for example kaolinites, montmorillonites and micas; calcium carbonate; calcium sulfate; ammonium sulfate; synthetic hydrated silicon oxides and synthetic calcium or aluminium silicates; elements, for example carbon and sulfur; natural and synthetic resins, for example coumarone resins, polyvinyl chloride, and styrene polymers and copolymers; solid polychlorophenols; bitumen; waxes; and solid fertilisers, for example superphosphates.

Suitable liquid carriers include water; alcohols, for example isopropanol and glycols; ketones, for example acetone, methyl ethyl ketone, methyl isobutyl ketone and cyclohexanone; ethers; aromatic or araliphatic hydrocarbons, for example benzene, toluene and xylene; petroleum fractions, for example kerosene and light mineral oils; chlorinated hydrocarbons, for example carbon tetrachloride, perchloroethylene and trichloroethane. Mixtures of different liquids are often suitable.

Agricultural compositions are often formulated and transported in a concentrated form which is subsequently diluted by the user before application. The presence of small amounts of a carrier which is a surface-active agent facilitates this process of dilution. Thus, at least one carrier in a composition as described herein may be a surface-active agent. For example, the composition may contain at least two carriers, at least one of which is a surface-active agent.

A surface-active agent may be an emulsifying agent, a dispersing agent or a wetting agent; it may be nonionic or ionic. Examples of suitable surface-active agents include the sodium or calcium salts of polyacrylic acids and lignin sulfonic acids; the condensation of fatty acids or aliphatic amines or amides containing at least 12 carbon atoms in the molecule with ethylene oxide and/or propylene oxide; fatty acid esters of glycerol, sorbitol, sucrose or pentaerythritol; condensates of these with ethylene oxide and/or propylene oxide; condensation products of fatty alcohol or alkyl phenols, for example p-octylphenol or p-octylcresol, with ethylene oxide and/or propylene oxide; sulfates or sulfonates of these condensation products; alkali or alkaline earth metal salts, preferably sodium salts, of sulfuric or sulfonic acid esters containing at least 10 carbon atoms in the molecule, for example sodium lauryl sulfate, sodium secondary alkyl sulfates, sodium salts of sulfonated castor oil, and sodium alkylaryl sulfonates such as dodecylbenzene sulfonate; and polymers of ethylene oxide and copolymers of ethylene oxide and propylene oxide.

The compositions as described herein may for example be formulated as wettable powders, dusts, granules, solutions, emulsifiable concentrates, emulsions, suspension concentrates and aerosols. Wettable powders usually contain 25, 50 or 75% w/w of active ingredient and usually contain in addition to solid inert carrier, 3-10% w/w of a dispersing agent and, where necessary, 0-10% w/w of stabiliser(s) and/or other additives such as penetrants or stickers. Dusts are usually formulated as a dust concentrate having a similar composition to that of a wettable powder but without a dispersant, and are diluted in the field with further solid carrier to give a composition usually containing 0.5-10% w/w of active ingredient. Granules are usually prepared to have a size between 10 and 100 BS mesh (1.676-0.152 mm), and may be manufactured by agglomeration or impregnation techniques. Generally, granules will contain 0.5-75% w/w active ingredient and 0-10% w/w of additives such as stabilisers, surfactants, slow release modifiers and binding agents. The so-called “dry flowable powders” consist of relatively small granules having a relatively high concentration of active ingredient. Of particular interest in current practice are the water-dispersible granular formulations. These are in the form of dry, hard granules that are essentially dust-free, and are resistant to attrition on handling, thus minimising the formation of dust. On contact with water, the granules readily disintegrate to form stable suspensions of the particles of active material. Such formulations contain 90% or more by weight of finely divided active material, 3-7% by weight of a blend of surfactants, which act as wetting, dispersing, suspending and binding agents, and 1-3% by weight of a finely divided carrier, which acts as a resuspending agent. Emulsifiable concentrates usually contain, in addition to a solvent and, when necessary, co-solvent, 10-50% w/v active ingredient, 2-20% w/v emulsifiers and 0-20% w/v of other additives such as stabilisers, penetrants and corrosion inhibitors. Suspension concentrates are usually compounded so as to obtain a stable, non-sedimenting flowable product and usually contain 10-75% w/w active ingredient, 0.5-15% w/w of dispersing agents, 0.1-10% w/w of suspending agents such as protective colloids and thixotropic agents, 0-10% w/w of other additives such as defoamers, corrosion inhibitors, stabilisers, penetrants and stickers, and water or an organic liquid in which the active ingredient is substantially insoluble; certain organic solids or inorganic salts may be present dissolved in the formulation to assist in preventing sedimentation or as anti-freeze agents for water. Aerosol recipes are usually composed of the active ingredient, solvents, furthermore auxiliaries such as emulsifiers, perfume oils, if appropriate stabilisers, and, if required, propellants.

In an embodiment, the composition may be formulated for dermatological use.

A dermatologically acceptable carrier may be used.

The carrier may provide water repellency, prevent skin irritation, and/or soothe and condition skin. Factors to consider when selecting a carrier(s) include commercial availability, cost, repellency, evaporation rate, odour, and stability. Some carriers may themselves have repellent properties.

The specific choice of a carrier, if any, to be utilised in achieving the desired intimate admixture with the final product may be any carrier conventionally used in insect repellent formulations. The carrier, moreover, may also be one that will not be harmful to the environment. Accordingly, the carrier may be any one of a variety of commercially available organic and inorganic liquid, solid, or semi-solid carriers or carrier formulations usable in formulating insect repellent products. For example, the carrier may include silicone, petrolatum, lanolin or many of several other well-known carrier components.

Examples of organic liquid carriers include liquid aliphatic hydrocarbons (e.g., pentane, hexane, heptane, nonane, decane and their analogs) and liquid aromatic hydrocarbons. Examples of other liquid hydrocarbons include oils produced by the distillation of coal and the distillation of various types and grades of petrochemical stocks, including kerosene oils which are obtained by fractional distillation of petroleum.

Other petroleum oils include those generally referred to as agricultural spray oils (e.g., the so-called light and medium spray oils, consisting of middle fractions in the distillation of petroleum and which are only slightly volatile). Such oils are usually highly refined and may contain only minute amounts of unsaturated compounds. Such oils, moreover, are generally paraffin oils and accordingly may be emulsified with water and an emulsifier, diluted to lower concentrations, and used as sprays. Tall oils, obtained from sulfate digestion of wood pulp, like the paraffin oils, may similarly be used. Other organic liquid carriers may include liquid terpene hydrocarbons and terpene alcohols such as alpha-pinene, dipentene, terpineol, and the like.

Other carriers include silicone, petrolatum, lanolin, liquid hydrocarbons, agricultural spray oils, paraffin oil, tall oils, liquid terpene hydrocarbons and terpene alcohols, aliphatic and aromatic alcohols, esters, aldehydes, ketones, mineral oil, higher alcohols, finely divided organic and inorganic solid materials.

In addition to the above-mentioned liquid hydrocarbons, the carrier may contain conventional emulsifying agents which may be used for causing the compounds to be dispersed in, and diluted with, water for end-use application.

Still other liquid carriers may include organic solvents such as aliphatic and aromatic alcohols, esters, aldehydes, and ketones. Aliphatic monohydric alcohols include methyl, ethyl, n-propyl, isopropyl, n-butyl, sec-butyl, and tert-butyl alcohols. Suitable alcohols include glycols (such as ethylene and propylene glycol) and pinacols. Suitable polyhydroxy alcohols include glycerol, arabitol, erythritol, sorbitol, and the like. Suitable cyclic alcohols include cyclopentyl and cyclohexyl alcohols.

Conventional aromatic and aliphatic esters, aldehydes and ketones may be used as carriers, and occasionally are used in combination with the above-mentioned alcohols. Still other liquid carriers include relatively high-boiling petroleum products such as mineral oil and higher alcohols (such as cetyl alcohol). Additionally, conventional or so-called “stabilisers” (e.g., tert-butyl sulfinyl dimethyl dithiocarbonate) may be used in conjunction with, or as a component of, the carrier or carriers comprising the compositions as described herein.

Solid carriers which may be used in the compositions as described herein include finely divided organic and inorganic solid materials.

Suitable finely divided solid inorganic carriers include siliceous minerals such as synthetic and natural clay, bentonite, attapulgite, fuller's earth, diatomaceous earth, kaolin, mica, talc, finely divided quartz, and the like, as well as synthetically prepared siliceous materials, such as silica aerogels and precipitated and fume silicas. Examples of finely divided solid organic materials include cellulose, sawdust, synthetic organic polymers, and the like. Examples of semi-solid or colloidal carriers include waxy solids, gels (such as petroleum jelly), lanolin, and the like, and mixtures of well-known liquid and solid substances which may provide semi-solid carrier products, for providing effective repellency.

Compositions as described herein may also contain adjuvants known in the art of personal care product formulations, such as thickeners, buffering agents, chelating agents, preservatives, fragrances, antioxidants, gelling agents, stabilisers, surfactants, emolients, coloring agents, aloe vera, waxes, other penetration enhancers and mixtures thereof, and therapeutical or cosmetically active agents.

Additionally, the compositions as described herein may also contain other adjuvants such as one or more therapeutically or cosmetically active ingredients. Exemplary therapeutic or cosmetically active ingredients useful in the compositions as described herein include fungicides, sunscreening agents, sunblocking agents, vitamins, tanning agents, plant extracts, anti-inflammatory agents, anti-oxidants, radical scavenging agents, retinoids, alpha-hydroxy acids, emollients, antiseptics, antibiotics, antibacterial agents or antihistamines, and may be present in an amount effective for achieving the therapeutic or cosmetic result desired.

The compositions as described herein may be formulated and packaged so as to deliver the product in a variety of forms including as a solution, suspension, cream, ointment, gel, film or spray, depending on the preferred method of use. The carrier may be an aerosol composition adapted to disperse the compounds into the atmosphere by means of a compressed gas.

In an embodiment, the compositions as described herein may comprise at least one additional active ingredient.

For example, the additional active ingredient may be an insecticide.

Non-limiting examples of insecticides may include aldrin, chlordane, chlordecone, DDT, dieldrin, endosulfan, endrin, heptachlor, hexachlorobenzene, lindane, methoxychlor, mirex, pentachlorophenol, dichlorodiphenyldichloroethane, acephate, azinphos-methyl, bensulide, chlorethoxyfos, chlorpyrifos, chlorpyrifos-methyl, diazinon, dichlorvos, dicrotophos, dimethoate, disulfoton, ethoprop, fenamiphos, fenitrothion, fenthion, fosthiazate, malathion, methamidophos, methidathion, mevinphos, monocrotophos, naled, omethoate, oxydemeton-methyl, parathion, parathion-methyl, phorate, phosalone, phosmet, phostebupirim, phoxim, pirimiphos-methyl, profenofos, terbufos, tetrachlorvinphos, tribufos, trichlorfon, aldicarb, bendiocarb, carbofuran, carbaryl, dioxacarb, fenobucarb, fenoxycarb, isoprocarb, methomyl, oxamyl, propoxur, 2-(1-methylpropyl)phenyl methylcarbamate, allethrin, bifenthrin, cyhalothrin, cypermethrin, cyfluthrin, deltamethrin, etofenprox, fenvalerate, permethrin, phenothrin, prallethrin, resmethrin, tetramethrin, tralomethrin, transfluthrin, acetamiprid, clothianidin, dinotefuran, imidacloprid, nithiazine, thiacloprid, thiamethoxam, chlorantraniliprole, cyantraniliprole, flubendiamide, diflubenzuron, flufenoxuron, cyromazine, methoprene, hydroprene, tebufenozide, anabasine, anethole, annonins, pawpaw tree seeds, azadirachtin, caffeine, Carapa, cinnamaldehyde, cinnamon leaf oil, cinnamyl acetate, citral, deguelin, Derris, Desmodium caudatum, eugenol, ivermectin, linalool, myristicin, Neem oil, nicotine, Peganum harmala, oregano oil, Quassia, ryanodine, rotenone, spinosad, spinosyn A, spinosyn D, tetranortriterpenoid, thymol, Bacillus sphaericus, Bacillus thuringiensis, Bacillus thuringiensis aizawi, Bacillus thuringiensis israelensis, Bacillus thuringiensis kurstaki, Bacillus thuringiensis tenebrionis, nuclear polyhedrosis virus, granulovirus, Lecanicillium lecanii, diatomaceous earth, borax and boric acid.

For example, the additional active ingredient may be an insect repellent.

Non-limiting examples of insect repellents may include methyl anthranilate, benzaldehyde, N,N-diethyl-m-toluamide, dimethyl carbate, dimethyl phthalate, ethylhexanediol, icaridin, butopyronoxyl, ethyl butylacetylaminopropionate, metofluthrin, SS220, tricyclodecenyl allyl ether, VUAA1, Callicarpa, birch tree bark, Myrica gale, catnip oil, citronella oil, eucalyptus oil, lemon eucalyptus essential oil, p-menthane-3,8-diol, Neem oil, nepetalactone, nepetalactol, lemongrass, tea tree oil, tobacco, Achillea alpine, alpha-terpinene, basil, sweet basil, breadfruit, camphor, carvacrol, castor oil, cedar oil, celery extract, cinnamon, oil of cloves, fennel oil, garlic, geranium oil, lavender, marigold, marjoram, mint, menthol, oleic acid, Mentha pulegium, peppermint, rosemary, Lantana camara, thyme, yellow nightshade and Andrographis paniculata.

### 6. Uses and Methods for Modulating Insect Behaviour

Compounds and compositions as described herein may be used to modulate insect behaviour.

In an embodiment, the compounds and compositions as described herein may be used as an insect repellent.

In another embodiment, the compounds and compositions as described herein may be used as an insect attractant.

In another embodiment, the compounds and compositions as described herein may be used as an insect mating disruptant.

As used herein, an “insect repellent” is a substance applied to surfaces which discourages insects from landing on or coming into close proximity to that surface. For example, an insect repellent may include substances that are noxious to the insect. For example, an insect repellent may include an alarm pheromone.

As used herein, an “insect attractant” is a substance applied to surfaces which encourages insects to land on or come into close proximity to that surface. For example, an insect attractant may include substances such as naturally derived or synthetic pheromones, especially sex pheromones.

As used herein, an “insect mating disruptant” is a substance that interferes with the normal mating behaviour of insects, thereby affecting the chance of reproduction. For example, an insect mating disruptant may confuse male insects and limit their ability to locate calling females.

In an embodiment, the insect may be selected from aphids, lacewings, houseflies, mosquitoes, cockroaches, mites and ticks.

Preferably, the insect is an aphid.

More preferably, the insect may be selected from a pea aphid, black bean aphid, soybean aphid, Spiraea aphid/green citrus aphid, leaf-curling plum aphid, mealy cabbage aphid, rosy apple aphid, mealy plum aphid, potato aphid, peach-potato aphid, damson-hop aphid, bird cherry-oat aphid, green bug aphid, grain aphid, blackberry-grass aphid, tea aphid and peach aphid.

Even more preferably, the insect is the pea aphid, Acyrthosiphon pisum.

In an embodiment, a method of modulating insect behaviour is provided.

The method may comprise application of the compounds or compositions as described herein to a locus.

As used herein, the term “locus” broadly encompasses the fields on which the treated plants are growing, or where the seeds of cultivated plants are sown, or the place where the seed will be placed into the soil.

In an embodiment, the locus may be a plant or a part thereof.

For example, compounds and compositions described herein can be administered to seeds or plants wherein the control of insect behaviour is desired.

As used herein, the term “seed” broadly encompasses plant propagating material such as, tubers cuttings, seedlings, seeds, and germinated or soaked seeds.

The compounds and compositions described herein can be administered to the environment of plants (e.g., soil) wherein the control of insect behaviour is desired.

A compound or composition as described herein may act as an insect repellent or insect mating disruptant to keep insects (for example, pests) that may be detrimental to the health of the plant away.

A compound or composition as described herein may act as an insect attractant to attract other insects to a locus thereof, for example, the plant or parts thereof. These other insects may act as a biological control for insects (for example, pests) that may be detrimental to the health of the plant.

Non-limiting examples of plants to which the control of insect behaviour may be applied, in accordance with the methods described herein, include monocotyledonous crops such as corn, wheat, barley, rye, rice, sorghum, oat; sugarcane and turf; and dicotyledonous crops such as cotton, sugar beet, peanut, potato, sweet potato, yam, sunflower, soybean, alfalfa, canola, grapes, tobacco; vegetables including Solanaceae vegetables such as eggplant, tomato, green pepper and pepper; Cucurbitaceae vegetables such as cucumber, pumpkin, zucchini, watermelon, melon and squash; Brassicaceae vegetables such as radish, turnip, horseradish, Chinese cabbage, cabbage, leaf mustard, broccoli and cauliflower; Asteraceae vegetables such as artichoke and lettuce; Liliaceae vegetables such as leek, onion, garlic and asparagus; Apiaceae vegetables such as carrot, parsley, celery and parsnip; Chenopodiaceae vegetables such as spinach and chard; Lamiaceae vegetables such as mint and basil; flowers such as petunia, morning glory, carnation, chrysanthemum and rose; foliage plants; fruit trees such as pome fruits (e.g., apple, pear and Japanese pear), stone fruits (e.g., peach, plum, nectarine, cherry, apricot and prune), citrus (e.g., orange, lemon, lime and grapefruit), tree nuts (e.g., chestnut, pecan, walnut, hazel, almond, pistachio, cashew and macadamia), berries such as blueberry, cranberry, blackberry, strawberry and raspberry; persimmon; olive; loquat; banana; coffee; palm; coco; the other trees such tea, mulberry, flower trees, and landscape trees (e.g., ash, birch, dogwood, eucalyptus, ginkgo, lilac, maple, oak, poplar, Formosa sweetgum, sycamore, fir, hemlock fir, needle juniper, pine, spruce, yew).

Preferably, the plant to which the control of insect behaviour may be applied is selected from grasses, cereal crops such as wheat, maize, and barley, oilseeds such as rapeseed, sugar beet, cabbages, beans, cotton, sugarcane, cassava, pulses, peas, tea, vegetables, potatoes, brassicas, cowpeas, citrus fruits, apples, plums, damsons, peaches, grapes, soft fruit, lettuce, blackcurrants, berries, chestnuts, alfalfa, clover, beans, peas, chickpeas, lentils, lupins, mesquite, carob, soybeans, peanuts and tamarind.

More preferably, the plant to which the control of insect behaviour may be applied is selected from peas and field beans.

Even more preferably, the insect and plant to which the control of insect behaviour may be applied is selected from at least one of the following:

| Insect | Species Name | Plant |
|---|---|---|
| Pea aphid | Acyrthosiphon pisum | Peas, field beans |
| Black bean aphid | Aphis fabae | Field beans, sugar |
| | | beet |
| Soybean aphid | Aphis glycines | Soybean |
| Spiraea aphid/Green | Aphis spiraecola | Citrus crops |
| citrus aphid | | |
| Leaf-curling plum | Brachycaudus helichrysi | Plum, damson |
| aphid | | trees |
| Mealy cabbage aphid | Brevicoryne brassicae | Oilseeds, |
| | | vegetable brassicas |
| Rosy apple aphid | Dysaphis plantaginea | Apple trees |
| Mealy plum aphid | Hyalopterus pruni | Plum tree |
| Potato aphid | Macrosiphum euphorbiae | Brassicas, |
| | | potatoes, sugar |
| | | beet, lettuce |
| Peach-potato aphid | Myzus persicae | Oilseeds, |
| | | brassicas, sugar |
| | | beet, lettuce |
| Damson-hop aphid | Phorodon humuli | Prunus Spp. |
| Bird cherry-oat aphid | Rhopalosiphum padi | Cereal crops |
| Green bug aphid | Schizaphis graminum | Cereal |
| | | crops/grasses |
| Grain aphid | Sitobion avenae | Cereal crops, |
| | | potatoes |
| Blackberry-grass | Sitobion fragariae | Grasses |
| aphid | | |
| Tea aphid | Toxoptera aurantii | Many plant spp. |
| Peach aphid | Tuberocephalus momonis | Peach |

Generally, the methods described herein can be used to modulate insect behaviour on various parts of agricultural crop plants (e.g., fruit, blossoms, leaves, stems, tubers, roots) or other useful plants as described herein.

For example, methods described herein may be used to modulate insect behaviour with regard to vegetable crops, row crops, trees, nuts, vines, turf, and ornamental plants.

The methods described herein may also be used to modulate insect behaviour in horticulture. For example, methods described herein may be used to modulate insect behaviour on roses.

A compound or composition as described herein may be supplied to a plant exogenously. The compound or composition may be applied to the plant and/or the surrounding soil through sprays, drips, and/or other forms of liquid application.

The compounds described herein may penetrate the plant through the roots via the soil (systemic action); by drenching the locus of the plant with a liquid composition; or by applying the compounds in solid form to the soil, e.g. in granular form (soil application).

A compound or composition as described herein may be applied to a plant, including plant leaves, shoots, roots, or seeds. For example, compound or composition as described herein can be applied to a foliar surface of a plant. Foliar applications may require 50 to 500 g per hectare of a compound as described herein.

As used herein, the term “foliar surface” broadly refers to any green portion of a plant having surface that may permit absorption of silicon, including petioles, stipules, stems, bracts, flowerbuds, and leaves. Absorption commonly occurs at the site of application on a foliar surface, but in some cases, the applied compound or composition may run down to other areas and be absorbed there.

Compounds or compositions described herein can be applied to the foliar surfaces of the plant using any conventional system for applying liquids to a foliar surface. For example, application by spraying will be found most convenient. Any conventional atomisation method can be used to generate spray droplets, including hydraulic nozzles and rotating disk atomisers. In other instances, alternative application techniques, including application by brush or by rope-wick, may be utilised.

A compound or composition as described herein can be directly applied to the soil surrounding the root zone of a plant. Soil applications may require 0.5 to 5 kg per hectare of a compound as described herein on a broadcast basis (rate per treated area if broadcast or banded).

For example, a compound or composition as described herein may be applied directly to the base of the plants or to the soil immediately adjacent to the plants.

In some embodiments, a sufficient quantity of the compound or composition is applied such that it drains through the soil to the root area of the plants.

Generally, application of a compound or composition as described herein may be performed using any method or apparatus known in the art, including but not limited to hand sprayer, mechanical sprinkler, or irrigation, including drip irrigation.

A compound or composition as provided herein can be applied to plants and/or soil using a drip irrigation technique. For example, the compound or composition may be applied through existing drip irrigation systems. For example, this procedure can be used in connection with cotton, strawberries, tomatoes, potatoes, vegetables, and ornamental plants.

In other embodiments, a compound or composition as described herein can be applied to plants and/or soil using a drench application. For example, the drench application technique may be used in connection with crop plants and turf grasses.

A compound or composition as described herein may be applied to soil after planting. Alternatively, a composition as described herein may be applied to soil during planting, or may be applied to soil before planting.

For example, a compound or composition as described herein may be tilled into the soil or applied in furrow.

In crops grown in water, such as rice, solid granulates comprising the compounds described herein may be applied to the flooded field or locus of the crop plants to be treated.

For example, the method may comprise treating a seed with a compound or composition as described herein.

For example, a compound as described herein may be applied to seeds or tubers by impregnating them with a liquid seed treatment composition comprising a compound described herein, or by coating them with a solid or liquid composition comprising a compound described herein.

Seed treatment methods described herein can be used in connection with any species of plant and/or the seeds thereof as described herein. Typically, the methods are used in connection with seeds of plant species that are agronomically important. In particular, the seeds can be of corn, peanut, canola/rapeseed, soybean, cucurbits, crucifers, cotton, beets, rice, sorghum, sugar beet, wheat, barley, rye, sunflower, tomato, sugarcane, tobacco, oats, as well as other vegetable and leaf crops. For example, the seed can be corn, soybean, or cotton seed. The seed may be a transgenic seed from which a transgenic plant can grow and incorporate a transgenic event that confers, for example, tolerance to a particular herbicide or combination of herbicides, insect resistance, increased disease resistance, enhanced tolerance to stress and/or enhanced yield. Transgenic seeds include, but are not limited to, seeds of corn, soybean and cotton.

A seed treatment method may comprise applying the seed treatment composition to the seed prior to sowing the seed, so that the sowing operation is simplified. In this manner, seeds can be treated, for example, at a central location and then dispersed for planting. This permits the person who plants the seeds to avoid the complexity and effort associated with handling and applying the compositions, and to merely handle and plant the treated seeds in a manner that is conventional for regular untreated seeds.

A composition as described herein can be applied to seeds by any standard seed treatment methodology, including but not limited to mixing in a container (e.g., a bottle or bag), mechanical application, tumbling, spraying, immersion, and solid matrix priming. Seed coating methods and apparatus for their application are disclosed in, for example, U.S. Pat. Nos. 5,918,413; 5,891,246; 5,554,445; 5,389,399; 5,107,787; 5,080,925; 4,759,945 and 4,465,017, among others. Any conventional active or inert material can be used for contacting seeds with the composition, such as conventional film-coating materials including but not limited to water-based film coating materials.

For example, a composition as described herein can be introduced onto or into a seed by use of solid matrix priming. For example, a quantity of the composition can be mixed with a solid matrix material and then the seed can be placed into contact with the solid matrix material for a period to allow the composition to be introduced to the seed. The seed can then optionally be separated from the solid matrix material and stored or used, or the mixture of solid matrix material plus seed can be stored or planted directly. Non-limiting examples of solid matrix materials which are useful include polyacrylamide, starch, clay, silica, alumina, soil, sand, polyurea, polyacrylate, or any other material capable of absorbing or adsorbing the composition for a time and releasing the active compound of the composition into or onto the seed. It is useful to make sure that the active compound and the solid matrix material are compatible with each other. For example, the solid matrix material may be chosen so that it can release the active compound at a reasonable rate, for example over a period of minutes, hours, days, or weeks.

Imbibition is another method of treating seed with the composition. For example, a plant seed can be directly immersed for a period of time in the composition. During the period that the seed is immersed, the seed takes up, or imbibes, a portion of the composition. Optionally, the mixture of plant seed and the composition can be agitated, for example by shaking, rolling, tumbling, or other means. After imbibition, the seed can be separated from the composition and optionally dried, for example by patting or air drying.

A composition as described herein may be applied to the seeds using conventional coating techniques and machines, such as fluidised bed techniques, the roller mill method, rotostatic seed treaters, and drum coaters. Other methods, such as spouted beds may also be useful. The seeds may be pre-sized before coating. After coating, the seeds may be dried and then transferred to a sizing machine for sizing. Such procedures are generally known in the art.

If a composition as described herein is applied to the seed in the form of a coating, the seeds can be coated using a variety of methods known in the art. For example, the coating process can comprise spraying the composition onto the seed while agitating the seed in an appropriate piece of equipment such as a tumbler or a pan granulator.

When coating seed on a large scale (for example a commercial scale), the seed coating may be applied using a continuous process. For example, seed may be introduced into the treatment equipment (such as a tumbler, a mixer, or a pan granulator) either by weight or by flow rate. The amount of treatment composition that is introduced into the treatment equipment can vary depending on the seed weight to be coated, surface area of the seed, the concentration of the compound and/or other active ingredients in a composition, the desired concentration on the finished seed, and the like. A composition as described herein can be applied to the seed by a variety of means, for example by a spray nozzle or revolving disc. The amount of liquid may be determined by the assay of the formulation and the required rate of active ingredient necessary for efficacy. As the seed falls into the treatment equipment the seed can be treated (for example by misting or spraying with the composition) and passed through the treater under continual movement/tumbling where it can be coated evenly and dried before storage or use.

The seed coating may be applied using a batch process. For example, a known weight of seeds can be introduced into the treatment equipment (such as a tumbler, a mixer, or a pan granulator). A known volume of the composition can be introduced into the treatment equipment at a rate that allows the composition to be applied evenly over the seeds. During the application, the seed can be mixed, for example by spinning or tumbling. The seed can optionally be dried or partially dried during the tumbling operation. After complete coating, the treated sample can be removed to an area for further drying or additional processing, use, or storage.

The seed coating may be applied using a semi-batch process that incorporates features from each of the batch processes and continuous processes set forth above.

In other embodiments, seeds can be coated in laboratory size commercial treatment equipment such as a tumbler, a mixer, or a pan granulator by introducing a known weight of seeds in the treater, adding the desired amount of the composition, tumbling or spinning the seed and placing it on a tray to thoroughly dry.

Seeds can also be coated by placing the known amount of seed into a narrow neck bottle or receptacle with a lid. While tumbling, the desired amount of the composition can be added to the receptacle. The seed is tumbled until it is coated with the composition. After coating, the seed can optionally be dried, for example on a tray.

In an embodiment, the locus may be an insect trap.

A compound or composition as described herein may act as an insect attractant to lure the insects to the trap.

For example, the insect trap may be configured to trap and/or kill an array of insects, for example, aphids, lacewings, houseflies, mosquitoes, cockroaches, mites and ticks. The insect trap may be arranged to actively kill the insects, for example by electrocution or by chemical means, and/or to passively kill the insects, for example by starvation.

The insect trap may hold the insects for a length of time (e.g. for at least one hour to a day). This may be advantageous for monitoring a particular insect population.

The insect trap may comprise additional lure for attracting insects. The additional lure may be a light source, for example in the UV spectrum. The additional lure may be an LED lamp and/or a fluorescent lamp. The additional lure may be a heat source. The additional lure may be some bait, a gas producing element (for example, a CO₂ producing element) and/or a scent producing element.

The insect trap may comprise a means for retaining insects within the insect trap. For example, the insect trap may comprise glue boards and/or an insect trapping volume from which insects struggle to escape.

The insect trap may comprise a means for retaining insect carcasses. For example, the insect trap may comprise glue boards and/or an insect carcass collection area, for example a tray.

The insect trap may comprise a means to actively kill the insects. For example, the insect trap may comprise a conducting element for electrocuting insects, gas to asphyxiate insects and/or poison to poison the insects.

### 7. Olfactory Proteins

Olfactory proteins are responsible for the detection of odorants and may therefore be suitable for the detection of sex pheromones associated with insects, for example aphids.

Olfactory proteins may include odorant receptors (ORs). Non-limiting examples of odorant receptors include odorant receptor 1/olfactory receptor co-receptor (ORVORCO), odorant receptor 2 (OR2), odorant receptor 4 (OR4), odorant receptor 5 (OR5), odorant receptor 10 (OR10), odorant receptor 17 (OR17), odorant receptor 20 (OR20), odorant receptor 22c (OR22c), odorant receptor 23 (OR23), odorant receptor 25 (OR25), odorant receptor 31 (OR31), odorant receptor 37 (OR37), odorant receptor 38 (OR38), odorant receptor 39 (OR39), odorant receptor 42 (OR42) and odorant receptor 43 (OR43).

Olfactory proteins may include olfactory binding proteins (OBPs). Non-limiting examples of olfactory binding proteins include olfactory binding protein 1 (OBP1), olfactory binding protein 2 (OBP2), olfactory binding protein 3 (OBP3), olfactory binding protein 4 (OBP4), olfactory binding protein 5 (OBP5), olfactory binding protein 6 (OBP6), olfactory binding protein 7 (OBP7), olfactory binding protein 8 (OBP8), olfactory binding protein 9 (OBP9), olfactory binding protein 10 (OBP10) and olfactory binding protein 11 (OBP11).

### 8. Biosensors

Olfactory proteins described herein, or variants or fragments thereof may be used in a biosensor.

The biosensor may be suitable for detecting an analyte in a sample.

For example, a biosensor may comprise olfactory proteins from aphids.

For example, a biosensor may comprise olfactory proteins from Acyrthosiphon pisum.

For example, a biosensor may comprise olfactory binding proteins, or variants or fragments thereof.

For example, a biosensor may comprise OBP6, or a variant or fragment thereof.

For example, a biosensor may comprise Acyrthosiphon pisum OBP6 (having an amino acid sequence as defined in SEQ ID NO: 6), or a variant or a fragment thereof.

The term “variant” or “functional variant” as used herein with reference to any of the sequences described herein refers to a variant polypeptide sequence or part of the polypeptide sequence which retains the biological function of the full non-variant sequence. A functional variant also comprises a variant of the polypeptide of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a polypeptide sequence that does not affect the functional properties of the polypeptide are well known in the art. For example, the amino acid alanine, a hydrophobic amino acid, may be substituted by another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

As used in any aspect described herein, a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant amino acid sequence.

Preferably, a “variant” or a “functional variant” as described herein has at least 60%, 70%, 75%, 80%, 85%, 90%, 95% or 99% overall sequence identity to the non-variant amino acid sequence.

More preferably, a “variant” or a “functional variant” as described herein has at least 70%, 75%, 80%, 85%, 90%, 95% or 99% overall sequence identity to the non-variant amino acid sequence.

Even more preferably, a “variant” or a “functional variant” as described herein has at least 80%, 85%, 90%, 95% or 99% overall sequence identity to the non-variant amino acid sequence.

It is particularly preferred that a “variant” or a “functional variant” as described herein has at least 90%, 95% or 99% overall sequence identity to the non-variant amino acid sequence.

Two polypeptides are said to be “identical” if the sequence of amino acid residues, respectively, in the two sequences is the same when aligned for maximum correspondence as described below. The terms “identical” or percent “identity”, in the context of two or more polypeptide sequences, refer to two or more sequences or subsequences that are the same or have a specified percentage of amino acid residues that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

The term “fragment”, as used herein, refers to a functionally active series of consecutive amino acids from a longer polypeptide or protein. For example, the fragment may have a high binding affinity to the sex pheromones of insects. Preferably, the fragment has a high binding affinity to the sex pheromones of aphids. Even more preferably, the fragment has a high binding affinity to the sex pheromones of Acyrthosiphon pisum. It is particularly preferred that the fragment has a high binding affinity to nepetalactols or nepetalactones, in particular, (1R,4aS,7S,7aR)-nepetalactol ((1R,4aS,7S,7aR)-4,7-dimethyl-1,4a,5,6,7,7a-hexahydrocyclopenta[c]pyran-1-ol), (4aS,7S,7aR)-nepetalactone ((4aS,7S,7aR)-4,7-dimethyl-5,6,7,7a-tetrahydrocyclopenta[c]pyran-1(4aH)-one), (1S,4aR,7R,7aS)-nepetalactol ((1S,4aR,7R,7aS)-4,7-dimethyl-1,4a,5,6,7,7a-hexahydrocyclopenta[c]pyran-1-ol) and/or (4aR,7R,7aS)-nepetalactone 44aR,7R,7aS)-4,7-dimethyl-5,6,7,7a-tetrahydrocyclopenta[c]pyran-1(4aH)-one).

In an embodiment, the biosensor may further comprise a signal generator. For example, the signal generator may be a transducer.

The signal generator may be configured to output a signal when the analyte has bound to the olfactory protein.

The signal generator may be configured to output a different signal when the analyte is not bound to the olfactory protein.

The signal generator may be configured to output no signal when the analyte is not bound to the olfactory protein.

A skilled person would also understand that a situation where the analyte has not bound to the olfactory protein leads to an output of a signal, and where the analyte has bound to the olfactory protein leads to an output of no signal, would be embraced by the present disclosure.

A magnitude of the signal generated may be correlated or proportional to the concentration of analyte in the sample. For example, a higher concentration of analyte may be associated with a more intense signal in terms of magnitude; conversely, a lower concentration of analyte may be associated with a less intense signal in terms of magnitude.

The signal may be based on an electrical signal. For example, the signal may be a potential difference, a current, a resistance, a capacitance or an impedance associated with the electrical signal.

The signal may be based on an optical signal. For example, the signal may be based on various properties of electromagnetic radiation, such as wavelength, intensity, absorption, scattering or fluorescence. For example, the electromagnetic radiation may be ultraviolet light, visible light or infrared light.

The biosensor may operate based on electrochemical means. Binding of an analyte may cause consumption or generation of electrons. Alternatively, binding of an analyte does not cause direct electron flow but causes changes on an electrode surface, for example, changes in charge, hydration or pH.

Non-limiting examples of an electrochemical-based biosensor include impedimetric, amperometric, potentiometric, conductometric and voltametric biosensors. An electrochemical-based biosensor may include biosensor field-effect transistors (BioFETs), such as ion-sensitive field-effect transistors (ISFETs).

The biosensor may operate based on optical means. For example, the biosensor may operate in a label-free mode, where an optical signal is generated directly by the interaction of the analyte with a transduction element. Alternatively, the biosensor may operate with a label and an optical signal is generated by a colorimetric, fluorescent or luminescent method.

Non-limiting examples of an optical-based biosensor include surface plasmon resonance (SPR), evanescent wave fluorescence, optical waveguide interferometric, ellipsometric, reflectometric interference spectroscopy (RIfS) and surface-enhanced Raman scattering (SERS) biosensors.

The biosensor may operate based on piezoelectric means. For example, the biosensor may include a piezoelectric crystal (e.g. quartz) which vibrates under the influence of an electric field. The resonant frequency may change as an analyte adsorbs or desorbs from the surface of the piezoelectric crystal.

In an embodiment, the biosensor may further comprise a flow path for moving a sample, wherein the sample may contain an analyte.

In an embodiment, the biosensor may further comprise a substrate.

For example, the substrate may be a glass substrate or a polymer substrate. The substrate may exhibit excellent mechanical strength, thermal stability, transparency, surface smoothness, ease of handling, and water resistance.

In an embodiment, the biosensor may further comprise a protein-containing layer immobilised to the substrate, wherein the protein-containing layer contains the olfactory protein. The protein-containing layer may be in contact with the flow path.

Means for immobilising proteins onto a substrate are well known in the art. Non-limiting methods of immobilising proteins include adsorption, cross-linking, covalent bonding, electrochemical polymerisation and photopolymerisation.

In an embodiment, the biosensor may further comprise a working electrode.

In an embodiment, the biosensor may further comprise a counter electrode.

The working electrode and/or the counter electrode may be partially exposed at the flow path and utilised for applying a voltage to a sample.

The working electrode and/or the counter electrode may comprise an electrically conductive material. An electrically conductive material is any material that allows the flow of charge (electrical current) in one or more directions. Non-limiting examples of electrically conductive materials may include metals and metal alloys, such as gold, platinum, copper, silver, aluminium, palladium, steel, brass and bronze; carbon-based materials, such as graphite, single-wall carbon nanotubes (SWCNTs), multi-wall carbon nanotubes (MWCNTs), graphene, graphene oxide; metal oxides, such as indium tin oxide (ITO), indium zinc oxide (IZO), tin oxide, zinc oxide; and organic conducting polymers, such as polythiophene, polyaniline and polypyrrole.

In an embodiment, the biosensor may further comprise a reference electrode.

The reference electrode may provide a reference signal, for example, a stable potential, relative to the sample. For example, a reference electrode may be an Ag/AgCl electrode.

Biosensors as described herein may have high binding affinity to the sex pheromones of insects and therefore may be highly sensitive to the sex pheromones of insects.

For example, biosensors as described herein may be highly sensitive to the sex pheromones of aphids, and in particular, Acyrthosiphon pisum.

Biosensors as described herein may be highly sensitive to the presence of nepetalactols or nepetalactones.

For example, biosensors as described herein may be highly sensitive to the presence of (1R,4aS,7S,7aR)-nepetalactol ((1R,4aS,7S,7aR)-4,7-dimethyl-1,4a,5,6,7,7a-hexahydrocyclopenta[c]pyran-1-ol), (4aS,7S,7aR)-nepetalactone ((4aS,7S,7aR)-4,7-dimethyl-5,6,7,7a-tetrahydrocyclopenta[c]pyran-1(4aH)-one), (1S,4aR,7R,7aS)-nepetalactol ((1S,4aR,7R,7aS)-4,7-dimethyl-1,4a,5,6,7,7a-hexahydrocyclopenta[c]pyran-1-ol) and/or (4aR,7R,7aS)-nepetalactone ((4aR,7R,7aS)-4,7-dimethyl-5,6,7,7a-tetrahydrocyclopenta[c]pyran-1(4aH)-one).

In particular, the biosensors as described herein may be highly sensitive to the presence of natural nepetalactols or nepetalactones, such as (1R,4aS,7S,7aR)-nepetalactol and (4aS,7S,7aR)-nepetalactone.

As used herein, the term “highly sensitive” may refer to a biosensor comprising an olfactory protein as described herein, or a fragment or variant thereof, having a binding energy of −7.0 kcal mol⁻¹, preferably −7.1 kcal mol⁻¹, more preferably −7.2 kcal mol⁻¹, even more preferably −7.3 kcal mol⁻¹, yet even more preferably −7.4 kcal mol⁻¹, most preferably −7.5 kcal mol⁻¹, to a nepetalactol or nepetalactone as described herein.

### 9. Uses of Biosensors and Methods for Detecting Analytes

Biosensors as described herein may generally be used for the detection of analytes.

In an embodiment, the biosensors described herein may be used to identify new olfactory ligands.

For example, the new olfactory ligands may be associated with aphids, and in particular, Acyrthosiphon pisum.

For example, the biosensor may be used as a lab screening tool to identify new olfactory ligands. For example, the biosensor may be used to identify aphid attractants other than sex pheromone components.

In an embodiment, the biosensors described herein may be used in high-throughput screening.

High-throughput screening (HTS) typically uses automated assays to search through large numbers of compounds for a desired activity. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

In an embodiment, the biosensors described herein may be used to detect field populations of aphids.

For example, the biosensor may be deployed as a field tool.

In an embodiment, a method of detecting an analyte in a sample is provided, comprising: (a) providing a biosensor as described herein; (b) contacting the biosensor with the sample; and (c) comparing a magnitude of the signal generated by the biosensor when the sample is present with a reference magnitude of the signal generated by the biosensor when the sample is absent.

### EXPERIMENTAL

The following non-limiting examples are provided for further illustration.

### Examples 1 to 22: Ligand-Protein Modelling and Docking

Sequence Data and Protein Models

All previously published protein structures were accessed from the Protein Data Bank (PDB).

TABLE 1
NCBI accession codes for Acyrthosiphon pisum olfactory proteins.

| A. Pisum Protein | Residues | NCBI Ascension Code |
|---|---|---|
| OBP1 | 159 aa | NP_001153526.1 |
| OBP2 | 243 aa | NP_001153528.1 |
| OBP3 | 141 aa | NP_001153529.1 |
| OBP4 | 193 aa | NP_001153530.1 |
| OBP5 | 221 aa | NP_001153531.1 |
| OBP6 | 160 aa | NP_001153532.1 |
| OBP7 | 155 aa | NP_001153533.1 |
| OBP8 | 162 aa | NP_001153534.1 |
| OBP9 | 165 aa | NP_001153535.1 |
| OBP10 | 143 aa | NP_001153525.1 |
| OBP11 | 141 aa | XP_008178459.1 |
| OR1 (ORCO) | 463 aa | AQS60741.1 |
| OR2 | 403 aa | AQS60742.1 |
| OR4 | 368 aa | ARJ54248.1 |
| OR5 | 367 aa | KX890157.1 |
| OR10 | 369 aa | AQS60745.1 |
| OR17 | 430 aa | AQS60746.1 |
| OR20 | 420 aa | AQS60747.1 |
| OR22c | 403 aa | XP_003245950.2 |
| OR39 | 426 aa | AQS60753.1 |

Sequence Alignment and Transmembrane Domain Prediction

Sequence alignments were performed using Cluster Omega and the PRALINE server. Conservation mapping was performed using ConSurf. Transmembrane domain predications of the receptor proteins were performed using a consensus approach and four different serves—HMMTOP, TMpred, PHOBIUS and TMHMM. Alignments and conservation maps were analysed in GeneDoc, and phylogenetic trees were generated in FigTree v1.4.3.

For odorant-binding proteins, protein structures were initially predicted using the iTASSER database, which takes a hierarchical approach by identifying structural templates from the Protein Data Bank. All predicted protein structures were minimised using the Yasara minimisation server.

For odorant-receptors, Homology based modelling was performed using the olfactory receptor co-receptor, ORCO, from Apocrypta bakeri as a template. The structure of AbakORCO was obtained from the Protein Data Bank (PDB ID 6C70; resolution 3.5 Å). Pairwise sequence alignment was performed using PRALINE and the predicted transmembrane domains were manually aligned and annotated. The available ORCO structure shares a generally low sequence identity with the A. pisum odorant receptors, however, the general seven transmembrane structure should be conserved. The pairwise alignment served as a template for homology modelling using MODELLER 9.3 with loop refinement. The secondary structure of long extracellular loops 2 and 3 (EL2 and EL3) were individually predicted using the iTASSER server. Approximately 25 models were generated for each protein, and these were subsequently assessed using discrete optimised protein energy (DOPE) from MODELLER, in addition to Ramachandran and Z-score analysis, performed in PROCHECK and ProSA respectively.

All protein structures were visualised in PyMol 2.3.4.

Ligand Screening

Ligands were prepared in Chem3D 16.0 and AutoDock 4.2 (Python Molecule Viewer), then screened against computer generated models using AutoDock4.2 and the Racoon virtual screening tool. A Lamarckian Genetic Algorithm was selected for the simulation.

OBPs and ORs were all screened against a wide range of ligands. The binding energy of the complex and subsequent Kᵢ were calculated.

Molecular Dynamics

Molecular dynamics simulations were performed using GROMACS 2019. For odorant-binding proteins, the OLPS force field was used, and for odorant-receptors, a modified Gromas 53a6 force field was utilised.

For odorant-binding proteins, models were solvated and neutralised with ions (for negative Cl⁻, for positive Na⁺) before energy minimisation and equilibration (temperature and pressure) calculations were performed. For each protein, a 1 ns simulation was performed. The stability of the protein was then assessed by observing temperature and pressure stabilisation of the simulated models.

Molecular dynamics simulations were performed using the GROMACS 9.3 package. For OBPs, and OLPS force field was used, whereas for odorant-receptors, the Gromos53a6 forcefield was utilised, with modifications made to the Gromos53a6 forcefield parameters to include lipids and lipid topology. Lipid bilayer topology and structure were obtained from http://wcm.ucalgary.ca/tielemanklownloads. Receptor models were embedded into a lipid bilayer of 128 dipalmitoylphosphatidylcholine (DPPC) molecules. All models were solvated with explicit solvent (SPC) water and neutralised with either Cl⁻ or Na⁺ ions. Steepest-descent methods were used to minimise the energy of each system, at a reference temperature of 300 K. All bonds were constrained with LINCS and long-range electrostatics were handled using PME.

TABLE 2
Aphid sex pheromone components and analogues were screened
against wild type Acyrthosiphon pisum OBP6.

| | | Binding energy | Kᵢ |
|---|---|---|---|
| Example | Compound | (kcal mol⁻¹) | (μM) |
| Example 1 | 1 | −8.76 | 0.38 |
| Example 2 | 2 | −7.75 | 2.08 |
| Example 3 | 3 | −7.77 | 2.01 |
| Example 4 | 7 | −7.87 | 1.69 |
| Example 5 | 8 | −8.15 | 1.06 |
| Example 6 | 10 | −7.78 | 1.99 |
| Example 7 | 12 | −8.05 | 1.26 |
| Example 8 | 13 | −8.29 | 0.83 |
| Example 9 | 16 | −8.45 | 0.64 |
| Example 10 | 17 | −8.04 | 1.27 |
| Example 11 | 19 | −8.39 | 0.71 |
| Example 12 | 24 | −8.24 | 0.91 |
| Example 13 | 26 | −7.84 | 1.79 |
| Example 14 | 27 | −7.88 | 1.65 |
| Example 15 | 28 | −8.02 | 1.31 |
| Example 16 | 29 | −8.24 | 0.91 |
| Example 17 | 30 | −7.86 | 1.71 |
| Example 18 | 31 | −8.10 | 1.14 |
| Example 19 | 32 | −8.12 | 1.10 |
| Example 20 | 33 | −7.77 | 2.01 |
| Example 21 | 34 | −7.91 | 1.57 |
| Example 22 | 35 | −8.14 | 1.07 |
| Comparative | A | −7.67 | 2.37 |
| Example 1 | | | |
| Comparative | B | −7.52 | 3.07 |
| Example 2 | | | |
| Comparative | C | −7.69 | 2.30 |
| Example 3 | | | |
| Comparative | D | −7.60 | 2.69 |
| Example 4 | | | |
| Comparative | E | −7.00 | 7.38 |
| Example 5 | | | |

Structures of Compounds 1, 2, 3, 7, 8, 10, 12, 13, 16, 17, 19, 24 and 26-35, Compound A ((1R,4aS,7S,7aR)-nepetalactol), Compound B ((4aS,7S,7aR)-nepetalactone), Compound C ((1S,4aR,7R,7aS)-nepetalactol), Compound D ((4aR,7R,7aS)-nepetalactone) and Compound E ((1R,2S,5S)-dolichodial) are provided below.

Structures of Compounds 1, 2, 3, 7, 8, 10, 12, 13, 16, 17, 19, 24 and 26-35 and Compounds A-E:

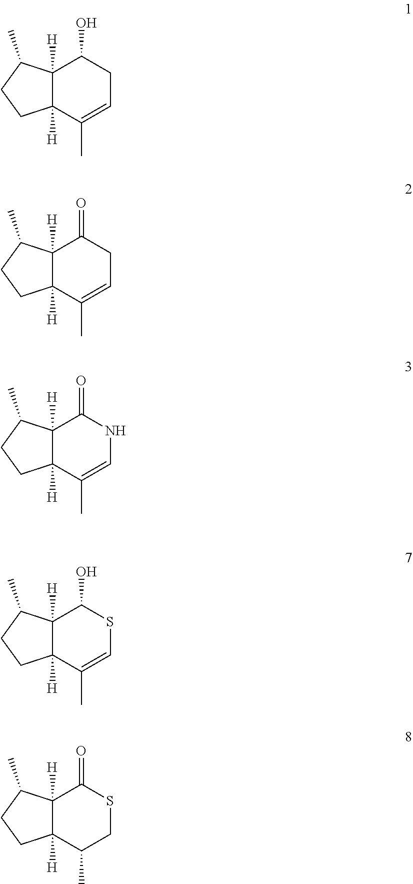

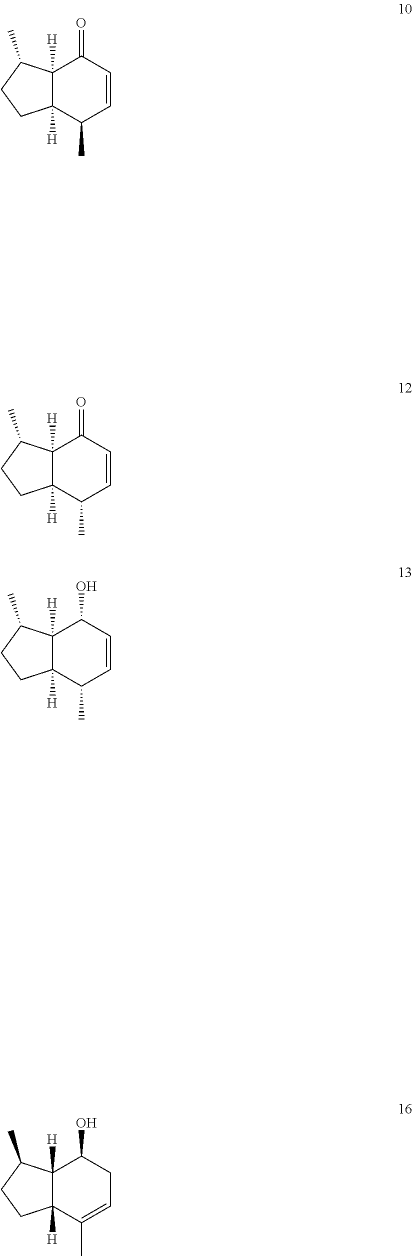

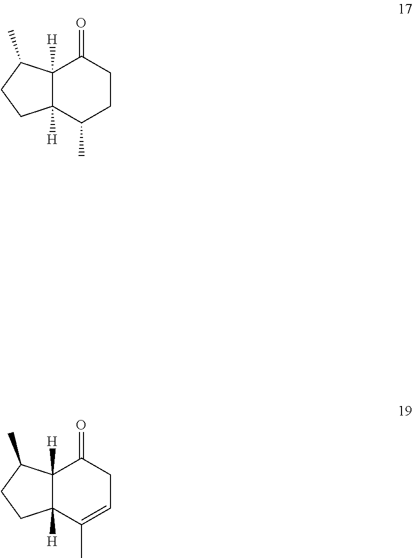

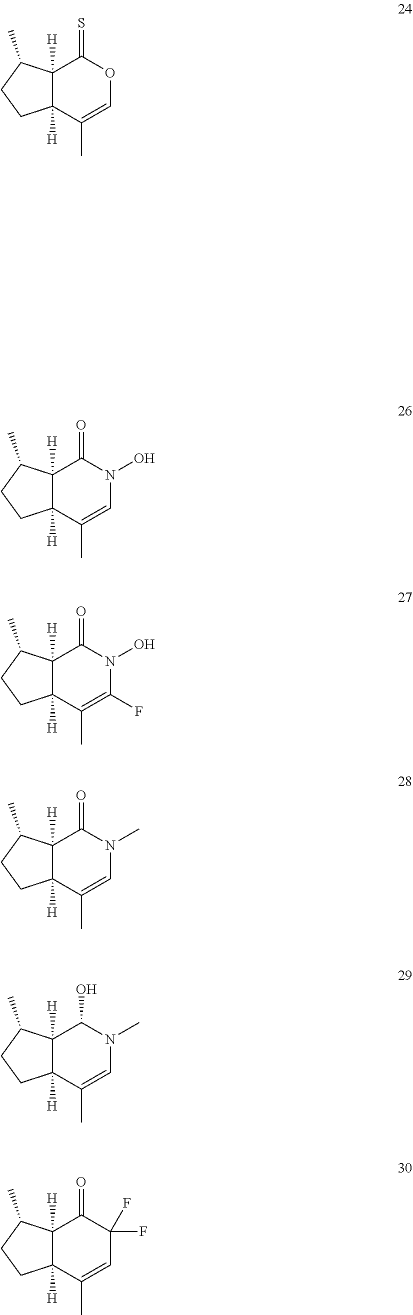

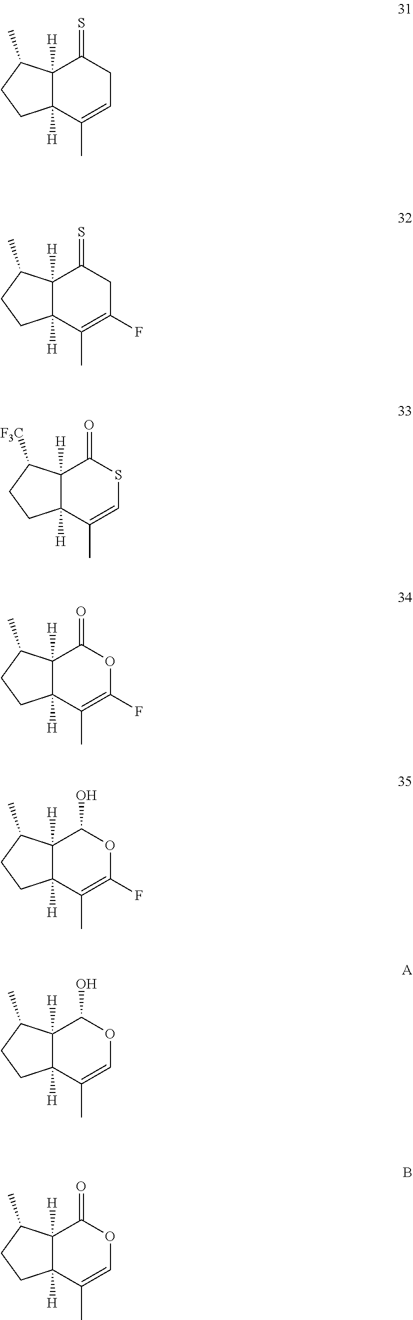

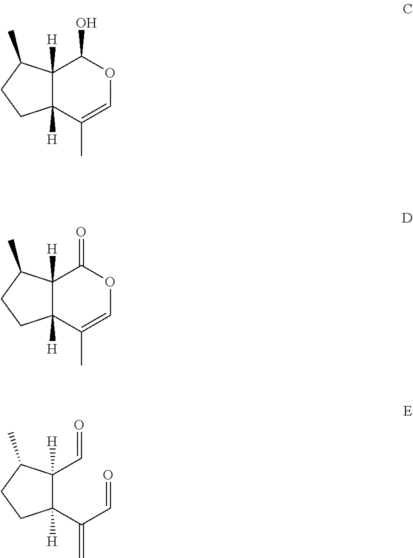

Referring to Table 2, the compounds of Examples 1 to 22 have a strong binding affinity to wild type Acyrthosiphon pisum OBP6. The compounds of Examples 1 to 22 also have high binding affinity compared with the compounds of Comparative Examples 1 to 5.

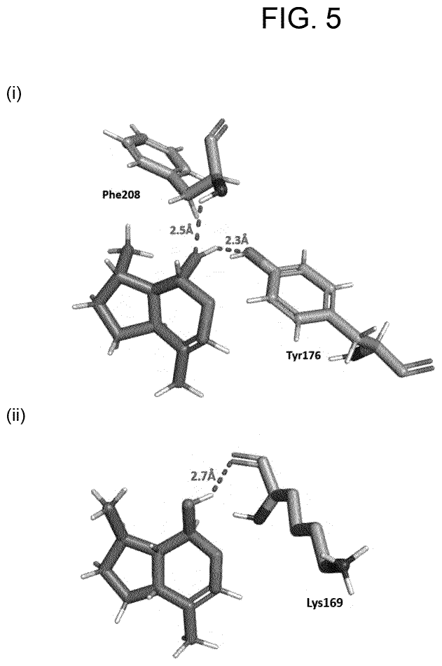

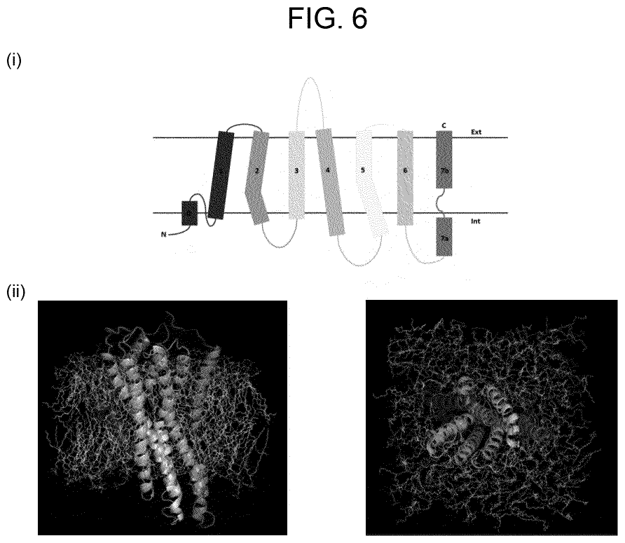

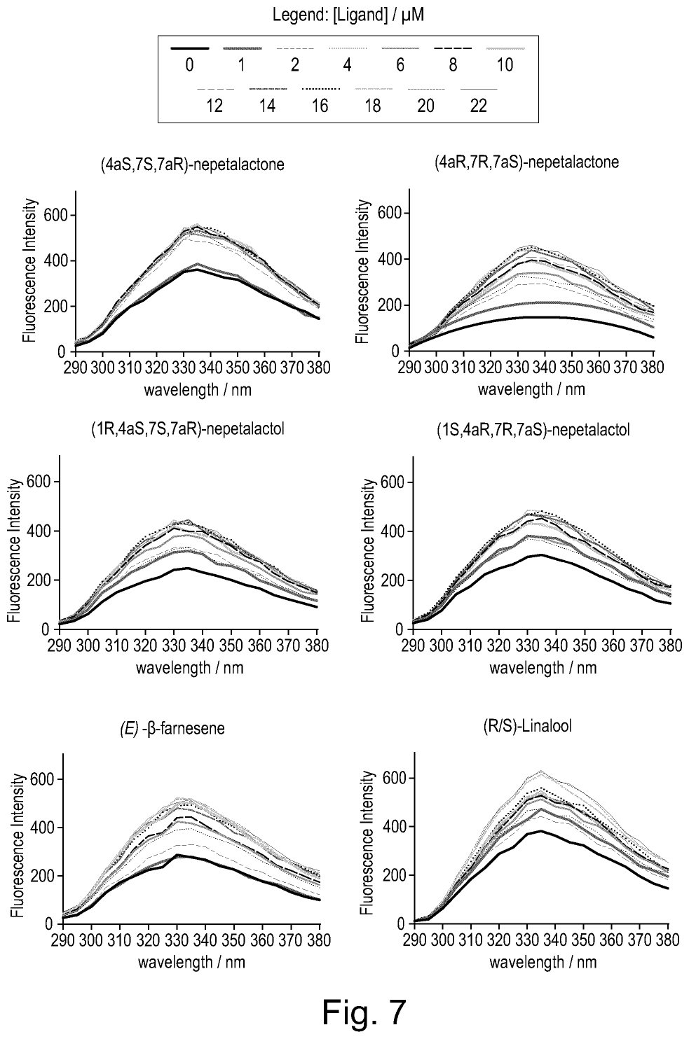

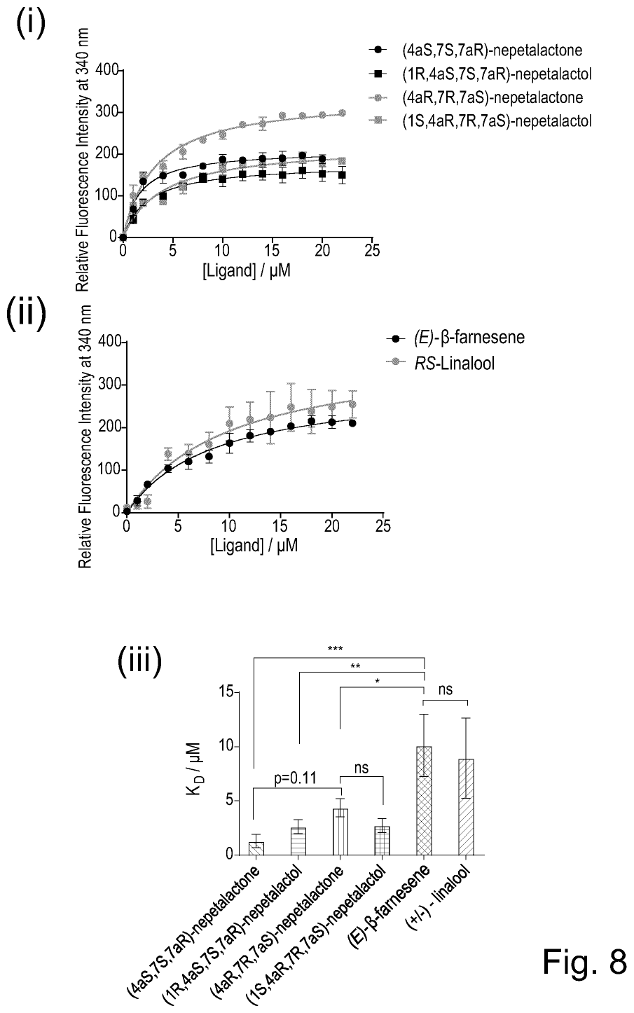

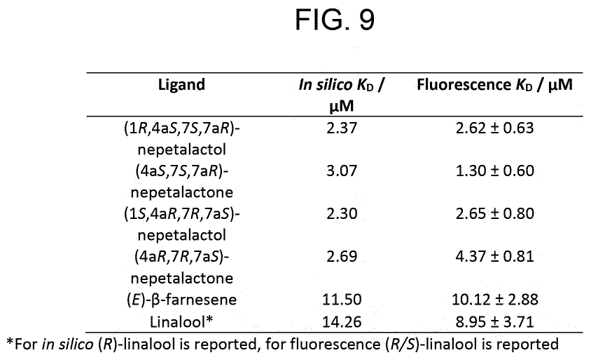

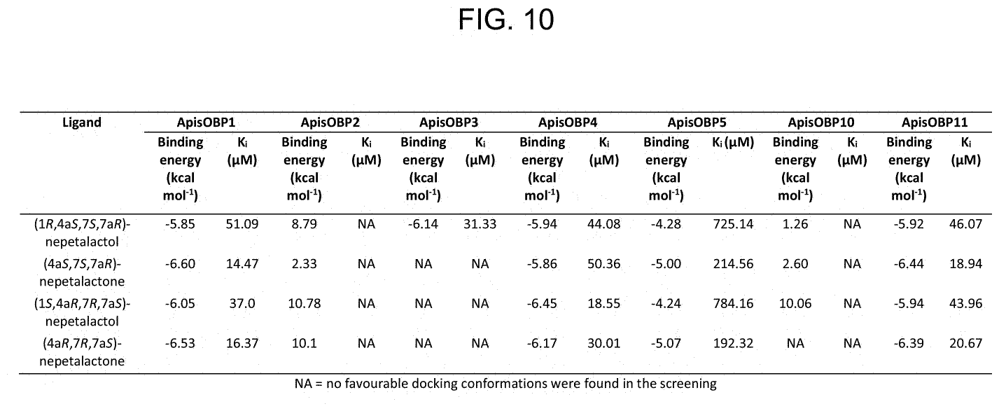

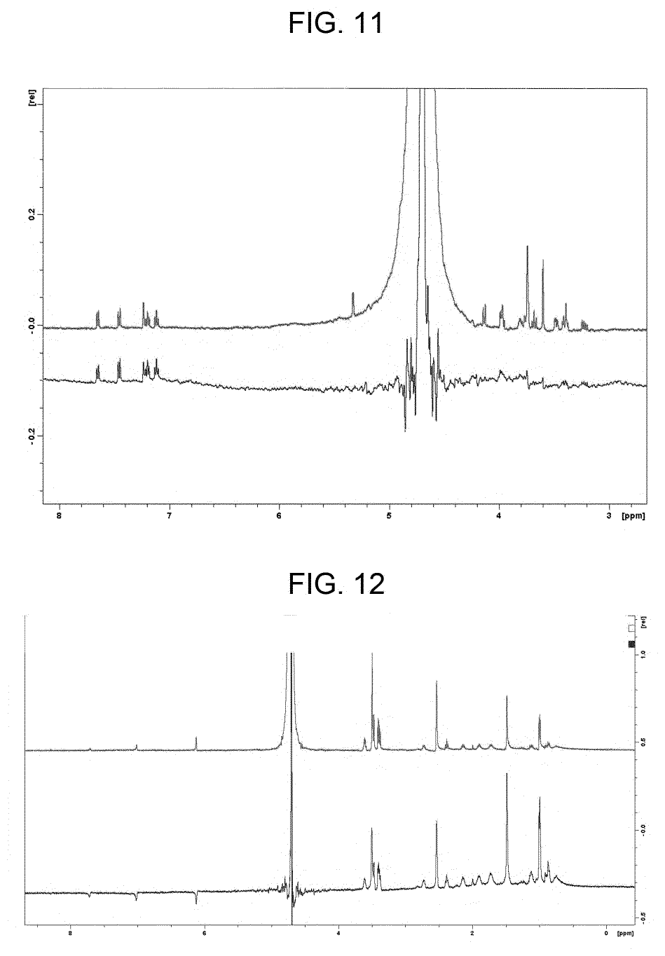

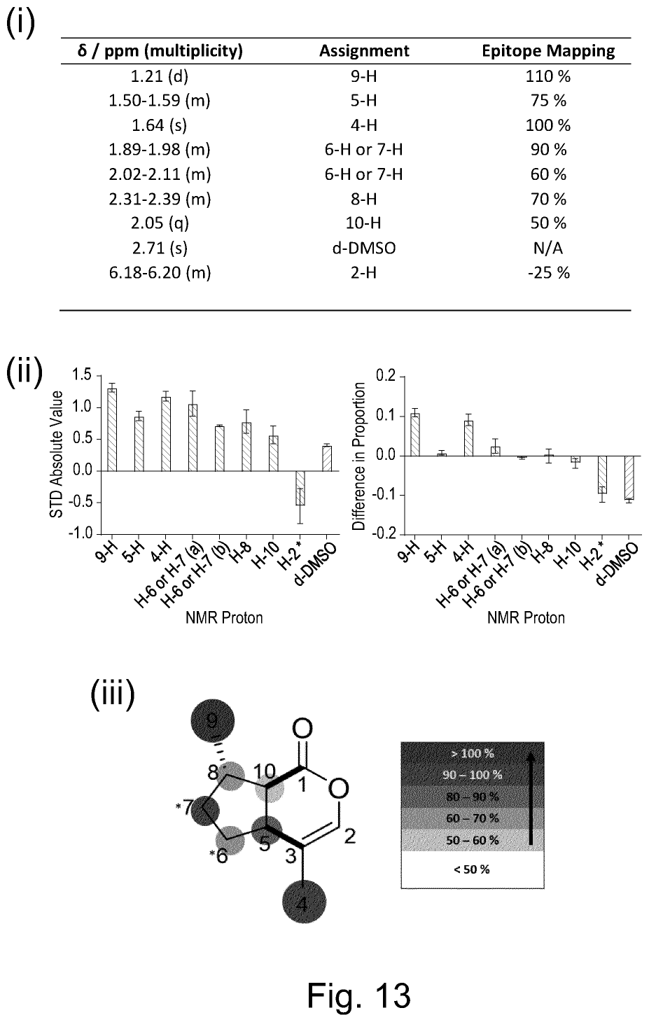

FIG. 5(i) shows that the lone pair on the oxygen atom within the six-membered ring of natural nepetalactone is held close to another oxygen lone pair on Tyr176. Without wishing to be bound by theory, it is postulated that a change from an oxygen atom to a carbon, nitrogen or sulfur atom (e.g. Examples 1-11 and 13-20) helps to increase binding affinity as there is less electrostatic repulsion (in the case of carbon and nitrogen, the lone pair is removed; in the case of sulfur, the lone pair is more diffuse with a less electronegative sulfur atom). It is also postulated that in the case of sulfur, the larger size of the sulfur atom leads to puckering of the ring system away from the oxygen lone pair on Tyr176, further enhancing binding affinity. Other structural changes (e.g. in Examples 12, 21 and 22) also help to increase binding affinity.

### Example 23: Docking of Aphid Sex Pheromones with Wild Type Acyrthosiphon Pisum OBPs and ORs

TABLE 3
Aphid sex pheromone components were screened against
wild type Acyrthosiphon pisum OBPs.

| | Binding energy (kcal mol⁻¹) of OBPs | | | | | | | | | | |
|---|---|---|---|---|---|---|---|---|---|---|---|
| Ligand | 1 | 2 | 3 | 4 | 5 | 6 | 7 | 8 | 9 | 10 | 11 |
| A | −5.85 | 8.79 | −6.14 | −5.94 | −4.28 | −7.67 | −5.99 | −5.89 | −5.31 | 1.26 | −5.92 |
| B | −6.60 | 2.33 | NA | −5.86 | −5.00 | −7.52 | −6.03 | −6.15 | −5.79 | 2.60 | −6.44 |
| C | −6.05 | 10.78 | NA | −6.45 | −4.24 | −7.69 | −6.00 | −6.38 | −5.47 | 10.06 | −5.94 |
| D | −6.53 | 10.1 | NA | −6.17 | −5.07 | −7.60 | −5.98 | −6.33 | −5.71 | NA | −6.39 |

(NA = no favourable docking conformations were found in the screening)

TABLE 4
Aphid sex pheromone components were screened
against wild type Acyrthosiphon pisum ORs.

Binding energy (kcal mol⁻¹) of ORs

| Ligand | 2 | 10 | 17 | 20 | 22c | 39 |
|---|---|---|---|---|---|---|
| A | −6.8 | −6.31 | −7.19 | −7.25 | −6.82 | −6.32 |
| B | −6.8 | −7.02 | −7.2 | −7.28 | −6.85 | −6.72 |
| C | −6.83 | −6.68 | −7.38 | −7.19 | −6.52 | −6.66 |
| D | −6.84 | −6.75 | −7.26 | −7.39 | −6.81 | −6.68 |

Referring to Table 3, OBP6 may have strong binding affinity to the aphid sex pheromone components, for both the biologically active enantiomers and the inactive enantiomers.

### Example 24: Expression and Purification of OBPs

Media Recipes

Media for use in molecular biology experiments were prepared as in Table 5.

TABLE 5
Media recipes for LB, 2xYT and LB agar.

| Media | Components (+1L H₂O) |
|---|---|
| LB liquid media | 8 g Tryptone, 5 g Yeast Extract, 5 g NaCl |
| 2xYT liquid media | 16 g Tryptone, 10 g Yeast Extract, 5 g NaCl |
| LB agar | 30 g LB agar mix (without NaCl), 5 g NaCl |

Media was sterilised by autoclaving at 120° C., then cooled before adding an appropriate antibiotic to a working concentration (Table 6). Agar was kept at 60° C. until poured into plates and allowed to set.

TABLE 6
Working concentrations of antibiotics added to media.

| Antibiotic | Working Concentration/μg ml⁻¹ |
|---|---|
| Ampicillin | 100 |
| Kanamycin | 50 |

Transformation of E. coli BL21(DE3) Competent Cells with A. pisum OBP Plasmids

All genes were previously cloned into a vector plasmid, pET45b or pET15b, containing an ampicillin resistance cassette, or pNIC28-Bsa4, containing a kanamycin resistance cassette. Each plasmid contained a hexa-histidine (His₆) tag encoded at the N-terminus of the protein, to allow for nickel affinity purification.

To transform cells, 5 μL of plasmid was added to BL21(DE3) competent E. coli cells and cooled to 0° C. The mixture was allowed to sit for 30 minutes, after which it was heat-shocked at 42° C. for 70 sec and cooled to 0° C. for a further 5 minutes. LB liquid media (800 μL) was added to the sample, followed by incubation (250 rpm; 37° C.) for 45 minutes. The sample was centrifuged (4000 rpm) for 2 minutes and the pellet resuspended in LB media (100 μL). The resuspension was spread onto an LB-agar plate containing the appropriate antibiotic and incubated (37° C.) for 12 hours. A clonal cell template for PCR was made from the overnight culture by diluting a scraping from a small, single colony in H₂O (10 μL).

Polymerase Chain Reaction (PCR)

A mixture for PCR was made by combining the cell template (1 μL), appropriate forward and reverse primers (1 μL of each), H₂O (7 μL) and a Taq polymerase mix (10 μL) containing dNTP, Taq polymerase and PCR buffer (100 mM Tris-HCl, 500 mM KCl, 15 mM MgCl₂, pH 8.3). The mixture was heated at 94° C. for 5 minutes, followed by 25 cycles of heating at 94° C. for 30 seconds, to 50° C. for 30 seconds, then 72° C. Finally, the mixture was taken to 72° C. for 30 minutes and stored at 4° C. Gel electrophoresis with a 1% agarose gel was used to check the PCR product and determine whether the transformation was successful.

Recombinant E. coli BL21(DE3) Starter Culture

A scraping from clonal culture or glycerol stock sample (500 μL) was added to 2×YT liquid media (10 mL) and incubated overnight (37° C.; 250 rpm) to generate a starter culture of E. coli BL21(DE3).

Protein Expression Test from Recombinant BL21(DE3) E. coli

Starter culture of recombinant BL21(DE3) E. coli cells (500 μL) was added to 2×YT liquid media containing the appropriate antibiotic (10 mL) and incubated (37° C.; 250 rpm) until the OD₆₀₀ values reached 0.5-0.6. Isopropyl β-D-1-thiogalactopyranoside (IPTG; 10 μL; 1 M) was added to induce expression, and the mixture incubated for a further 3-4 hours (37° C.; 250 rpm).

A sample of the culture (2 mL) was removed and centrifuged (12000 rpm; RT) for 2 min, and the cell pellet resuspended in SDS-PAGE loading buffer (6004). Samples were then denatured at 95° C. for 10 minutes, before loading onto an SDS-PAGE gel.

Large Scale Expression and Refolding of OBPs

2×YT liquid media (600 mL) was inoculated with starter culture (3 mL) and incubated (37° C., 250 rpm) until an OD₆₀₀ of 0.7-0.8 was reached. IPTG (300 μL; 1 M) was added to induce expression and the mixture incubated for a further 3-4 hours (37° C., 250 rpm).

Cells were harvested by centrifuging for 15 minutes (3500 rpm).

The cell pellet was resuspended in ice-cold TBS buffer (10 mL; 25 mM Tris, 500 mM NaCl, pH 8.0) and incubated on ice for 10 minutes. The mixture was sonicated for 4 minutes on ice, then centrifuged for 30 minutes (35000 rpm, 4° C.). The pellet was resuspended in TBS+0.2% Triton X-100 (10 mL). The sonication and centrifugation steps were repeated, and the pellet resuspended in 8M urea (1.5 mL) and 10 mM 1,4-dithiothreitol in 100 mM Tris (1.5 mL; pH 8). The mixture was incubated for 1 hour at room temperature, then rapidly diluted with 27 mL TBS+5:0.5 mM GSH:GSSG. The diluted mix was incubated overnight at RT.

The overnight mixture was centrifuged for 10 minutes (3500 rpm) and the supernatant filtered through a 0.22 μm filter.

Purification of OBPs

OBPs were purified using Histrap® columns, preconditioned with 25 mM imidazole buffer. The filtrate from the refolding step (Chapter 3; 3.2.5) was passed through the column, leaving the histidine-tagged protein bound to the nickel. An imidazole buffer (500 mM) was used to displace the protein and fractions were collected and analysed using SDS-PAGE.

Sodium Dodecyl Sulfate Polyacrylamide Gel Electrophoresis (SDS-PAGE)

The resolving gel (1.0 mm width; 5 mL) was prepared as in Table 7. Approximately 1.5 mL of 2-isopronaol was added to remove bubbles and the gel left to set. After 20 minutes, the isopropanol was removed, and the stacking gel added. A comb was used to form wells before leaving the gel to set for a further 20 minutes.

TABLE 7
Composition of resolving and stacking gels for SDS-PAGE.

Volume/μL

| | Resolving gel | Stacking Gel |
|---|---|---|
| | (15%; 5 mL) | (3%; 2 mL) |
| H₂O | 1770 | 1185 |
| 40% acrylamide | 1875 | 150 |
| 1.5M Tris buffer (pH 8.8) | 1250 | 0 |
| 0.5M Tris buffer (pH 6.8) | 0 | 250 |
| 10% SDS buffer | 50 | 25 |
| 10% ammonium persulfate* | 50 | 25 |
| TEMED* | 5 | 3 |

(*added last to catalyse polymerisation)

Samples for the gel (20 μL) were prepared by combining protein samples (10 μL) with sample buffer (10 μL; prepared as in Table 8). Samples were run alongside an appropriate marker (11-245 kDa). The gel was run at 200 V for 90-120 minutes and GelCode™ Blue Stain (Thermo-Fisher Scientific) was added for approximately 1 hour to resolve the protein bands.

TABLE 8
Composition of SDS-PAGE Protein Sample Buffer

| Reagent | Concentration |
|---|---|
| Tris | 80 mM |
| SDS | 2.0% |
| Glycerol | 10% |
| Bromophenol blue | 0.0006% |
| DTT | 0.1M |

Histidine-Tag Cleavage from Purified Protein

To remove the hexa-histidine tag from purified proteins, an appropriate cleavage enzyme was used (Table 9). The selection of the enzyme depended on the cleavage site encoded in the original vector plasmid. The protein was buffer-exchanged in CaCl₂ in TBS buffer (2 mM CaCl₂, 25 mM Tris; 500 mM NaCl), and enzyme added. Cleavage was monitored by observing a change in mass using mass spectrometry. After completion, excess tagged protein was removed by passing the mixture through a Histrap® column and collecting the eluent.

TABLE 9
Vector plasmids used for transformation and
their His-tag cleavage sites and enzymes.

| | | | Cleavage |
|---|---|---|---|
| Plasmid | His-Tag FASTA sequence | Cleavage Site | Enzyme |
| pET45b | MAHHHHHHVG TGSNDDDDKSPDP | DDDDK/S | Enterokinase |
| | (SEQ ID NO: 30) | (SEQ ID NO: 33) | |
| pNIC28-Bsa4 | MHHHHHHSSGVDLGTENLYFQS | ENLYFQ/S | TEV (Tomato |
| | (SEQ ID NO: 31) | (SEQ ID NO: 34) | Etch Virus) |
| pET15b | MGSSHHHHHHSSGLVPRGSH | GLVPR/GS | Thrombin |
| | (SEQ ID NO: 32) | (SEQ ID NO: 35) | |

Plasmid Extraction, Purification and Sequencing

Plasmids were extracted using a GeneJET Plasmid Miniprep Kit (Thermo-Fisher Scientific) and sequenced by Queen's Medical Centre laboratories, University of Nottingham or Eurofins (UK).

Protein Buffer Exchange and Concentration

All proteins were buffer exchanged and desalted using a PD-10 desalting column and concentrated using a Vivaspin 20 (10 kDa MWCO).

Site-Directed Mutagenesis

Site-directed mutagenesis (SDM) of OBP6-His₆ was performed to insert a thrombin tag. A Q5 Site-Directed Mutagenesis kit (New England Biolabs) was used to perform the mutagenesis. Primers (Table 10) were obtained from Sigma-Aldrich.

TABLE 10
Forward and reverse primers for
site-directed mutagenesis (thrombin
cleavage site insertion) of OBP6-His₆
Primers for thrombin cleavage site insertion

| | |
|---|---|
| Forward | cgcggcagcGCTGGGTACGATAGAACATG |
| Reverse | cggcaccagCGGATCCGGACTCTTGTC |

Samples for PCR were prepared by combining plasmid containing the gene of interest (1 μL), forward and reverse primers (1 μL of each), H₂O (9 μL) and Q5 hot start master mix (12.5 μL). The mixture was heated at 98° C. for 30 seconds, followed by 25 cycles of heating at 98° C. for 10 seconds, to 64° C. for 20 seconds, then 72° C. for 2 minutes. Finally, the mixture was taken to 72° C. for 2 minutes and stored at 4° C.

The PCR product (1 μL) was mixed with KLD reaction buffer (5 μL), KLD enzyme mix (1 μL) and H₂O (3 μL) and left at RT for 5 minutes. Finally, the mixture (5 μL) was used to transform NEB 5-alpha Competent E. coli cells as described under “Transformation of E. coli BL21(DE3) Competent Cells with A. pisum OBP Plasmids”. Results were validated by sequences as described under “Plasmid Extraction, Purification and Sequencing”.

Fast-Protein Liquid Chromatography

To purify proteins, gel filtration via fast-protein liquid chromatography (FPLC) was performed using an Akta FPLC. The FPLC was fitted with a Superdex size-exclusion column, and samples were exchanged into a TBS buffer (Tris 15 mM, NaCl 250 mM; Table 11). Protein samples were collected using an autosampler and subsequently concentrated as described under “Protein Buffer Exchange and Concentration”.

TABLE 11
Run conditions for size exclusion fast-
protein liquid chromatography (FPLC).

| Step | Time | | Flow Rate | Buffers |
|---|---|---|---|---|
| Wash | 20 | min | 3.0 mL min⁻¹ | 1:1 20% EtOH:H₂O |
| Wash | 20 | min | 3.0 mL min⁻¹ | 1:1 TBS:H₂O |
| Equilibrate | 40 | min | 1.5 mL min⁻¹ | TBS |

Inject Sample

| | | | | |
|---|---|---|---|---|
| Run | 60-180 | min | 1.5 mL min⁻¹ | TBS |
| Wash & Store | 40 | min | 3.0 mL min⁻¹ | 20% EtOH |

Mass Spectrometry Analysis

To confirm efficient cleavage of the His-tag and determination of the structure, denatured mass-spectrometry of the recombinant proteins was performed using a Waters QTof2 spectrometer. Samples were prepared using a ZipTip® before injected into the mass spectrometer.

General Mass Spectrometry

All mass spectrometry was undertaken using electrospray ionisation and conducted on the QTof2 or QTof3 under denaturing conditions (80% CH₃CN, 0.1% formic acid) and the settings detailed in Table 12, unless otherwise stated.

Mass spectra were analysed using MassLynx 3.0 software.

TABLE 12
Denatured mass-spectrometry settings for the QTof 3.0

| Setting | Value | |
|---|---|---|
| Capillary Voltage | 2.80 | kV |
| Cone Voltage | 35 | V |
| Desolvation Temperature | 80° | C. |
| Source Temperature | 50° | C. |

| | |
|---|---|
| Rf Lens | 1.0 |

Sample Preparation Using the ZipTip®

Samples for mass-spectrometry were prepared by ZipTip®. The ZipTip® was conditioned by washing with elution buffer (80% CH₃CN, 0.1% formic acid; 5×10 μL), followed by equilibrating with wash buffer (5% MeOH, 0.1% trifluoroacetic acid; 7×10 μL). Trifluoroacetic acid (TFA) was added to the sample to a concentration of 0.1%, which was adsorbed onto the column (20 aspirations). The sample was then washed with wash buffer (20×10 μL) and eluted into elution buffer (10 μL, 20 aspirations).

### Example 25: Fluorescence Based Assays of A. pisum Odorant-Binding Proteins and Interactions with Ligands

Fluorescence Measurements

All fluorescent measurements were undertaken using a Perkin-Elmer LS50B fluorescence spectrophotometer, using a 2 mL quartz cuvette, and the settings described below in Table 13, unless otherwise stated. Spectra were recorded using FL WinLab software.

TABLE 13
Settings for fluorescence measurements using the
Perkin-Elmer LS50B fluorescence spectrophotometer

| | | |
|---|---|---|
| | Intrinsic | Probe |
| | Fluorescence | Fluorescence |
| Setting | (Tryptophan) | (1-NPN) |

| | | | | |
|---|---|---|---|---|
| Excitation | 280 | nm | 337 | nm |
| Wavelength | 290-400 | nm | 350-600 | nm |
| Emission | | | | |
| Wavelengths | | | | |
| Excitation Slit | 5.0 | nm | 5.0 | nm |
| Emission Slit | 5.0 | nm | 5.0 | nm |

Ligand Binding and Saturation Curves

Saturation of OBPs with fluorescent probe and 1-NPN (Sigma-Aldrich) was initially measured by titrating a 2 μM protein sample (2 mL in 25 mM Tris-HCl) with aliquots of 1 mM ligand (Sigma-Aldrich) in methanol to final concentrations of 1-16 μM. The fluorescence intensity at 330 nm was recorded.

The competitive binding of ligands was measured by observing the intrinsic fluorescence of tryptophan. Titrations were performed with aliquots of 1 mM ligand in methanol to final concentrations of 1-20 μM, either after the addition of fluorescent probe to a final concentration of 1 μM or in the absence of fluorescent probe.

TABLE 14
The calculated K_{D} values for OBP6 and various
ligands from different binding assays.

| | OBP6 | | OBP9 |
|---|---|---|---|
| | 1-NPN Assay | Fluorescent Probe- | 1-NPN Assay |
| Ligand | K_{D}/μM | Free Assay K_{D}/μM | K_{D}/μM |
| (1R,4aS,7S,7aR)- | 2.62 ± 0.63 | 12.74 ± 2.31 | 5.74 ± 1.71 |
| nepetalactol | | | |
| (4aS,7S,7aR)- | 1.30 ± 0.60 | 1.90 ± 0.35 | 6.49 ± 1.58 |
| nepetalactone | | | |
| (1S,4aR,7R,7aS)- | 2.65 ± 0.80 | 8.46 ± 1.62 | 6.29 ± 1.99 |
| nepetalactol | | | |
| (4aR,7R,7aS)- | 4.37 ± 0.81 | 12.01 ± 4.24 | 9.68 ± 4.57 |
| nepetalactone | | | |
| (E)-β-farnesene | 10.12 ± 2.88 | 34.47 ± 10.85 | 8.32 ± 2.67 |
| (R/S)-linalool | 8.95 ± 3.71 | NA | NA |

(NA = not available or not measured)

Analysis of Fluorescence data

To generate K_{D} values, relative fluorescence intensity was plotted against the concentration of ligand as a binding curve. K_{D} values were generated in GraphPad Prism 7 using a non-linear regression and the inbuilt equation:

$$y = \frac{{B}_{max} ⋆ x}{{K}_{D} + x}$$

Each calculated K_{D} value had an associated error from the non-linear regression. To account for these errors in statistical analysis, values were weighted in direct proportion to their error—the higher the error, the lower the weighting of the values. The ‘weight’ factor was calculated using the following equation:

$$weight = \frac{1}{{( S E )}^{2}}$$

Statistical analysis of fluorescence data was performed using R 3.4.4. A one-way weighted ANOVA was performed between ligands for each protein, and a two-way weighted ANOVA was performed to investigate the interactions between proteins and ligands. A Tukey Test was used for post-hoc analysis (Table 15).

TABLE 15
Statistical analysis results for a weighted analysis
of variance (ANOVA) of the binding affinities (K_{D})
difference between different ligands and OBP6.

| | Interaction | | |
|---|---|---|---|
| Assay | Ligand A | Ligand B | p-value |
| 1-NPN | (1R,4aS,7S,7aR)- | (E)-β-Farnesene | 0.0021 |
| | Nepetalactol | | |
| | (4aS,7S,7aR)- | | 0.00017 |
| | Nepetalactone | | 0.010 |
| | (1S,4aR,7R,7aS)- | | |
| | Nepetalactol | | |
| | (4aR,7R,7aS)- | | 0.015 |
| | Nepetalactone | | |
| | (1R,4aS,7S,7aR)- | (±)-Linalool | 0.023 |
| | Nepetalactol | | 0.0014 |
| | (4aS,7S,7aR)- | | |
| | Nepetalactone | | |

The interaction between (4aS,7S,7aR)-nepetalactone and (4aR,7R,7aS)-nepetalactone did not give a significant difference, with a p-value of 0.11 in the 1-NPN assay and a p-value of 0.055 in the fluorescent probe free assay. A weighted t-test was subsequently performed, giving a significant difference with a p-value of 2.27×10⁻⁴.

### Example 26: Saturation Transfer Difference NMR Experiments

General NMR Spectroscopy

Samples were run using an AVANCE Bruker DRX-500 MHz Nuclear Magnetic Resonance spectrometer with a 5 mm BBO BB-1H probe and set at 500 MHz for 1H spectra and 125 MHz for ¹³C spectra. Analysis of Bruker data was performed using Topspin 4.0.7, and analysis of Varien data performed with CCPNMR V2 and NMRPipe. Standard NMR tubes were used with a sample volume of 600 μL unless otherwise stated. Deuterated chloroform (CDCl₃), methanol (CD₃OD) and dimethyl sulfoxide (d-DMSO) were stored over 4 Å molecular sieves and used as both sample solvents and internal standards. For assignment of small molecules, additional 2D-NMR spectroscopy experiments were performed.

Standard Sample Preparation

Protein samples for NMR were desalted and buffer exchanged into 9:1 H₂O:D₂O unless stated otherwise (as in “Protein Buffer Exchange and Concentration”). Ligand samples were not soluble in D₂O and samples were prepared in d-DMSO. This resulted in a final NMR solvent including H₂O, D₂O and d-DMSO.

Saturation Transfer Difference (STD) NMR

An initial test assay containing Bovine Serum Albumin (BSA) protein with tryptophan and sucrose was prepared for STD NMR. Samples were run for 192 scans, and the saturation transfer difference (STD) spectra generated.

TABLE 18
Composition of the final STD assays, both
the BSA test assay and OBP6 assay.

| Component | Test Assay | OBP6 Assay |
|---|---|---|
| Protein (Unlabelled) | BSA; 100 μM | OBP6; 30 uM |
| Suspected Binder | Tryptophan; 10 mM | (4aS,7S,7aR)- |
| | | nepetalactone); 3 mM |
| Suspected Non-Binder | Sucrose; 10 mM | Not Included |

STD absolute values were calculated by observing the change in proportions between the off-resonance spectrum and the final STD spectrum using the following equation:

$$STD absolute value = \frac{{I}_{0} - {I}_{STD}}{{I}_{0}}$$

in which the term (I₀−I_{STD}) represents the ratio of peak intensity in the STD spectrum and I₀ the ratio of intensity in the off resonance spectrum. A second value representing the proportionate change was calculated using the following equation:

Difference in proportion=I₀−(I₀−I_{STD})

Finally, epitope mapping was performed by calculating the relative peak integration in the STD spectrum vs the off-resonance spectrum of a peak as a percentage.

### Synthesis Examples 1-4

Nepetalactone and derivatives may be synthesized using (inverse electron demand) Diels-Alder reactions as described in Dawson et al. (Bioorganic Med. Chem. 1996, 4 (3), 351-361) and Schreiber et al. (J. Am. Chem. Soc. 1986, 108, 8274-8277). Some further modifications are illustrated below.

### Synthesis Example 1: (4aS,7S,7aR)-4,7-dimethyl-2,4a,5,6,7,7a-hexahydro-1H-cyclopenta[c]pyridin-1-one (Compound 3)

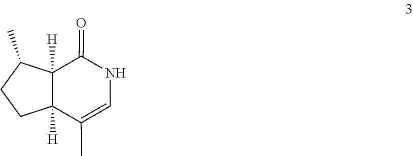

Ammonia gas was bubbled gently through a solution of nepetalactone (1.02 g, 6.12 mmol) in DCM (70 ml) for 1 hour. The reaction flask was then sealed and stirred for a further 16 hours. The crude reaction mixture was concentrated under vacuum and purified via distillation (Kugelrohr, 46 mbar, 162° C.) to yield the product. δ_{H} (500 MHz, CDCl₃) 6.73 (1H, s), 5.62-5.70 (1H, m), 2.71 (1H, q, J=8.4 Hz), 2.27-2.37 (2H, m), 1.98-2.08 (1H, m), 1.78-1.87 (2H, m), 1.64 (3H, s), 1.45-1.54 (1H, m), 1.21 (3H, d, J=6.6 Hz).

### Synthesis Example 2: (4aS,7S,7aR)-4,7-dimethyl-5,6,7,7a-tetrahydrocyclopenta[c]thiopyran-1(4aH)-one (Compound 23)

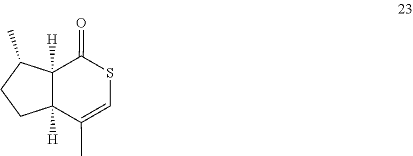

In order, indium triflate (0.17 g, 0.30 mmol), toluene (7.5 ml), nepetalactone (1.00 g, 6.02 mmol), sulphur (0.21 g, 6.62 mmol) and phenyl silane (0.43 g, 4.01 mmol), under N₂, was added to a screw topped flask, sealed and heated to 120° C. for 24 hours. The reaction mixture was cooled and the pressure released carefully. The reaction mixture was filtered through celite before being purified on silica gel (2% EtOAc in hexanes) to give the product (0.12 g, 12% yield) as yellow/brown oil. δ_{H} (500 MHz, CDCl₃) 5.72 (1H, s), 2.84-2.94 (1H, m), 2.55 (1H, hept, J=7.0 Hz), 2.38 (1H, t, J=7.5 Hz), 1.89-2.00 (2H, m), 1.87 (3H, s), 1.69-1.79 (1H, m), 1.17-1.32 (1H, m), 1.01 (3H, d, J=6.8 Hz).

### Synthesis Example 3: (4aS,7S,7aR)-4,7-dimethyl-5,6,7,7a-tetrahydrocyclopenta[c]pyran-1(4aH)-thione-nepetathionolactone (Compound 24)

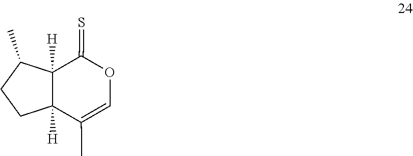

To a solution of nepetalactone (2.00 g, 12.03 mmol) in acetonitrile (30 ml) was added phosphorus pentasulphide (2.68 g, 6.02 mmol) followed by hexamethyldisiloxane (4.92 g, 30.30 mmol) and heated to 82° C. for 16 hours. The solvent was removed under vacuum and the crude product purified on silica gel (2% EtOAc in pet ether) to give the product (0.42 g, 37% yield) as a yellow oil. δ_{H} (500 MHz, CDCl₃) 6.37 (1H, s), 2.95 (1H, t, J=8.5 Hz), 2.70-2.77 (1H, m), 2.28 (1H, hept, J=7.4 Hz), 1.81-1.92 (2H, m), 1.72-1.81 (1H, m), 1.62 (3H, s), 1.28-1.33 (1H, m), 1.19 (3H, d, J=6.7 Hz).

### Synthesis Example 4: (4aS,7S,7aR)-4,7-dimethyl-1,4a,5,6,7,7a-hexahydrocyclopenta[c]pyran-1-thiol (Compound 25)

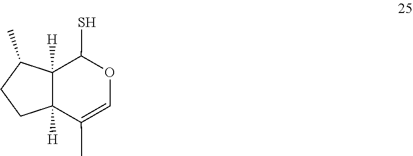

To a solution of nepetathionolactone (Compound 24) (0.35 g, 1.90 mmol) in isopropyl alcohol (45 ml) was added sodium borohydride (0.10 g, 2.66 mmol) and the mixture stirred for 2 hours. The reaction was quenched with 12 ml of 2M HCl and saturated brine was added. The aqueous layer was extracted with diethyl ether. The combined organic layer was dried (MgSO₄) and concentrated under vacuum. The crude product was purified on silica gel (15% Et₂O in pet ether) to give the product (0.21 g, 60% yield) as a pale yellow oil. δ_{H} (500 MHz, CDCl₃) 6.00 (1H, s), 4.23 (1H, d, J=4.3 Hz), 2.75 (1H, q, J=7.7 Hz), 1.80-2.32 (4H, m), 1.43-1.57 (1H, m), 1.79 (3H, s), 1.29-1.38 (1H, m), 0.91 (3H, d, J=6.7 Hz).

### Fabrication Example 1: Biosensor Comprising ApisOBP6

A sensing surface (a film bulk acoustic wave resonator—FBAR—fabricated by Sorex Sensors) was functionalised via the APTES-glutaraldehyde method. The sensing surface to be functionalised was exposed to a stream of ozone for 15 mins, before being allowed to stand in air for 30 mins and washed with distilled water. To the freshly oxidised sensing surface, a solution of APTES (2% in ethanol) was added and ensured the whole sensing surface was covered and allowed to stand for 10 mins. The excess reagent was removed with clean ethanol, before being dried under a stream of nitrogen and cured in an oven (110° C.) for 60 mins. After cooling to RT, a 1% solution of glutaraldehyde was added to the surface for a further 60 mins. A solution of the desired OBP (ApisOBP6, 1 μM) was added to the surface for 1 hour. The functionalised sensing surface was washed with distilled water and stored until required.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

SEQUENCE LISTING

SEQ ID NO: 1 (OBP1 from Acyrthosiphon pisum)
MLNLKVMMFLCLSVIVVYCESDQVPINSSAAVESCLLETNMTRDEFEDMLTSPNARELTILKSH
AHKCMFGCVMRKNHIVNDGVVSKEVLSKYVLNFYGRPDYKRRLIIKDVEHIVDVCAKKVADESE
TDECELAATLVTCIVLEANKAGLVDDPARQI
SEQ ID NO: 2 (OBP2 from Acyrthosiphon pisum)
MKVSAATAVLVALVATVQSSDPCNISTCYKSGTTKPPMAVTPTHLPVQSSSTQTSHPQTTYAKD
HVHGSTTTKSGVNATVTTASGASVNGTEPPAVVKSSAGVTGNSTTPKPTMTEGHVALKQKLNTI
AVKCKDELHAPQEIMALVSNTVVPQNEQQRCYLECVYKNLNLIKNNKFSVEDGKAMARIRFANQ
PEEHKKAVTIIETCEKEAVIDPKTTEKCAAGRVIRNCFVKNGEKINFFPKA
SEQ ID NO: 3 (OBP3 from Acyrthosiphon pisum)
MISSTFYITLVFGIAMLISCGHGRFTTEQIDYYGKACNASEDDLVVVKSYKVPTTETGKCLMKC
MITKLGLLNDDGSYNKTGMEAGLKKYWSEWSTEKIESINNKCYEEALLVSKEVVATCNYSYTVM
ACLNKQLDLDKST
SEQ ID NO: 4 (OBP4 from Acyrthosiphon pisum)
MRGNYSSMVFLLFAIGFQDIFCQKQEPSGKCRAPDKAPLNLEIIINTCQEEIKSALLQEALDIL
NDGNVEQNTPNYSSRSKREAEEDLTNEERRVAGCLLQCVYKKVKAVDETGFPVVDGLMKLYNEG
VQDRNYYIATLSAVRHCISIAQQLKQQQPSKSFDDGQTCDLAYEMFECVSEKIEENCGVENKSN
N
SEQ ID NO: 5 (OBP5 from Acyrthosiphon pisum)
MSANSATIKCIAVAAILLQISVIFADAGHHRRGKELLDTEDSDFFRCKQASRKSCCGPENAMKR
FGDKDKVAADECYAQVAEKFATVTATTPKQDLFSAEAVKITKKKQFCLHECIGKKNNLLTEDGS
LNKTFIADYAMKSVFKEQWQKQVGQKALDKCLEETYIPWPAEDKENVCNPVYVQFQHCLWLQYE
SNCPANKIKITKKCEKTRNRYRMQKSTSN
SEQ ID NO: 6 (OBP6 from Acyrthosiphon pisum)
MQKVVFICIFAIICQTVFTAGYDRTWILRQKRGTNDDECRTLLPSSEKKLPSCCQMPNILPNLD
STWEKCFETFKQFKDKPETKEYKEMAHGKEPPCLFQCIFMQSGLTTSDGKLNEDAITKKMSEGI
NNDEKWKSIWQNSLNKCFDDVKQEDKKQILIMNTPAGRLMKCFLRDMYMSCPKNVWVESSECLN
MKDLVQKCPEMPPPVFKSPPKLI
SEQ ID NO: 7 (OBP7 from Acyrthosiphon pisum)
MVARKRMYNMLPTTVLFAIIAATVLKDCDAYLSEAAIKKTQQMLKTVCSKKHSVEEDVFTNIKK
GIFPEDNNNIKCYFACNFKTMQLINQKGVIDKKMFKDKMSMMAPPNVYKILLPVIEQCTGKDKG
EELCQSSYNVIKCAHSVDPKSLEFLPL
SEQ ID NO: 8 (OBP8 from Acyrthosiphon pisum)
MFALKVACLCLSVAVVFGENNQQNGPSDRSATIFQSCIAETKLSGDALKGFRSMSIPKTQAEKC
MMGCLMRKVNVINKGKFSVEEATKVAQKYYGTNEAMMKKAKDLIDVCAKKAQSTTEECALAGIV
TTCIVEEAQKAGLSGGPGSRSRRTVSPKFRRDAM
SEQ ID NO: 9 (OBP9 from Acyrthosiphon pisum)
MIIKKTLLLSVFVLFGCLFSINKADDADAKDKELMSKLFTVVFKCFKDADWGTCGEMITTKYDI
TQAKYKQCTCHMACAGEELGMINASGQPEPAKFLEYVNKINNPDIKSQLQLIYDKCQNVKGSEK
CDLAEQFAICAFKESPALKERVSTLMEMLVKMKPKSK
SEQ ID NO: 10 (OBP10 from Acyrthosiphon pisum)
MEHLRSTNVVFAIVMALLVVQSSTRPQPDEMEEIKRTLYNACAGKFPITEEIKNNAKNSIISDD
PTFKCFLKCCFDEMSMIDEDGIIDGDSLKAMAPDHIKPILEQVIPSCTKNVKQDGCEASFEFIS
CGIKLNPLIVALLPL
SEQ ID NO: 11 (OBP11 from Acyrthosiphon pisum)
MSSSTFYITLLFGIAMLISCGYGIFTTEQIDYYGKACNASEDDLIVLKSYKVPSTETGKCLMKC
MITKLGLLNDDGSYNKTGMEAGLKKYWSEWATEKIETINEKCYEEGNTATLLYHVAIYFTCVSG
DYSDVQLLIHCDGMFEQEVGSRQVNLKLLIMLKIGLSEPKR
SEQ ID NO: 12 (ORCO from Acyrthosiphon pisum)
MGYKKDGLIKDLWPNIRLIQLSGLFISEYYDDYSGLAVLFRKIYSWITAIIIYSQFIFIVIFMV
TKSNDSDQLAAGVVTTLFFTHSMIKFVYFSTGTKSFYRTLSCWNNTSPHPLFAESHSRFHAKSL
SRMRQLLIIVSIVTIFTTISWTTITFFGESVWKVPDPETFNQTMYVPVPRLMLHSWYPWDSGHG
LGYIVAFVLQFYWVFITLSHSNLMELLFSSFLVHACEQLQHLKEILNPLIELSATLDSSVHNPA
EIFRANSAKNQSINGIDHDYNGSYVNEITEYGTKGENEPNRKGPNNLTSNQEVLVRSAIKYWVE
RHKHVVKYVSLITECYGSALLFHMLVSTVILTILAYQATKINGVNVFAFSTIGYLMYSFAQIFM
FCIHGNELIEESSSVMEAAYGCHWYDGSEEAKTFVQIVCQQCQKPLIVSGAKFFNVSLDLFASV
LGAVVTYFMVLVQLK
SEQ ID NO: 13 (OR2 from Acyrthosiphon pisum)
MDVMQKPERFILTPFQKFCIRWSVFFDSSSDRLSRIETVLRTIQFSTIMITSGMTMTSVLIADN
KKALESFTYFVICVFMLAIITFAIRTKRENRAMLLMVVDEFPGYNRPMPDVLKRKMAAIRTSYG
DFTMKVIVSYLTLVLFEIPATAMVPLAAASLTDVKLGSQSTQMVVLWFPADTSQVGMYAVSYVI
QFLIVVTVKFIITGIMCSFSFFVSQMISEFQILSAYVEHAVEIVEYDQSADKTTEQKLLDHVKN
CVMLHDRLIYFKDQLNESYGYIILLELMFSTLYFCLSAFNMIFVGNRFVMVKGLLTLSNYLAEL
FIFCMYGSMVEDAHMGLLRASYSVAWYAQPVRFRQSLTMVMSRTQTPLQLTVGKVFIANLPLFL
SVLKVSYSGVNALRAANAK
SEQ ID NO: 14 (OR4 from Acyrthosiphon pisum)
MTSIGKKNQRKFYQTLMTLAFFLDTSQYRYISRFVKQFYIFDWMVLVSVAAAFTILEGNYRMPF
VMELIQYMIVGFYFTSIFVVFIIKKEAIMSNYNCIQTKFIQWSNKRALHSNAAYKRNIKTVKSL
SIPLAILSLSIALGPLISTINDIGKLPLDNRAHFVLFWPTIVDTNKLSMYGIIYTLQVIFTIIL
YISVLSFNLGYMVFLNELITQFEMLLNGINDAFKYKMDKQFQTLFIDCIRHHQIIIKFLDDLKS
YFKWMILIEIIVVQVILAILIYNLTKVNASLGYKVKIAGSILFNLLPICFHCHVGEVVLSLHTR
LSNHIYNMPWYDMPNKNKQLIVIMLQRTQRDLTLSSALFSSERASRSLISKVIKQVYTILNVLL
KT
SEQ ID NO: 15 (OR5 from Acyrthosiphon pisum)
MQRIDTINMFLQMTGCTDSKAMLYLTYFEFLITFYYLIATYASIVHFEQSVTIQLFALLCMLIE
CVILLNITFRLYHKNHIREMHQYSRRLGIPDSYRSVINVITKYHLIASNIFVVFPVTYAIFCDS
VRVGDPFTFPFLDVLPMHTDNLAIYACKYLVYAISVYIAHVELCFINTTFIYYVGVLKHRLETI
VQTIGEAFADNDEQKFKYAIIQHQKLLSYFNTMKIVFSKPILLSMSFNAIYFGLTTSFVIQAIR
GYINQAILSICIASSAAAVINITIYTFYGSELMDLHDKILHVLFDNAFFYVSKSFKSSILIMMT
RVTIPLKFTVGYIFTINLNLLLKILKMSYTVLNVLLSSETIKPHKLS
SEQ ID NO: 16 (OR10 from Acyrthosiphon pisum)
MAHIVDIFFQNMGCSHDHGYGMVFFNCCELAITLFFTVSTYPTIADPTQNLSIRLYGVLCLLIE
AHIFAFIAVRIYHQSQHRDMYQHLHGVEIPENYRRKIATVIKHHFIISNVFVAVSVLYTISLDW
VRIGDPFTFPFIDVLPIKTTNVTVYVCKYIVYALPVYFAHLETCFLNVTFMFSVGIVKRHFQIL
NDQVEEAIVNEDEQKLKIAIKHHQQVLKYFEDMKTVYEKPILMTIEFCGLYVGLTSCFVIQVIQ
GFIHQIILGLCIVSSIACLMTIIIYCIYASNMYALHNGILNALFEHRSCYSRNKSFKRIILIMM
TRATIPLEIKAGSVFTINLNLLVKILKFAYTVFNVLLSSINRQFKETAI
SEQ ID NO: 17 (OR17 from Acyrthosiphon pisum)
MTTTPRVTELTAPASEDLTIVDNRLFKAICLHQILDPTKGGNRYYRLAFMVVMWVSLSVQIIQL
VGLYFAVNDLQRFAFTTTVIFNALLCLSKGYVLVVNADRLRASLEVARYEFTSCGARNQRLVRR
SRAVLSTILRTFAVLSWVTCFIWALTPLFAMDEYLQVTNADGTVSRYRVTIYNVWLPVPATVYN
ETTVWSLVYAVEVIACFVNVFSWLLFDSYVVTMCFTFNAQFRTVSASTTIGHHSDSFRSPPPHA
PEGTSDDNNTFNCYDELINRIKDNQSIIKIYDDFFEILQPAILFQIIGGSYSVITLIFLTSLTY
LMGFSIISIPVLKVFFGFLSVTFELFLYCYVFNHIETEKCNMNFGLYSSNWTAMDLKFKKTLLF
AMNTNSSHRRVMKVTPMSIINLEMFANVMNMSYSIVSVLLNSRVQK
SEQ ID NO: 18 (OR20 from Acyrthosiphon pisum)
MRSSSATVVDVMLFKAIGLYQLLCPADRGGYSVRSRRALMTALGLSFALHSFQVPYLYYALNDL
QRFAYMAAVIIYGMMCSFKGYVLVTNADRLWLVLNAADYGYTGCGHRDPSRLRRCRATLSALLR
TFVALSYGTLIVWIVLPFFVDEYTGITNSDGTVTRYRTTIHNMQYPIPLAVYNSRPVWALIYVT
ELYVCIVNVFIWSLFDCYLVTMCFVLNAQFHTMSAGYGTLGIRRTGSSPPDTTFAGVRRIKFDE
IESNHYSDLISHIQDNQNLIKMFDVFFEVVRPVVLVQIANGSYSVISLIFLTALMYLMGVPVLS
AAFLKFICGLISLTIELFIFCYGFNHIETAKSVLNFGIYSSNWTEMDLTFKKTMLLTMKMNSSH
KRAMKVSPNSAVGLEMFARVMNMSYSTVSVLLNSRS
SEQ ID NO: 19 (OR22c from Acyrthosiphon pisum)
FKHQGLVADLLPNIRVMQGVGHFMFNYYSEGKKFPHRIYCIVTLLLLLLQYGMMAVNLMMESDD
VDDLTANTITMLFFLHPIVKMIYFPVRSKIFYKTLAIWNNPNSHPLFAESNARFHALAITKMRR
LLFCVAGATIFSVISWTGITFIEDSPIPRLMIRTFYPFNAMSGAGHVFALIYQFYYLVISMAVS
NSLDVLFCSWLLFACEQLQHLKAIMKPLMELSATGLTKKQEMLVRSAIKYWVERHKHVVRLVTA
VGDAYGVALLLHMLTTTITLTLLAYQATKVNGVNVYAATVIGYLLYTLGQVFLFCIFGNRLIEE
SSSVMEAAYSCHWYDGSEEAKTFVQIVCQQCQKAMSISGAKFFTVSLDLFASVLGAVVTYFMVL
VQLK
SEQ ID NO: 20 (OR23 from Acyrthosiphon pisum)
MNLNDEQNYIVNLKLMKITGFYHLISSRAPKYFGFNVYKVTAAIEVMTGIFSIIMLFLSSYYYL
DNTNELMSHFMLVVAIFFSTLKIFWVSRNSETIWNNMDMTCINFLSYTGHKKEILKKARAKSIS
TTILFVILWSSVTVAWSISPFFVKDVYLNIKFKDETRRFRYNSLNYVYPISEEFYNEHFLYFYV
VEMLSVVFWGHGTVAYDTFVISICITIAFQLKTIAVSYISLNDKKGDIKNLKDNDLEAMFNLKL
LIQDQQNMFKKIKEIYKIFEPVTFVQLAAQSMLIILQAYMIFINHYNGFSLLSVPIIKLIVTVA
PNIIHLFITCYLYTNINHQQDSMNFALYSSDWTAMSINYKKMLLFTMRMNDAEKLKLKISLRKI
VNLEMFASVMHLTYSIISVLAKSYGNTNTK
SEQ ID NO: 21 (OR25 from Acyrthosiphon pisum)
MATGIKTVSKNEDNFMINMRLMKKTGFYQLLDSRSLKVFGHNVFKCMSVVQMSILSSVAFIFVA
NIYYFSDDINTVMMYSMLITSDVLSILKLYYILQNSDTIWNCIQMTSIDDLSYKYHDRRILEEG
RSKSTSYSILIMFMWLNLIVSWSLGPLFVTNYFLIVEQNDEIYRYRFNIMNFAFPATDRFYNDN
FMIYYGIEFITLVLWCHCTMNFDVLLLSMNITFKYQLKTISNSFSAFNFTRYNDFKNNRTKNVK
HHKESESMFDFKSLIYDQQRVIENMKNIYRVFRPVVLTQLASESLIIMLLSCIIMLNYFNGISL
LSALNLRIFAAISTFLFHIYVICYLFDDVNEQKDSMNLALYSSDWTTSDLQHKILLLHAMRMNN
AENLRLQVTRNKIVNFQMFTYVRMIFFSFYYSGYCGHYFY
SEQ ID NO: 22 (OR31 from Acyrthosiphon pisum)
MNPTFKHFFKGDCTNITKPSPMETCIDHTCTINLNILKQCGFYQIFDPNSKKIFGWNVYRISFI
ALTVITQCLIGFGNCGFLFELEDTTDNIDLFLIIFSNSYFCLTEWKVVILIINRKKFLELLDVT
DLIFLKSKQCRKNIKILCKHRIRALQLTNLYFKFCIFVIIEWIIFPIMINSFIAHKTENRRLEN
VVNRRYPVDVNTYNKYYILFYVFEIIIGVKTVYLVLMVDILLLSIGWAIVIQYEVLAEAFKNIG
YNENLQKDHDHDVDDYKYFKSILFDQQQLDSKVKLYFPIVKPIVLMHVAINSVLFIMLSNSFLM
VFLSTESFTYKIVNLFKIGTGILYICLQLFLYCHLFDNINLKRKSVNLGIYSCNWTKMDLKFKK
LLLLTMQINDANYITIKASTKTIVNLPIFANVLMTSYNIVSVMVKTMSKYRKT
SEQ ID NO: 23 (OR37 from Acyrthosiphon pisum)
MKWLQDHEVAINLALFKRYQFYQIFNPNGSKLLNYDTYKLTNVMFIVAVTTYNIFSAMCFFTDT
IDTIDSVDLLLMIFIYSIIIISLLKISVLLFNADQIWELFDLTRFDFLTSRQCRKNVGILCKYR
DRSITITNLYQNYSTMVFIIWMITPLVLNTFVVVGGPNQRYHNIFNMQYPVSANIYNQYYYLFY
LMEIAMGIFVLNYSMIVDNFLISLCWVIIAQYEVITTAFEKIGNDCELTTLQNEKNNNSFEAYE
DLKSILMDQNKLYIKLKSFYRVVWIIVIFLIIIDSVLLIILTYSFVMICSSAESFSIFNILKIS
TAFFVFVIQLYLYCYLFDVLNDKKESVNFGLYCCDWTKMDLRFKKLLLLATKFNNANTLKIKST
PNKIVNLQLFSSVMTTAFNIVTVMLKTMNGKN
SEQ ID NO: 24 (OR38 from Acyrthosiphon pisum)
MGIDNMSSLKSNEVAINLKLFKVFRFYHIFDPNSGKLCKFNVYHLAWYIINCVIGCILIYGLLG
YFTEMEDVIDSIFHIQIMFCYLLYSLSLLKIITFLYKANNIWDLLRVTRINFLTSTQCQAHIGI
LHKHRNKSIKITNLISGFAIVTTLEWILFPLVLRLLSKTDASHSNKRFENIFNFRFPVTVCEYN
NYYFIFYIMESFIAIFMLYAYVVTDVFFISVCYVIIAQYEIIKRAYEIVNCEQTSENNNENKNH
NNIIVNDCCDDLISIVMDQQNHYAKLRLFYSTYKLIIVSTVVINSGSIIILTYASVVIFTSPET
IPILSIVKLISAFTYMFFVLFFLCYLMECINNKIESVQLGMYSCNWTAMNIKSKKLLLFSMRMH
NANKLMIKTTPNNIINLQLFNSVMMTSYNIVSAMVNTRSK
SEQ ID NO: 25 (OR39 from Acyrthosiphon pisum)
MFSCDFINRTVNMNSENLFNGGSVAFNLSTYKQLGYYQLLDPKGPHIYGYHLYRTILKIFLLIV
QFITIFGVMGFFIEMEDTDPGKSNSFELIIILTNCSLSSLKIYTLISNSKIIWDLFDLTRIDFL
RCSRHSKLITKNFVKRCKKSTTITKWIARSFLVGLILWLMGPFIANEEHTEPNTVHRHKNIINI
KFPVTMKTYNNYYFVFYLMEVAVGFCIVYGSVLIDAYLMSFCWIISAQYQSVTKAFATFGYNKQ
GSPKDIYKDFKSIIIDHQNIYLKMKSFYAVVRPITLIHVFAYSCSLIMYAYVIVTIFNSKELFI
IAEIMKIVMTVSNVTMEVFIFCYLFELIDNKKEDVNFGLYSCNWTGMDIKFKQLLLMSMKMNNA
NRFKLKASPDVTINRPFFANVIHTCFKIVSVLIQTQSIDLLN
SEQ ID NO: 26 (OR42 from Acyrthosiphon pisum)
MPNSSEECVMSSSMAKCTGLHYIIDPEGPTVGGHNVFHVTVMVMIGFTVVCLSMCPFGLYYWAN
DVTQCIFLLIYIVNFSFGCFKAFTLVRHSDDICRCLDVTRFDFSSGAIMSDPDSARFFRKCRDA
SSTFTGWFAASSHFVLLVWTLLPFVVVGKGVEINNRDGSTSYYHFNPYNMYFLVSSETYNRLHL
VFHLVEWAFGLCFVLIMVAFDTFMVTLCVAITCQMRGIGNAYSKLGHDRCATASNVCSDGGIES
NKSNNEYLRDLKLIIKDHQAVLGKMNDFYKIVGPVILPQLIVASFTIIFVSFIITRNYFNGMLL
TSTQSLKMCCFPIFFYQVYYTCHAFGNLSHRKNVMNFALYSSDWTQMEIKFKKLLLLAMQMHDA
NKLDMKLTDKLVINLELFTRVINMCYSIFSVLVNSQLKIADKQ
SEQ ID NO: 27 (OR43 from Acyrthosiphon pisum)
MDSKQEKQYIFNMKLARIMGLYQILFPNSTSFFGYNIYHVVTVFFVSFTFAISMLFPIGLLYLR
NDIIAIMYYMGCISNFLLSCFKMVNILYHSKDIWKCIDVTSFNYILYKHYDRNVFKNWQTRSIR
ITYIYIVIALFAFFCWIFSPCIMNKSVIAIRNIDGSYSKYRMNIFNLYLIASNETYNKNFYIFY
VIEIIISICYVYFTIVFDVLMLLVCFAISYQLETISNTIKSLGHEIYTRDNIRSGNSIKLKEKH
GILYNDLITIMTDHQNVLKKLNDFYNIFRSITLTQIFIASSSHVFIWFIAAMSIDEGDNADSIL
SFKLFIVLPLINFQLFMTCSLFGTINEKKDSIIFALYSSNWTNMDLKSKKMILFNLTINNASQL
KMKFTNTKIVNLEMFSHTMRFCYSIFSMLINYNKNKMK
SEQ ID NO: 28 (Forward primer for site-directed mutagenesis
(thrombin cleavage site insertion) of OBP6-His6)
cgcggcagCGCTGGGTACGATAGAACATG
SEQ ID NO: 29 (Reverse primer for site-directed mutagenesis
(thrombin cleavage site insertion) of OBP6-His6)
cggcaccagCGGATCCGGACTCTTGTC

1-24. (canceled)

25. A compound of Formula I, or a salt, a solvate, a tautomer, a stereoisomer or a deuterated analogue thereof: 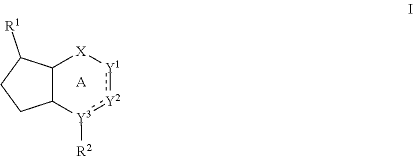
wherein,
X is C═O, C═S, C═S⁺—O—, C(R³)(R⁴), C═C(R⁵)(R⁶) or C═N(R⁷);
in ring A:
(i) Y¹ is C(R⁸)(R⁹), S, S(O), S(O)₂ or N(R¹⁰), Y² is C(R¹¹)(R¹²) and Y³ is C(R¹³); or
(ii) Y¹ is C(R⁸)(R⁹), S, S(O), S(O)₂ or N(R¹⁰), Y² is C(R¹¹) and Y³ is C; or
(iii) Y¹ is C(R⁸) or N, Y² is C(R¹¹) and Y³ is C(R¹³);
R¹ is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R² is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R³ and R⁴ are independently selected from hydrogen, hydroxy, halogen, optionally substituted alkyl, optionally substituted alkoxy, optionally substituted alkanoyl and optionally substituted amino, or R³ and R⁴ together with the carbon atom to which they are attached form a 3-membered or 4-membered optionally substituted carbocyclic or optionally substituted heterocyclic ring;
R⁵ to R¹³ are independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; and [symbol]
represents a single or double bond to maintain correct atom valencies for Y¹, Y² and Y³ in ring A.

26. A compound of Formula IA, or a salt, a solvate, a tautomer, a stereoisomer or a deuterated analogue thereof: 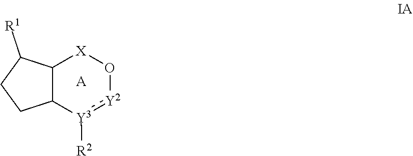
wherein,
X is C═O, C═S, C═S⁺—O—, C(R³)(R⁴), C═C(R⁵)(R⁶) or C═N(R⁷);
in ring A:
(i) Y² is C(R¹¹)(R¹²) and Y³ is C(R¹³); or
(ii) Y² is C(R¹¹) and Y³ is C;
R¹ is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R² is independently selected from hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl;
R³ and R⁴ are independently selected from hydrogen, hydroxy, halogen, optionally substituted alkyl, optionally substituted alkoxy, optionally substituted alkanoyl and amino, or R³ and R⁴ together with the carbon atom to which they are attached form a 3-membered or 4-membered optionally substituted carbocyclic or optionally substituted heterocyclic ring;
R⁵ to R⁷ and R¹¹ to R¹³ are independently selected from hydrogen, hydroxy, halogen, cyano, optionally substituted alkyl, optionally substituted alkenyl, optionally substituted alkynyl, optionally substituted cycloalkyl, optionally substituted cycloalkenyl, optionally substituted heterocycloalkyl, optionally substituted alkoxy, optionally substituted alkanoyl, optionally substituted amino, optionally substituted aryl and optionally substituted heteroaryl; and [symbol]
represents a single or double bond;
wherein the compound of Formula IA is not:

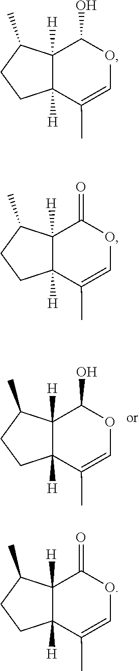

27. A compound according to claim 25, wherein R¹ is C₁-C₆ alkyl or C₁-C₆ haloalkyl; and/or
wherein R² is C₁-C₆ alkyl or C₁-C₆ haloalkyl.

28. A compound according to claim 25 having a structure according to any one of Formulae I-1 to I-7: 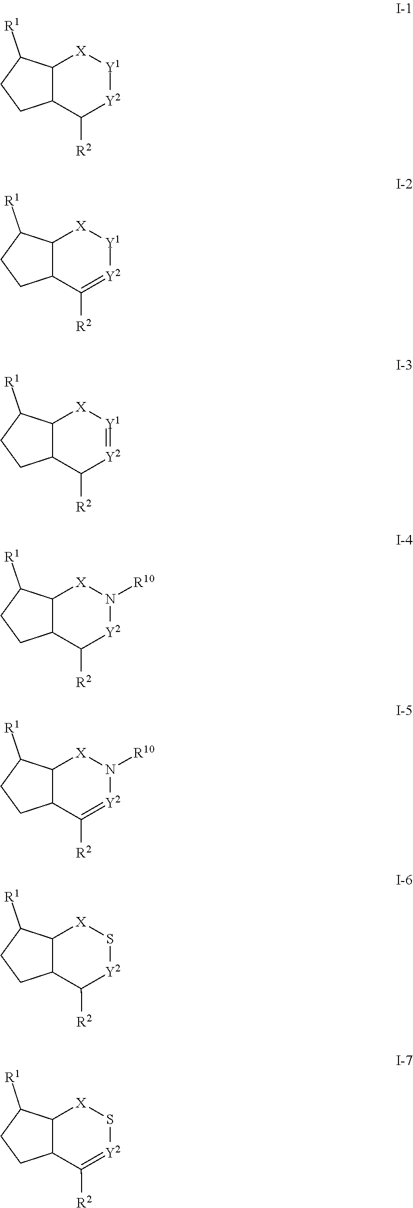
wherein X and R¹ to R¹², Y¹ and Y² are as defined in claim 25.

29. A compound according to claim 25 having a structure according to any one of Formulae II-1 to II-7: 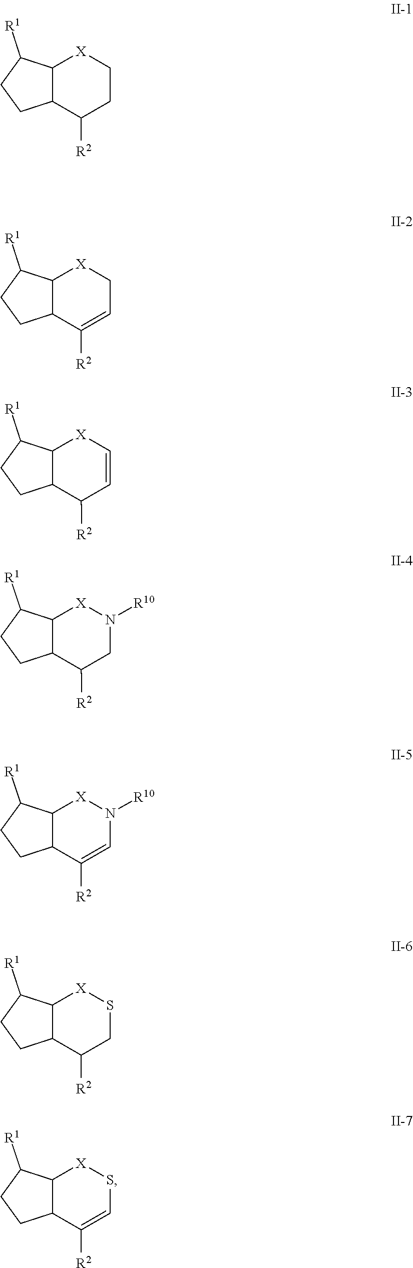
wherein X and R¹ to R⁷ and R¹⁰ are as defined in claim 25.

30. A compound according to claim 25, wherein X is C═O, C═S, CH(OH) or CH(NH₂); and/or
wherein R⁸ is hydrogen or halogen, and/or wherein R⁹ is hydrogen or halogen; and/or
wherein R¹⁰ is hydrogen, hydroxyl or C₁-C₆ alkyl; and/or
wherein R¹¹ is hydrogen or halogen.

31. A compound according to claim 25, having a structure according to any one of Formulae III-1 to III-11 or III-13 to III-20: 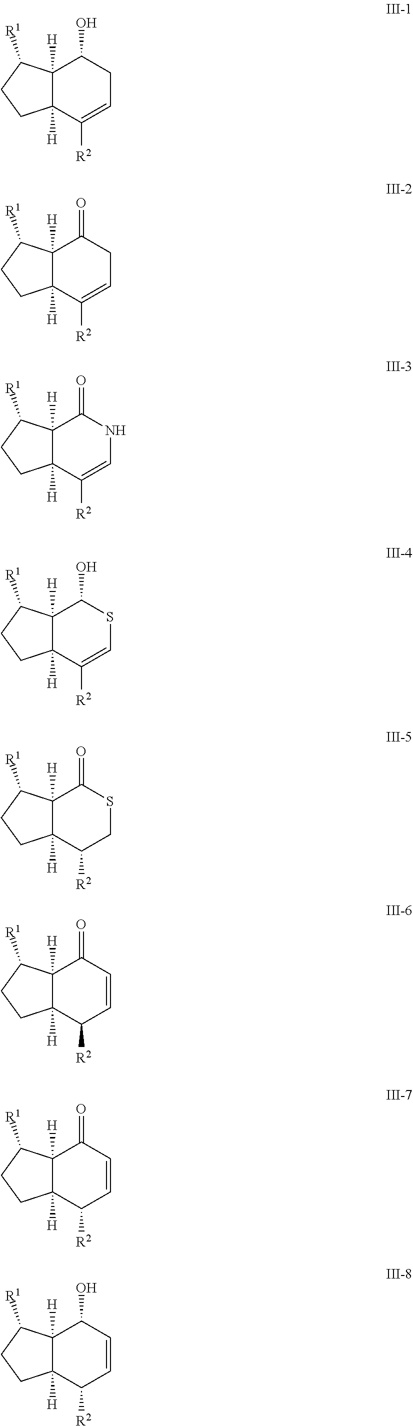 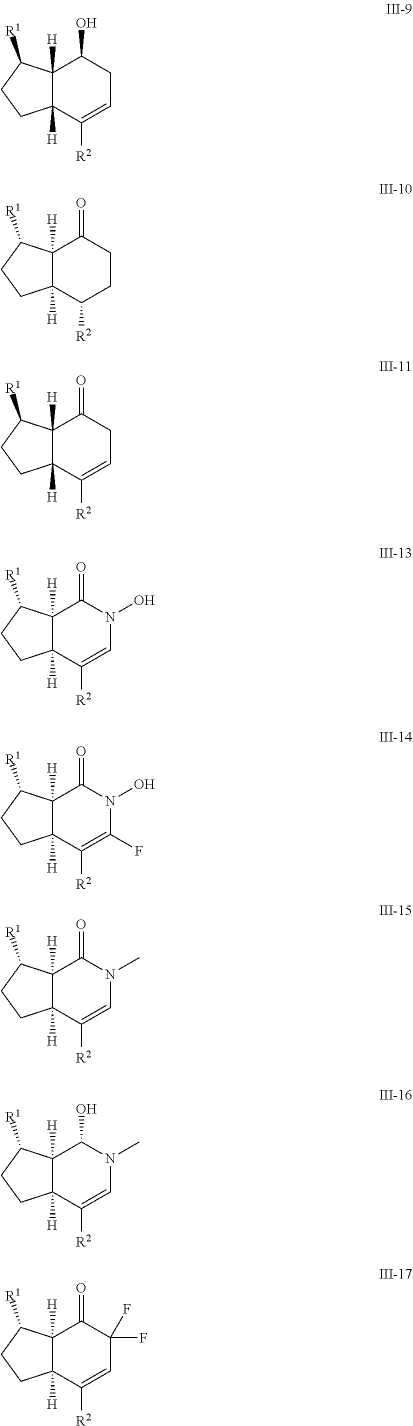 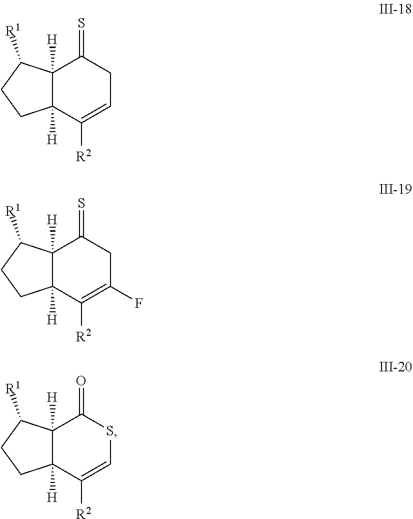
wherein R¹ and R² are as defined in claim 25.

32. A compound according to claim 25, wherein the compound is selected from any one of the following:

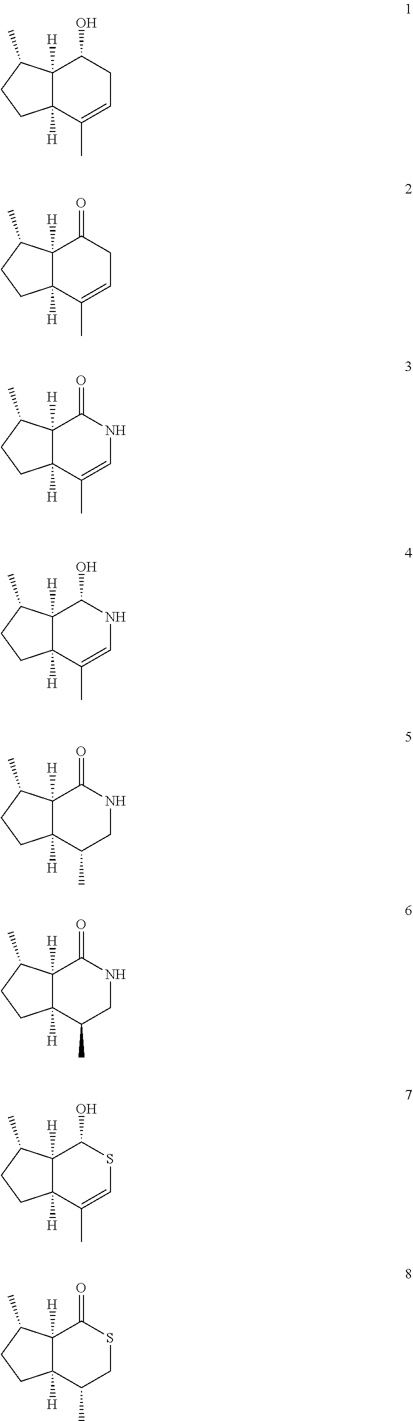

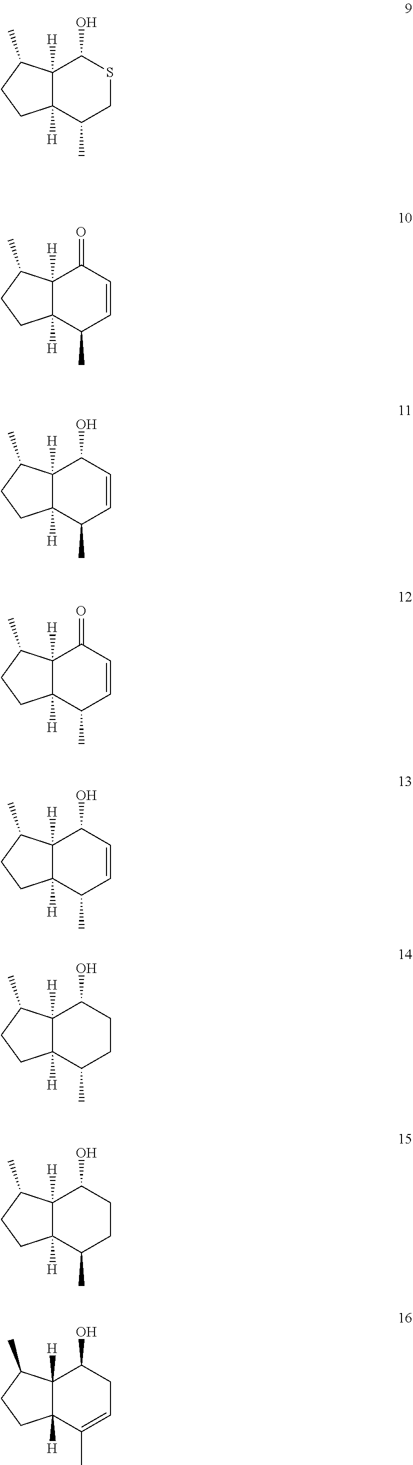

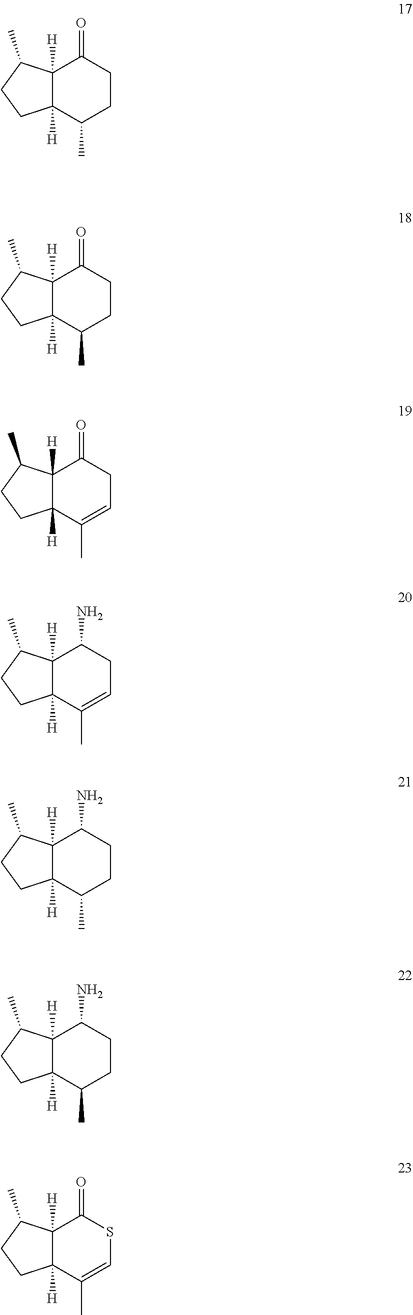

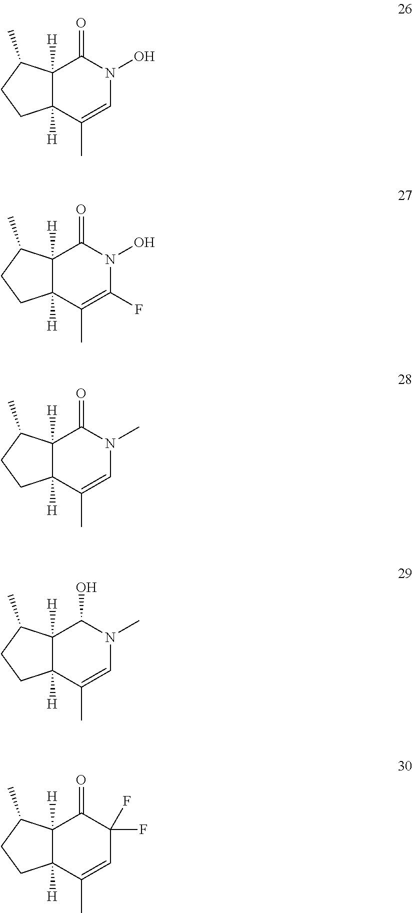

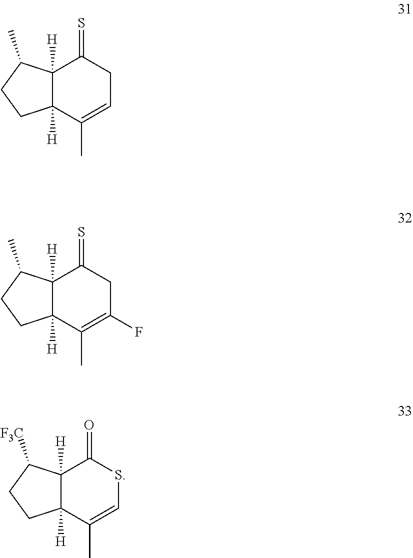

33. A composition comprising a compound according to claim 25 and a carrier.

34. A composition according to claim 33, further comprising at least one additional active ingredient.

35. Use of a compound according to claim 25 to modulate insect behaviour.

36. Use according to claim 35, wherein the insect is selected from aphids, lacewings, houseflies, mosquitoes, cockroaches, mites and ticks.

37. A method of modulating insect behaviour, wherein a compound according to claim 25 is applied to a locus.

38. A method according to claim 37, wherein the compound or composition acts as an insect repellent, insect attractant or insect mating disruptant.

39. A method according to claim 37, wherein the insect is selected from aphids, lacewings, houseflies, mosquitoes, cockroaches, mites or ticks.

40. A biosensor for detecting an analyte in a sample, the biosensor comprising:
a protein having an amino acid sequence as defined in SEQ ID NO: 6, or a fragment or variant thereof; and
a signal generator, wherein the signal generator is configured to output a signal when the analyte is bound to the protein.

41. A biosensor according to claim 40, wherein the biosensor further comprises:
a flow path for moving the sample;
a substrate; and
a protein-containing layer immobilised to the substrate and in contact with the flow path, wherein the protein-containing layer comprises the protein.

42. Use of a biosensor according to claim 40, wherein the biosensor is used to identify olfactory ligands; or wherein the biosensor is used in high-throughput screening;
or wherein the biosensor is used to detect field populations of aphids.

43. A method for detecting an analyte in a sample comprising:
a. providing a biosensor according to claim 40;
b. contacting the biosensor with the sample; and
c. comparing a magnitude of the signal generated by the biosensor when the sample is present with a reference magnitude of the signal generated by the biosensor when the sample is absent.

44. A method according to claim 43, wherein the biosensor is used to identify olfactory ligands; or wherein the biosensor is used in high-throughput screening; or wherein the biosensor is used to detect field populations of aphids.
